(19) United States
(12) Patent Application Publication
(10) Pub. No.: US 2008/0234263 A1
(43) Pub. Date: Sep. 25, 2008

(54) Quinazoline Derivatives

(76) Inventors: Laurent Francois Andre Hennequin, Reims (FR); Christopher Thomas Halsall, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 10/572,303

(22) PCT Filed: Sep. 13, 2004
(86) PCT No.: PCT/GB2004/003911

§ 371 Date: Mar. 17, 2006

(30) Foreign Application Priority Data
Sep. 16, 2003 (GB) 0321621.5
Sep. 25, 2003 (GB) 0322458.1

(51) Int. Cl. A61K 31/5377; C07D 403/08; A61K 31/517; C07D 413/08

(52) U.S. Cl. 514/234.5; 544/293; 514/266.2; 544/119

(57) ABSTRACT

The invention concerns quinazoline derivatives of Formula (I) wherein each of R¹, R³, R²⁰, X¹, X², Z, W, (a) and (q) have any of the meanings defined in the description; processes for their preparation, pharmaceutical compositions containing them and their use in the manufacture of a medicament for use as an antiproliferative agent in the prevention or treatment of turnours which are sensitive to inhibition of erbB receptor tyrosine kinases, particularly EGFR tyrosine kinase.

The invention concerns certain novel quinazoline derivatives, or pharmaceutically-acceptable salts thereof, which possess anti-tumour activity and are accordingly useful in methods of treatment of the human or animal body. The invention also concerns processes for the manufacture of said quinazoline derivatives, to pharmaceutical compositions containing them and to their use in therapeutic methods, for example in the manufacture of medicaments for use in the prevention or treatment of solid tumour disease in a warm-blooded animal such as man.

Many of the current treatment regimes for diseases resulting from the abnormal regulation of cellular proliferation such as psoriasis and cancer, utilise compounds that inhibit DNA synthesis and cellular proliferation. To date, compounds used in such treatments are generally toxic to cells however their enhanced effects on rapidly dividing cells such as tumour cells can be beneficial. Alternative approaches to these cytotoxic anti-tumour agents are currently being developed, for example selective inhibitors of cell signalling pathways. These types of inhibitors are likely to have the potential to display an enhanced selectivity of action against tumour cells and so are likely to reduce the probability of the therapy possessing unwanted side effects.

Eukaryotic cells are continually responding to many diverse extracellular signals that enable communication between cells within an organism. These signals regulate a wide variety of physical responses in the cell including proliferation, differentiation, apoptosis and motility. The extracellular signals take the form of a diverse variety of soluble factors including growth factors as well as paracrine and endocrine factors. By binding to specific transmembrane receptors, these ligands integrate the extracellular signal to the intracellular signalling pathways, therefore transducing the signal across the plasma membrane and allowing the individual cell to respond to its extracellular signals. Many of these signal transduction processes utilise the reversible process of the phosphorylation of proteins that are involved in the promotion of these diverse cellular responses. The phosphorylation status of target proteins is regulated by specific kinases and phosphatases that are responsible for the regulation of about one third of all proteins encoded by the mammalian genome. As phosphorylation is such an important regulatory mechanism in the signal transduction process, it is therefore not surprising that aberrations in these intracellular pathways result in abnormal cell growth and differentiation and so promote cellular transformation (reviewed in Cohen et al, Curr Opin Chem Biol, 1999, 3, 459-465).

It has been widely shown that a number of these tyrosine kinases are mutated to constitutively active forms and/or when over-expressed result in the transformation of a variety of human cells. These mutated and over-expressed forms of the kinase are present in a large proportion of human tumours (reviewed in Kolibaba et al, Biochimica et Biophysica Acta, 1997, 133, F217-F248). As tyrosine kinases play fundamental roles in the proliferation and differentiation of a variety of tissues, much focus has centred on these enzymes in the development of novel anti-cancer therapies. This family of enzymes is divided into two groups—receptor and non-receptor tyrosine kinases e.g. EGF Receptors and the SRC family respectively. From the results of a large number of studies including the Human Genome Project, about 90 tyrosine kinase have been identified in the human genome, of this 58 are of the receptor type and 32 are of the non-receptor type. These can be compartmentalised in to 20 receptor tyrosine kinase and 10 non-receptor tyrosine kinase sub-families (Robinson et al, Oncogene, 2000, 19, 5548-5557).

The receptor tyrosine kinases are of particular importance in the transmission of mitogenic signals that initiate cellular replication. These large glycoproteins, which span the plasma membrane of the cell possess an extracellular binding domain for their specific ligands (such as Epidermal Growth Factor (EGF) for the EGF Receptor). Binding of ligand results in the activation of the receptor's kinase enzymatic activity that is encoded by the intracellular portion of the receptor. This activity phosphorylates key tyrosine amino acids in target proteins, resulting in the transduction of proliferative signals across the plasma membrane of the cell.

It is known that the erbB family of receptor tyrosine kinases, which include EGFR, erbB2, erbB3 and erbB4, are frequently involved in driving the proliferation and survival of tumour cells (reviewed in Olayioye et al., EMBO J., 2000, 19, 3159). One mechanism in which this can be accomplished is by overexpression of the receptor at the protein level, generally as a result of gene amplification. This has been observed in many common human cancers (reviewed in Klapper et al., Adv. Cancer Res., 2000, 77, 25) such as breast cancer (Sainsbury et al., Brit. J. Cancer, 1988, 58, 458; Guerin et al., Oncogene Res., 1988, 3, 21; Slamon et al., Science, 1989, 244, 707; Klijn et al., Breast Cancer Res. Treat., 1994, 29, 73 and reviewed in Salomon et al., Crit. Rev. Oncol. Hematol., 1995, 19, 183), non-small cell lung cancers (NSCLCs) including adenocarcinomas (Cerny et al., Brit. J. Cancer, 1986, 54, 265; Reubi et al., Int. J. Cancer, 1990, 45, 269; Rusch et al., Cancer Research, 1993, 53, 2379; Brabender et al Clin. Cancer Res., 2001, 7, 1850) as well as other cancers of the lung (Hendler et al., Cancer Cells, 1989, 7, 347; Ohsaki et al., Oncol. Rep., 2000, 7, 603), bladder cancer (Neal et al., Lancet, 1985, 366; Chow et al., Clin. Cancer Res., 2001, 7, 1957, Zhau et al., Mol. Carcinog., 3, 254), oesophageal cancer (Mukaida et al., Cancer, 1991, 68, 142), gastrointestinal cancer such as colon, rectal or stomach cancer (Bolen et al., Oncogene Res., 1987, 1, 149; Kapitanovic et al., Gastroenterology, 2000, 112, 1103; Ross et al., Cancer Invest., 2001, 19, 554), cancer of the prostate (Visakorpi et al. Histochem. J., 1992, 24, 481; Kumar et al., 2000, 32, 73; Scher et al., J. Natl. Cancer Inst., 2000, 92, 1866), leukaemia (Konaka et al., Cell, 1984, 37, 1035, Martin-Subero et al., Cancer Genet Cytogenet., 2001, 127, 174), ovarian (Hellstrom et al., Cancer Res., 2001, 61, 2420), head and neck (Shiga et al., Head Neck, 2000, 22, 599) or pancreatic cancer (Ovotny et al., Neoplasma, 2001, 48, 188). As more human tumour tissues are tested for expression of the erbB family of receptor tyrosine kinases it is expected that their widespread prevalence and importance will be further enhanced in the future.

As a consequence of the mis-regulation of one or more of these receptors, it is widely believed that many tumours become clinically more aggressive and so correlate with a poorer prognosis for the patient (Brabender et al. Clin. Cancer Res., 2001, 7, 1850; Ross et al., Cancer Investigation, 2001, 19, 554, Yu et al., Bioessays, 2000, 22.7, 673). In addition to these clinical findings, a wealth of pre-clinical information suggests that the erbB family of receptor tyrosine kinases are involved in cellular transformation. This includes the observations that many tumour cell lines overexpress one or more of the erbB receptors and that EGFR or erbB2 when transfected into non-tumour cells have the ability to transform these cells. This tumourigenic potential has been further verified as transgenic mice that overexpress erbB2 spontaneously develop tumours in the mammary gland. In addition to this, a number of pre-clinical studies have demonstrated that anti-proliferative effects can be induced by knocking out one or more erbB activities by small molecule inhibitors, dominant negatives or inhibitory antibodies (reviewed in Mendelsohn et al., Oncogene, 2000, 19, 6550). Thus it has been recognised that inhibitors of these receptor tyrosine kinases should be of value as a selective inhibitor of the proliferation of mammalian cancer cells (Yaish et al. Science, 1988, 242, 933, Kolibaba et al, Biochimica et Biophysica Acta, 1997, 133, F217-F248; Al-Obeidi et al, 2000, Oncogene, 19, 5690-5701; Mendelsohn et al, 2000, Oncogene, 19, 6550-6565). In addition to this pre-clinical data, findings using inhibitory antibodies against EGFR and erbB2 (c-225 and trastuzumab respectively) have proven to be beneficial in the clinic for the treatment of selected solid tumours (reviewed in Mendelsohn et al, 2000, Oncogene, 19, 6550-6565).

Amplification and/or activity of members of the erbB type receptor tyrosine kinases have been detected and so have been implicated to play a role in a number of non-malignant proliferative disorders such as psoriasis (Ben-Bassat, Curr. Pharm. Des., 2000, 6, 933; Elder et al., Science, 1989, 243, 811), benign prostatic hyperplasia (BPH) (Kumar et al, Int. Urol. Nephrol., 2000, 32, 73), atherosclerosis and restenosis (Bokemeyer et al., Kidney Int., 2000, 58, 549). It is therefore expected that inhibitors of erbB type receptor tyrosine kinases will be useful in the treatment of these and other non-malignant disorders of excessive cellular proliferation.

European patent application EP 566 226 discloses certain 4-anilinoquinazolines that are receptor tyrosine kinase inhibitors.

International patent application publication numbers WO 96/33977, WO 96/33978, WO 96/33979, WO 96/33980, WO 96/33981, WO 97/30034 and WO 97/38994 disclose that certain quinazoline derivatives which bear an anilino substituent at the 4-position and a substituent at the 6- and/or 7-position possess receptor tyrosine kinase inhibitory activity.

European patent application EP 837 063 discloses aryl substituted 4-aminoquinazoline derivatives carrying a moiety containing an aryl or heteroaryl group at the 6- or 7-position on the quinazoline ring. The compounds are stated to be useful for treating hyperproliferative disorders.

International patent application publication numbers WO 97/30035 and WO 98/13354 disclose certain 4-anilinoquinazolines substituted at the 7-position are vascular endothelial growth factor receptor tyrosine kinase inhibitors.

International patent application publication numbers WO 00/55141, WO 00/51991 and WO 02/18372 disclose 6,7-substituted 4-anilinoquinazoline compounds characterised in that the substituents at the 6- and/or 7-position carry an ester linked moiety (RO—CO) or a lactone ring.

International patent application publication number WO 00/56720 discloses 6,7-dialkoxy-4-anilinoquinazoline compounds for the treatment of cancer or allergic reactions.

International patent application publication number WO 02/41882 discloses 4-anilinoquinazoline compounds substituted at the 6- and/or 7-position by a substituted pyrrolidinyl-alkoxy or piperidinyl-alkoxy group.

International patent application publication numbers WO 98/02434, WO 99/35132, WO 00/44728 and WO 01/98277 disclose 4-anilinoquinazolines wherein the anilino group is substituted with an aryl or heteroaryl containing moiety.

We have now surprisingly found that other 4-anilinoquinazoline derivatives possess potent anti-tumour activity. Without wishing to imply that the compounds disclosed in the present invention possess pharmacological activity only by virtue of an effect on a single biological process, it is believed that the compounds provide an anti-tumour effect by way of inhibition of one or more of the erbB family of receptor tyrosine kinases that are involved in the signal transduction steps which lead to the proliferation of tumour cells. In particular, it is believed that the compounds of the present invention provide an anti-tumour effect by way of inhibition of EGFR and/or erbB2 receptor tyrosine kinases.

Generally the compounds of the present invention possess potent inhibitory activity against the erbB receptor tyrosine kinase family, for example by inhibition of EGFR and/or erbB2 and/or erbB4 receptor tyrosine kinases, whilst possessing less potent inhibitory activity against other kinases. Furthermore, certain compounds of the present invention possess substantially better potency against the EGFR over that of the erbB2 tyrosine kinase. The invention also includes compounds that are active against all or a combination of EGFR, erbB2 and erbB4 receptor tyrosine kinases, thus potentially providing treatments for conditions mediated by one or more of these receptor tyrosine kinases.

Generally the compounds of the present invention exhibit favourable physical properties such as a high solubility whilst retaining high antiproliferative activity. Many of the compounds of the invention posses favourable DMPK properties, for example high bioavailability and/or high free-plasma levels and/or advantageous half life and such properties are expected to provide improved in-vivo efficacy and may reduce inter-patient variability in exposure to the compound compared to other EGFR tyrosine kinase inhibitors such as gefitinib. Furthermore, many of the compounds according to the present invention are inactive or only weakly active in a hERG assay.

According to a first aspect of the invention there is provided a quinazoline derivative of the Formula I:

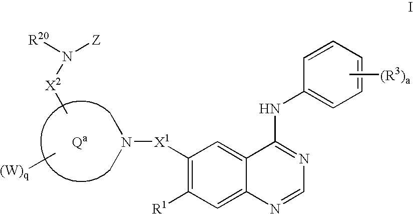

wherein:

R¹ is selected from hydrogen, hydroxy, (1-6C)alkoxy, (2-6C)alkenyloxy, (2-6C)alkynyloxy, or a group of the formula:

Q¹-X³—

wherein X³ is O or S, and Q¹ is (3-7C)cycloalkyl, (3-7C)cycloalkyl-(1-6C)alkyl, (3-7C)cycloalkenyl, (3-7C)cycloalkenyl-(1-6C)alkyl, heterocyclyl or heterocyclyl-(1-6C)alkyl,

and wherein adjacent carbon atoms in any (2-6C)alkylene chain within a R¹ substituent are optionally separated by the insertion into the chain of a group selected from O, S, SO, SO₂, N(R⁴), CO, CH(OR⁴), CON(R⁴), N(R⁴)CO, SO₂N(R⁴), N(R⁴)SO₂, CH═CH and C≡C wherein R⁴ is hydrogen or (1-6C)alkyl,

and wherein any CH₂═CH— or HC≡C— group within a R¹ substituent optionally bears at the terminal CH₂═ or HC≡ position a substituent selected from halogeno, carboxy, carbamoyl, (1-6C)alkoxycarbonyl, N-(1-6C)alkylcarbamoyl, N,N-di-[(1-6C)alkyl]carbamoyl, amino-(1-6C)alkyl, (1-6C)alkylamino-(1-6C)alkyl and di-[(1-6C)alkyl]amino-(1-6C)alkyl or from a group of the formula:

Q²-X⁴—

wherein X⁴ is a direct bond or is selected from CO and N(R⁵)CO, wherein R⁵ is hydrogen or (1-6C)alkyl, and Q² is heterocyclyl or heterocyclyl-(1-6C)alkyl,

and wherein any alkyl or alkylene group within a R¹ substituent optionally bears one or more halogeno, (1-6C)alkyl, hydroxy, cyano, amino, carboxy, carbamoyl, sulfamoyl, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino, or from a group of the formula:

—X⁵-Q³

wherein X⁵ is a direct bond or is selected from O, S, SO, SO₂, N(R⁶), CO, CH(OR⁶), CON(R⁶), N(R⁶)CO, SO₂N(R⁶), N(R⁶)SO₂, C(R⁶)₂O, C(R⁶)₂S and C(R⁶)₂N(R⁶), wherein R⁶ is hydrogen or (1-6C)alkyl, and Q³ is (3-7C)cycloalkyl, (3-7C)cycloalkyl-(1-6C)alkyl, (3-7C)cycloalkenyl, (3-7C)cycloalkenyl-(1-6C)alkyl, heterocyclyl or heterocyclyl-(1-6C)alkyl,

and wherein any heterocyclyl group within a substituent on R¹ optionally bears 1, 2 or 3 substituents, which may be the same or different, selected from halogeno, trifluoromethyl, cyano, nitro, hydroxy, amino, carboxy, carbamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino, and N-(1-6C)alkyl-(1-6C)alkanesulfonylamino, or from a group of the formula:

—X⁶—R⁷

wherein X⁶ is a direct bond or is selected from O, N(R⁸) and C(O), wherein R⁸ is hydrogen or (1-6C)alkyl, and R⁷ is halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, (2-6C)alkanoyl-(1-6C)alkyl or (1-6C)alkoxycarbonyl-(1-6C)alkyl,

and wherein any heterocyclyl group within a substituent on R¹ optionally bears 1 or 2 oxo or thioxo substituents;

X¹ is (C(R⁹)₂)ₙ, wherein each R⁹, which may be the same or different, is selected from hydrogen, hydroxy, (1-4C)alkoxy, (1-4C)alkyl, halo(1-4C)alkyl, hydroxy (1-4C)alkyl, (1-4C)alkoxy(1-4C)alkyl, (3-7C)cycloalkyl and (3-7C)cycloalkyl-(1-4C)alkyl, or two groups R⁹ together with the carbon atom(s) to which they are attached form a (3-7C)cycloalkyl ring, and n is 1 or 2, provided that when a group R⁹ is hydroxy or (1-4C)alkoxy, n is 2, and the carbon atom to which the hydroxy or (1-4C)alkoxy group is attached is not also attached to another oxygen or a nitrogen atom;

Q^{a} is a non-aromatic saturated or partially unsaturated heterocyclyl group containing 1 nitrogen heteroatom and optionally 1, 2 or 3 additional heteroatoms selected from O, S and N, and which group is linked to X¹ in Formula I by the nitrogen heteroatom in Q^{a};

q is 0, 1, 2, 3 or 4;

each W, which may be the same or different, is selected from halogeno, trifluoromethyl, cyano, nitro, hydroxy, oxo, amino, carboxy, carbamoyl, sulfamoyl, formyl, mercapto, (1-6C)alkyl, (1-6C)alkoxy, (2-6C)alkenyl, (2-6C)alkynyl, (2-6C)alkenyloxy, (2-6C)alkynyloxy, (1-6C)alkoxycarbonyl, (1-6C)alkylthio, (1-6C)alkylsulfinyl, (1-6C)alkylsulfonyl, N-(1-6C)alkylamino, N,N-di-[(1-6C)alkyl]amino, N-(1-6C)alkylcarbamoyl, N,N-di-[(1-6C)alkyl]carbamoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino, and N-(1-6C)alkyl-(1-6C)alkanesulfonylamino, or from a group of the formula:

—X⁷—R¹⁰

wherein X⁷ is a direct bond or is selected from O, CO and N(R¹¹), wherein R¹¹ is hydrogen or (1-6C)alkyl, and R¹⁰ is selected from (1-6C)alkyl optionally substituted by one or more groups selected from halogeno, hydroxy, (1-6C)alkoxy, cyano, amino, N-(1-6C)alkylamino, N,N-di-[(1-6C)alkyl]amino, (2-6C)alkanoylamino, carbamoyl, N-(1-6C)alkylcarbamoyl, N,N-di-[(1-6C)alkyl]carbamoyl, (2-6C)alkanoyl and (2-6C)alkanoyloxy,

or two W groups form a (1-4C)alkylene bridge, which (1-4C)alkylene bridge optionally bears 1, 2 or 3 substituents, which may be the same or different, selected from halogeno, hydroxy, oxo, (1-6C)alkyl, (1-6C)alkoxy, amino, N-(1-6C)alkylamino and N,N-di-[(1-6C)alkyl]amino;

X² is selected from CH₂C(O), CH₂SO₂, C(O) and SO₂;

Z is selected from hydrogen, (1-6C)alkyl, (2-6C)alkenyl, (2-6C)alkynyl, (3-7C)cycloalkyl, (3-7C)cycloalkyl-(1-4C)alkyl, heterocyclyl, heterocyclyl-(1-4C)alkyl, aryl and aryl-(1-4C)alkyl,

and wherein adjacent carbon atoms in any (2-6C)alkylene chain within a Z substituent are optionally separated by the insertion into the chain of a group selected from O, S, SO, SO₂, N(R¹²) and CO, wherein R¹² is selected from hydrogen and (1-6C)alkyl,

and wherein any CH₂═CH— or HC≡C— group within a Z substituent optionally bears at the terminal CH₂═ or HC≡ position a substituent selected from halogeno, carboxy, carbamoyl,

and wherein any alkyl, alkylene or (3-7C)cycloalkyl group within a Z substituent, optionally bears on one or more halogeno or (1-6C)alkyl substituents or a substituent selected from hydroxy, cyano, amino, carboxy, carbamoyl, sulfamoyl, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino, N-(1-6C)alkyl-(1-6C)alkanesulfonylamino, (3-7C)cycloalkyl and heterocyclyl,

and wherein any aryl or heterocyclyl group within a Z substituent optionally bears one or more substituents selected from halogeno, cyano, nitro, hydroxy, amino, carboxy, carbamoyl, sulfamoyl, trifluoromethyl, (1-4C)alkyl, (2-4C)alkenyl, (2-4C)alkynyl, (1-3C)alkoxy, (1-4C)alkylthio, (1-4C)alkylsulfinyl, (1-4C)alkylsulfonyl, (2-6C)alkaoyl, (1-4C)alkylamino, di-[(1-4C)alkyl]amino, (1-4C)alkoxycarbonyl, N-(1-6C)alkylcarbamoyl and N,N-di-[(1-6C)alkyl]carbamoyl,

and wherein any heterocyclyl group within a Z substituent optionally bears 1 or 2 oxo or thioxo substituents, provided that any of said oxo substituents are not on a carbon atom adjacent to a ring oxygen in the heterocyclyl group;

R²⁰ is hydrogen, (1-6C)alkyl, hydroxy-(2-6C)alkyl or (1-6C)alkoxy(2-6C)alkyl;

a is 1, 2, 3, 4 or 5;

each R³, which may be the same or different, is selected from halogeno, cyano, nitro, hydroxy, amino, carboxy, carbamoyl, sulfamoyl, trifluoromethyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, N-(1-6C)alkylsulfamoyl, and N,N-di-[(1-6C)alkyl]sulfamoyl;

or a pharmaceutically acceptable salt thereof.

According to a further aspect of the invention there is provided a quinazoline derivative of the Formula I of the Formula IA:

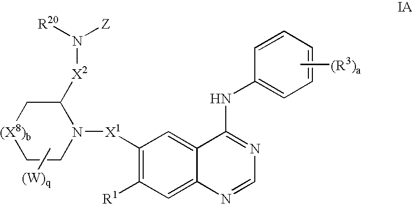

wherein:

R¹ is selected from hydrogen, hydroxy, (1-6C)alkoxy, (2-6C)alkenyloxy, (2-6C)alkynyloxy, or a group of the formula:

Q¹-X³—

wherein X³ is O or S, and Q¹ is (3-7C)cycloalkyl, (3-7C)cycloalkyl-(1-6C)alkyl, (3-7C)cycloalkenyl, (3-7C)cycloalkenyl-(1-6C)alkyl, heterocyclyl or heterocyclyl-(1-6C)alkyl,

and wherein adjacent carbon atoms in any (2-6C)alkylene chain within a R¹ substituent are optionally separated by the insertion into the chain of a group selected from O, S, SO, SO₂, N(R⁴), CO, CH(OR⁴), CON(R⁴), N(R⁴)CO, SO₂N(R⁴), N(R⁴)SO₂, CH═CH and C≡C wherein R⁴ is hydrogen or (1-6C)alkyl,

and wherein any CH₂═CH— or HC≡C— group within a R¹ substituent optionally bears at the terminal CH₂═ or HC≡ position a substituent selected from halogeno, carboxy, carbamoyl, (1-6C)alkoxycarbonyl, N-(1-6C)alkylcarbamoyl, N,N-di-[(1-6C)alkyl]carbamoyl, amino-(1-6C)alkyl, (1-6C)alkylamino-(1-6C)alkyl and di-[(1-6C)alkyl]amino-(1-6C)alkyl or from a group of the formula:

Q²-X⁴—

wherein X⁴ is a direct bond or is selected from CO and N(R⁵)CO, wherein R⁵ is hydrogen or (1-6C)alkyl, and Q² is heterocyclyl or heterocyclyl-(1-6C)alkyl,

and wherein any alkyl or alkylene group within a R¹ substituent optionally bears one or more halogeno, (1-6C)alkyl, hydroxy, cyano, amino, carboxy, carbamoyl, sulfamoyl, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino, or from a group of the formula:

—X⁵-Q³

wherein X⁵ is a direct bond or is selected from O, S, SO, SO₂, N(R⁶), CO, CH(OR⁶), CON(R⁶), N(R⁶)CO, SO₂N(R⁶), N(R⁶)SO₂, C(R⁶)₂O, C(R⁶)₂S and C(R⁶)₂N(R⁶), wherein R⁶ is hydrogen or (1-6C)alkyl, and Q³ is (3-7C)cycloalkyl, (3-7C)cycloalkyl-(1-6C)alkyl, (3-7C)cycloalkenyl, (3-7C)cycloalkenyl-(1-6C)alkyl, heterocyclyl or heterocyclyl-(1-6C)alkyl,

and wherein any heterocyclyl group within a substituent on R¹ optionally bears 1, 2 or 3 substituents, which may be the same or different, selected from halogeno, trifluoromethyl, cyano, nitro, hydroxy, amino, carboxy, carbamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino, and N-(1-6C)alkyl-(1-6C)alkanesulfonylamino, or from a group of the formula:

—X⁶—R⁷

wherein X⁶ is a direct bond or is selected from O, N(R⁸) and C(O), wherein R⁸ is hydrogen or (1-6C)alkyl, and R⁷ is halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, (2-6C)alkanoyl-(1-6C)alkyl or (1-6C)alkoxycarbonyl-(1-6C)alkyl,

and wherein any heterocyclyl group within a substituent on R¹ optionally bears 1 or 2 oxo or thioxo substituents;

X¹ is (C(R⁹)₂)ₙ, wherein each R⁹, which may be the same or different, is selected from hydrogen, hydroxy, (1-4C)alkyl, halo(1-4C)alkyl, hydroxy (1-4C)alkyl, (1-4C)alkoxy(1-4C)alkyl, and n is 1 or 2, or two groups R⁹ together with the carbon atom(s) to which they are attached form a (3-7C)cycloalkyl ring, provided that when a group R⁹ is hydroxy, n is 2;

each W, which may be the same or different, is selected from halogeno, trifluoromethyl, cyano, nitro, hydroxy, oxo, amino, carboxy, carbamoyl, sulfamoyl, formyl, mercapto, (1-6C)alkyl, (2-6C)alkenyl, (2-6C)alkynyl, (2-6C)alkenyloxy, (2-6C)alkynyloxy, (1-6C)alkoxycarbonyl, (1-6C)alkylthio, (1-6C)alkylsulfinyl, (1-6C)alkylsulfonyl, N-(1-6C)alkylcarbamoyl, N,N-di-[(1-6C)alkyl]carbamoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino, and N-(1-6C)alkyl-(1-6C)alkanesulfonylamino, or from a group of the formula:

—X⁷—R¹⁰

wherein X⁷ is a direct bond or is selected from O, CO and N(R¹¹), wherein R¹¹ is hydrogen or (1-6C)alkyl, and R¹⁰ is (1-6C)alkyl optionally substituted by one or more groups selected from halogeno, hydroxy, (1-6C)alkoxy, cyano, amino, N-(1-6C)alkylamino, N,N-di-[(1-6C)alkyl]amino, (2-6C)alkanoylamino, carbamoyl, N-(1-6C)alkylcarbamoyl, N,N-di-[(1-6C)alkyl]carbamoyl, (2-6C)alkanoyl and (2-6C)alkanoyloxy,

X² is selected from C(O) and SO₂;

Z is selected from hydrogen, (1-6C)alkyl, (2-6C)alkenyl, (2-6C)alkynyl,

and wherein adjacent carbon atoms in any (2-6C)alkylene chain within a Z substituent are optionally separated by the insertion into the chain of a group selected from O, S, SO, SO₂, N(R¹²) and CO, wherein R¹² is selected from hydrogen and (1-6C)alkyl,

and wherein any CH₂═CH— or HC≡C— group within a Z substituent optionally bears at the terminal CH₂═ or HC≡ position a substituent selected from halogeno, carboxy, carbamoyl,

and wherein any alkyl or alkylene group within a Z substituent, optionally bears on one or more halogeno or (1-6C)alkyl substituents or a substituent selected from hydroxy, cyano, amino, carboxy, carbamoyl, sulfamoyl, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino or (3-8)cycloalkyl or heterocyclyl, either of which may be optionally substituted by one or more groups selected from halogeno, cyano, nitro, hydroxy, amino, carboxy, carbamoyl, sulfamoyl, trifluoromethyl, (1-4C)alkyl, (2-4C)alkenyl, (2-4C)alkynyl, (1-3C)alkoxy, (2-4C)alkenyloxy, (2-4C)alkynyloxy, (1-4C)alkylthio, (1-4C)alkylsulfinyl, (1-4C)alkylsulfonyl, (1-4C)alkylamino, di-[(1-4C)alkyl]amino, (1-4C)alkoxycarbonyl;

each R³, which may be the same or different, is selected from halogeno, cyano, nitro, hydroxy, amino, carboxy, carbamoyl, sulfamoyl, trifluoromethyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, N-(1-6C)alkylsulfamoyl, and N,N-di-[(1-6C)alkyl]sulfamoyl

X⁸ is selected from CH₂, O or NR³ where R¹³ is hydrogen, halogeno, trifluoromethyl, carboxy, carbamoyl, sulfamoyl, formyl, mercapto, (1-6C)alkyl, (2-6C)alkenyl, (2-6C)alkynyl, (2-6C)alkenyloxy, (2-6C)alkynyloxy, (1-6C)alkoxycarbonyl, (1-6C)alkylthio, (1-6C)alkylsulfinyl, (1-6C)alkylsulfonyl, N-(1-6C)alkylcarbamoyl, N,N-di-[(1-6C)alkyl]carbamoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino, and N-(1-6C)alkyl-(1-6C)alkanesulfonylamino, or from a group of the formula:

—X⁷—R¹⁰

where X⁷ and R¹⁰ are as defined above;

a is 1, 2, 3, 4 or 5;

b is 0 or 1;

q is 0, 1, 2, 3 or 4; and

R²⁰ is hydrogen, (1-6C)alkyl, or (1-6C)alkoxy(2-6C)alkyl;

or a pharmaceutically acceptable salt thereof.

Preferably in the quinazoline of Formula IA when a group R⁹ is hydroxy, n is 2, and the carbon atom to which the hydroxy or (1-4C)alkoxy group is attached is not also attached to another oxygen or a nitrogen atom.

In this specification the generic term “alkyl” includes both straight-chain and branched-chain alkyl groups such as propyl, isopropyl and tert-butyl, and (3-8C)cycloalkyl groups such as cyclopropyl, cyclobutyl, cyclopentyl, cyclohexyl and cycloheptyl. However references to individual alkyl groups such as “propyl” are specific for the straight-chain version only, references to individual branched-chain alkyl groups such as “isopropyl” are specific for the branched-chain version only and references to individual cycloalkyl groups such as “cyclopentyl” are specific for that 5-membered ring only. An analogous convention applies to other generic terms, for example (1-6C)alkoxy includes methoxy, ethoxy, cyclopropyloxy and cyclopentyloxy, (1-6C)alkylamino includes methylamino, ethylamino, cyclobutylamino and cyclohexylamino, and di-[(1-6Calkyl]amino includes dimethylamino, diethylamino, N-cyclobutyl-N-methylamino and N-cyclohexyl-N-ethylamino.

The term “aryl” refers to aromatic hydrocarbon rings such as phenyl or naphthyl, particularly phenyl. The terms “heterocyclic” or “heterocyclyl” include ring structures that may be mono- or bicyclic and contain from 3 to 15 atoms, at least one of which, and suitably from 1 to 4 of which, is a heteroatom such as oxygen, sulfur or nitrogen. Unless specified otherwise herein, rings within a heterocyclyl group may be aromatic, non-aromatic or partially aromatic in the sense that one ring of a fused ring system may be aromatic and the other non-aromatic. Particular examples of such ring systems include furyl, benzofuranyl, tetrahydrofuryl, chromanyl, thienyl, benzothienyl, pyridyl, piperidinyl, quinolyl, 1,2,3,4-tetrahydroquinolinyl, isoquinolyl, 1,2,3,4-tetrahydroisoquinolinyl, pyrazinyl, piperazinyl, pyrimidinyl, pyridazinyl, quinoxalinyl, quinazolinyl, cinnolinyl, pyrrolyl, pyrrolidinyl, indolyl, indolinyl, imidazolyl, benzimidazolyl, pyrazolyl, indazolyl, oxazolyl, benzoxazolyl, isoxazolyl, thiazolyl, benzothiazolyl, isothiazolyl, morpholinyl, 4H-1,4-benzoxazinyl, 4H-1,4-benzothiazinyl, 1,2,3-triazolyl, 1,2,4-triazolyl, oxadiazolyl, furazanyl, thiadiazolyl, tetrazolyl, dibenzofuranyl, dibenzothienyl oxiranyl, oxetanyl, azetidinyl, tetrahydropyranyl, oxepanyl, oxazepanyl, 1,3-thiazolidinyl, tetrahydro-1,4-thiazinyl, 1,1-dioxotetrahydro-1,4-thiazinyl, homopiperidinyl, homopiperazinyl, dihydropyridinyl, tetrahydropyridinyl, dihydropyrimidinyl, tetrahydropyrimidinyl, tetrahydrothienyl, tetrahydrothiopyranyl or thiomorpholinyl.

Where rings include nitrogen atoms, these may carry a hydrogen atom or a substituent group such as an (1-6C)alkyl group if required to fulfil the bonding requirements of nitrogen, or they may be linked to the rest of the structure by way of the nitrogen atom. A nitrogen atom within a heterocyclyl group may be oxidized to give the corresponding N oxide.

The term “heteroaryl” used herein refers to heterocyclyl groups which are completely aromatic in nature. Particular examples of such ring systems include furyl, benzofuranyl, thienyl, benzothienyl, pyridyl, quinolyl, isoquinolyl, pyrazinyl, pyrimidinyl, pyridazinyl, quinoxalinyl, quinazolinyl, cinnolinyl, pyrrolyl, indolyl, indolinyl, imidazolyl, benzimidazolyl, pyrazolyl, indazolyl, oxazolyl, benzoxazolyl, isoxazolyl, thiazolyl, benzothiazolyl, isothiazolyl, 1,2,3-triazolyl, 1,2,4-triazolyl, oxadiazolyl, furazanyl, thiadiazolyl, tetrazolyl, dibenzofuranyl or dibenzothienyl

Where, for example Z is heteroaryl, or contains a heteroaryl group, said heteroaryl group is suitably a 5 or 6-membered heteroaryl group which contains one or more heteroatoms selected from oxygen, nitrogen or sulfur. Particular 5 or 6 membered heteroaryl groups include those selected from furyl, thienyl, pyridyl, pyrazinyl, pyrimidinyl, pyridazinyl, pyrrolyl, imidazolyl, pyrazolyl, oxazolyl, isoxazolyl, thiazolyl, isothiazolyl, 1,2,3-triazolyl, 1,2,4-triazolyl, oxadiazolyl, furazanyl, thiadiazolyl, tetrazolyl. The heteroaryl group may also be a 9 or 10 membered bicyclic heteroaryl ring system such as quinolinyl, isoquinolinyl, cinnolinyl, quinazolinyl, phthalazinyl, quinoxalinyl, indolyl, isoindolyl, benzofuranyl, benzothienyl, benzimidazolyl, benzothiazolyl or purinyl.

Particular examples of heteroaryl include 5-membered rings such as furyl, thienyl, pyrrolyl, pyrazolyl, imidazolyl, oxazolyl, isoxazolyl, thiazolyl, isothiazolyl, 1,2,3-triazolyl, 1,2,4-triazolyl, oxadiazolyl, furazanyl, thiadiazolyl or tetrazolyl.

Further examples of heteroaryl include 9- or 10-membered bicyclic ring systems such as indolyl, quinolinyl, benzofuranyl, or benzothienyl.

More particular heteroaryl groups are selected from isoxazolyl, furyl, thienyl, pyridyl, pyrazolyl, pyrrolyl, indolyl, quinolinyl, benzofuranyl and benzothienyl.

In particular embodiments of the invention when any of the Q groups defined herein (for example Q¹, Q^{a}, Q² or Q³) in Formula (I) is heterocyclyl, they are a non-aromatic saturated (i.e. with the maximum degree of saturation) or partially saturated (i.e. ring systems retaining some, but not the full, degree of unsaturation) 3 to 10 membered monocyclic ring with up to five heteroatoms selected from oxygen, nitrogen and sulfur (but not containing any O—O, O—S or S—S bonds), and linked via a ring carbon atom, or a ring nitrogen atom (provided the ring is not thereby quaternised). Suitable values for Q¹, Q² or Q³ include for example, oxiranyl, oxetanyl, azetidinyl, tetrahydrofuranyl, tetrahydropyranyl, oxepanyl, oxazepanyl, pyrrolinyl, pyrrolidinyl, morpholinyl, tetrahydro-1,4-thiazinyl, 1,1-dioxotetrahydro-1,4-thiazinyl, piperidinyl, homopiperidinyl, piperazinyl, homopiperazinyl, dihydropyridinyl, tetrahydropyridinyl, dihydropyrimidinyl, tetrahydropyrimidinyl, tetrahydrothienyl, 1,3-thiazolidinyl, tetrahydrothiopyranyl, thiomorpholinyl, more specifically including for example, tetrahydrofuran-3-yl, tetrahydrofuran-2-yl-, tetrahydropyran-4-yl, tetrahydrothien-3-yl, 1,3-thiazolidin-3-yl, tetrahydrothiopyran-4-yl, pyrrolidin-3-yl, pyrrolidin-2-yl, 3-pyrrolin-3yl-, morpholino, 1,1-dioxotetrahydro-4H-1,4-thiazin-4-yl, piperidino, piperidinyl, piperidin-3-yl, piperidin-2-yl, homopiperidin-3-yl, homopiperidin-4-yl, piperazin-1-yl, 1,4-oxazepanyl, or 1,2,3,6-tetrahydropyridin-4-yl. A nitrogen or sulfur atom within a heterocyclyl group may be oxidized to give the corresponding N or S oxide(s), for example 1,1-dioxotetrahydrothienyl, 1-oxotetrahydrothienyl, 1,1-dioxotetrahydrothiopyranyl or 1-oxotetrahydrothiopyranyl. A suitable value for such a group which bears 1 or 2 oxo or thioxo substituents is, for example, 2-oxopyrrolidinyl, 2-oxopiperazinyl, 2-thioxopyrrolidinyl, 2-oxopiperidinyl, 2,5-dioxopyrrolidinyl or 2,6-dioxopiperidinyl.

Particular values for Q¹, Q² and Q³ include, for example, non-aromatic saturated or partially saturated 3 to 7 membered monocyclic heterocyclyl rings with 1 ring nitrogen or sulfur heteroatom and optionally 1 or 2 heteroatoms selected from nitrogen, oxygen and sulfur. Examples of such rings include azetidinyl, oxazepanyl, pyrrolinyl, pyrrolidinyl, morpholinyl, 1,3-thiazolidinyl, tetrahydro-1,4-thiazinyl, piperidinyl, homopiperidinyl, piperazinyl, homopiperazinyl, dihydropyridinyl, tetrahydropyridinyl, dihydropyrimidinyl, tetrahydropyrimidinyl, tetrahydrothienyl, tetrahydrothiopyranyl or thiomorpholinyl.

Particular values for Q¹, Q² or Q³ include, for example, morpholino, or 4, 5 or 6 membered heterocyclyl rings containing 1 nitrogen atom and optionally 1 or 2 heteroatoms selected from nitrogen and sulfur such as azetidinyl, 1,3-thiazolidinyl, piperazinyl, pyrrolidinyl, piperidinyl, particularly azetidin-1-yl, pyrrolidin-1-yl, pyrrolidin-2-yl, piperazin-1-yl or piperidino. More particularly suitable values for any of Q¹, Q² or Q³ include, for example, morpholino, pyrrolidin-1-yl, pyrrolidin-2-yl, piperazin-1-yl, piperidino, piperidin-3-yl, or piperidinyl.

As will be understood, the nitrogen atom attached to X¹ in formula I is a ring nitrogen in the heterocyclyl group Q^{a}. Accordingly ring represented by the group Q^{a} contains 1 nitrogen heteroatom which is linked to X¹ and optionally contains 1, 2 or 3 additional ring heteroatoms selected from O, S and N. A particular value for Q^{a} is a non-aromatic 4, 5, 6 or 7 membered monocyclic heterocyclyl group containing 1 nitrogen heteroatom and optionally 1 or 2 further heteroatoms selected from oxygen, nitrogen and sulfur, which heterocyclyl group may be fully saturated or partially saturated and which is nitrogen linked to the group X¹ in Formula I. More particularly Q^{a} is a non-aromatic nitrogen linked 4, 5 or 6 membered monocyclic heterocyclyl group containing 1 nitrogen heteroatom and optionally 1 further heteroatom selected from oxygen, nitrogen and sulfur, which heterocyclyl group may be partially saturated or preferably fully saturated. Still more particularly Q^{a} is a nitrogen linked monocyclic fully saturated 4, 5 or 6 membered monocyclic heterocyclyl group containing 1 nitrogen heteroatom. Suitable values of such groups represented by Q^{a} include the appropriate non-aromatic heterocyclyl groups listed above, more particularly azetidinyl, 1,3-thiazolidinyl, pyrrolidinyl, piperidinyl, piperazinyl, homopiperidinyl or homopiperazinyl (all of which are linked to X¹ in Formula I by a ring nitrogen). More particularly Q^{a} is selected from azetidin-1-yl, pyrrolidin-1-yl, piperidino 1,3-thiazolidin-3-yl, morpholino and piperazin-1-yl. Still more particularly Q^{a} is selected from azetidin-1-yl, pyrrolidin-1-yl, piperidino 1,3-thiazolidin-3-yl and morpholino. It is preferred that Q^{a} is selected from azetidin-1-yl, pyrrolidin-1-yl, piperidino and morpholino. More preferably Q^{a} is selected from pyrrolidin-1-yl, piperidino and morpholino. It is especially preferred that Q^{a} is pyrrolidin-1-yl.

Suitable values for any of the various groups within Formula I as defined hereinbefore or hereafter in this specification include:—
for halogeno fluoro, chloro, bromo and iodo;
for (1-6C)alkyl: methyl, ethyl, propyl, isopropyl, tert-butyl, pentyl and hexyl;
for (1-4C)alkyl: methyl, ethyl, propyl, isopropyl and tert-butyl;
for (1-6C)alkoxy: methoxy, ethoxy, propoxy, isopropoxy and butoxy;
for (2-8C)alkenyl: vinyl, isopropenyl, allyl and but-2-enyl;
for (2-8C)alkynyl: ethynyl, 2-propynyl and but-2-ynyl;
for (2-6C)alkenyloxy: vinyloxy and allyloxy;
for (2-6C)alkynyloxy: ethynyloxy and 2-propynyloxy;
for (1-6C)alkylthio: methylthio, ethylthio and propylthio;
for (2-6C)alkenylthio: vinylthio and allylthio;
for (2-6C)alkynylthio: ethynlythio and 2-propynylthio
for (1-6C)alkylsulfinyl: methylsulfinyl and ethylsulfinyl;
for (2-6C)alkenylsulfinyl: vinylsulfinyl and allylsulfinyl;
for (2-6C)alkynylsulfinyl: ethynylsulfinyl and 2-propynylsulfinyl
for (1-6C)alkylsulfonyl: methylsulfonyl and ethylsulfonyl;
for (2-6C)alkenylsulfonyl: vinylsulfonyl and allylsulfonyl;
for (2-6C)alkynylsulfonyl: ethynylsulfonyl and 2-propynylsulfonyl;
for (1-6C)alkylamino: methylamino, ethylamino, propylamino, isopropylamino and butylamino;
for di-[(1-6C)alkyl]amino: dimethylamino, diethylamino, N-ethyl-N-methylamino and diisopropylamino;
for (1-6C)alkoxycarbonyl: methoxycarbonyl, ethoxycarbonyl, propoxycarbonyl and tert-butoxycarbonyl;
for N-(1-6C)alkylcarbamoyl: N-methylcarbamoyl, N-ethylcarbamoyl, N-propylcarbamoyl and N-isopropylcarbamoyl;
for N,N-di-[(1-6C)alkyl]carbamoyl: N,N-dimethylcarbamoyl, N-ethyl-N-methylcarbamoyl and N,N-diethylcarbamoyl;
for (2-6C)alkanoyl: acetyl, propionyl and isobutyryl;
for (2-6C)alkanoyloxy: acetoxy and propionyloxy;
for (2-6C)alkanoylamino: acetamido and propionamido;
for N-(1-6C)alkyl-(2-6C)alkanoylamino: N-methylacetamido and N-methylpropionamido;
for N-(1-6C)alkylsulfamoyl: N-methylsulfamoyl, N-ethylsulfamoyl and N-isopropylsulfamoyl;
for N,N-di-[(1-6C)alkyl]sulfamoyl: N,N-dimethylsulfamoyl and N-methyl-N-ethylsulfamoyl;
for (1-6C)alkanesulfonylamino: methanesulfonylamino and ethanesulfonylamino;
for N-(1-6C)alkyl-(1-6C)alkanesulfonylamino: N-methylmethanesulfonylamino and N-methylethanesulfonylamino;
for amino-(1-6C)alkyl: aminomethyl, 2-aminoethyl, 1-aminoethyl and 3-aminopropyl;
for (1-6C)alkylamino-(1-6C)alkyl: methylaminomethyl, ethylaminomethyl, 1-methylaminoethyl, 2-methylaminoethyl, 2-ethylaminoethyl and 3-methylaminopropyl;
for di-[(1-6C)alkyl]amino-(1-6C)alkyl: dimethylaminomethyl, diethylaminomethyl, 1-dimethylaminoethyl, 2-dimethylaminoethyl and 3-dimethylaminopropyl;
for halogeno-(1-6C)alkyl: chloromethyl, 2-chloroethyl, 1-chloroethyl and 3-chloropropyl;
for hydroxy-(1-6C)alkyl: hydroxymethyl, 2-hydroxyethyl, 1-hydroxyethyl and 3-hydroxypropyl;
for hydroxy-(1-6C)alkoxy: hydroxymethoxy, 2-hydroxyethoxy, 1-hydroxyethoxy and 3-hydroxypropoxy;
for (1-6C)alkoxy-(1-6C)alkyl: methoxymethyl, ethoxymethyl, 1-methoxyethyl, 2-methoxyethyl, 2-ethoxyethyl and 3-methoxypropyl;
for cyano-(1-6C)alkyl: cyanomethyl, 2-cyanoethyl, 1-cyanoethyl and 3-cyanopropyl;
for amino(2-6C)alkanoyl: aminoacetyl and 2-aminopropionyl;
for (1-6C)alkylamino-(2-6C)alkanoyl: methylaminoacetyl and 3-(methylamino)propionyl;
for N,N-di-[(1-6C)alkyl]amino-(2-6C)alkanoyl: di-methylaminoacetyl and 3-(di-methylamino)propionyl;
for (2-6C)alkanoylamino-(1-6C)alkyl: acetamidomethyl, propionamidomethyl and 2-acetamidoethyl;
for N-(1-6C)alkyl-(2-6C)alkanoylamino(1-6C)alkyl: N-methylacetamidomethyl, N-methylpropionamidomethyl, 2-(N-methylacetamido)ethyl and 2-(N-methylpropionamido)ethyl;
for (1-6C)alkoxycarbonylamino-(1-6C)alkyl: methoxycarbonylaminomethyl, ethoxycarbonylaminomethyl, tert-butoxycarbonylaminomethyl and 2-methoxycarbonylaminoethyl;
for carbamoyl(1-6C)alkyl: carbamoylmethyl, 1-carbamoylethyl, 2-carbamoylethyl and 3-carbamoylpropyl;
for N-(1-6C)alkylcarbamoyl(1-6C)alkyl: N-methylcarbamoylmethyl, N-ethylcarbamoylmethyl, N-propylcarbamoylmethyl, 1-methylcarbamoyl)ethyl, 2-(N-methylcarbamoyl)ethyl and 3-(N-methylcarbamoyl)propyl;
for N,N di-(1-6C)alkylcarbamoyl(1-6C)alkyl: N,N-dimethylcarbamoylmethyl, N,N-diethylcarbamoylmethyl, N methyl, N-ethylcarbamoylmethyl, 1-(N,N-dimethylcarbamoyl)ethyl, 1-(N,N-diethylcarbamoyl)ethyl, 2-(N,N-dimethylcarbamoyl)ethyl, 2-(N-diethylcarbamoyl)ethyl and 3-(N,N-dimethylcarbamoyl)propyl;
for sulfamoyl(1-6C)alkyl: sulfamoylmethyl, 1-sulfamoylethyl, 2-sulfamoylethyl and 3-sulfamoylpropyl;
for N-(1-6C)alkylsulfamoyl(1-6C)alkyl: N-methylsulfamoylmethyl, N-ethylsulfamoylmethyl, N-propylsulfamoylmethyl, 1-(N-methylsulfamoyl)ethyl, 2-(N-methylsulfamoyl)ethyl and 3-(N-methylsulfamoyl)propyl;
for N,N di-(1-6C)alkylsulfamoyl(1-6C)alkyl: N,N-dimethylsulfamoylmethyl, N,N-diethylsulfamoylmethyl, N methyl, N-ethylsulfamoylmethyl, 1-(N,N-dimethylsulfamoyl)ethyl, 1-(N,N-diethylsulfamoyl)ethyl, 2-(N,N-dimethylsulfamoyl)ethyl, 2-(N,N-diethylsulfamoyl)ethyl and 3-(N,N-dimethylsulfamoyl)propyl;
for (2-6C)alkanoyl(1-6C)alkyl: acetylmethyl, propionylmethyl, 2-acetylethyl and 2-propionylethyl;
for (2-6C)alkanoyloxy(1-6C)alkyl: acetoxymethyl, propionyloxymethyl, 2-acetoxyethyl and 3-acetoxypropyl;
for (1-6C)alkoxy(1-6C)alkylS(O)_{q}: 2-methoxyethylsulfonyl, 2-methoxyethylsulpinyl and 2-methoxyethylthio;
for amino(1-6C)alkylS(O)_{q}: 2-aminoethylsulfonyl, 2-aminoethylsulfinyl and 2-aminoethylthio;
for N-(1-6C)alkylamino(1-6C)alkylS(O)_{q}: 2-(methylamino)ethylsulfonyl, 2-(ethylamino)ethylsulfinyl and 2-(methylamino)ethylthio; and
for N,N-di[(1-6C)alkyl]amino(1-6C)alkylS(O)_{q}: 2-(dimethylamino)ethylsulfonyl, 3-(dimethlyamino)propylsulfonyl, 2-(di-ethylamino)ethylsulfinyl and 2-(N-methyl-N-ethylamino)ethylthio.

It is to be understood that when, R¹ is a group (1-6C)alkoxy substituted by, for example amino to give for example a 2-aminoethoxy group, it is the (1-6C)alkoxy group that is attached to the quinazoline ring. An analogous convention applies to the other groups defined herein.

As defined hereinbefore, adjacent carbon atoms in any (2-6C)alkylene chain within, for example, a R¹ substituent may be optionally separated by the insertion into the chain of a group such as O, CON(R⁴), N(R⁴) or C≡C. For example, insertion of a C≡C group into the ethylene chain within a 2-morpholinoethoxy group gives rise to a 4-morpholinobut-2-ynyloxy group and, for example, insertion of a CONH group into the ethylene chain within a 3-methoxypropoxy group gives rise to, for example, a 2-(2-methoxyacetamido)ethoxy group. It is to be understood that the term (2-6C)alkylene chain refers to any CH₂CH₂ group (for example within R¹) and includes, for example alkylene chains within a (1-6C)alkyl, (1-6C)alkoxy, (2-8C)alkenyl, (2-8C)alkenyloxy, (2-8C)alkynyl and (2-8C)alkynyloxy group. For example the insertion of a N(CH₃) group between the third and fourth carbon atoms in a hex-5-enyloxy group in R¹ gives rise to a 3-(N-methyl-N-allylamino)propoxy group.

When, as defined hereinbefore, any CH₂═CH— or HC≡C— group within a R¹ substituent optionally bears at the terminal CH₂═ or HC≡ position a substituent such as a group of the formula Q⁴-X⁴— wherein X⁴ is, for example, NHCO and Q⁴ is a heterocyclyl-(1-6C)alkyl group, suitable R¹ substituents so formed include, for example, N-[heterocyclyl-(1-6C)alkyl]carbamoylvinyl groups such as N-(2-pyrrolidin-1-ylethyl)carbamoylvinyl or N-[heterocyclyl-(1-6C)alkyl]carbamoylethynyl groups such as N-(2-pyrrolidin-1-ylethyl)carbamoylethynyl.

When reference is made herein to any alkyl or alkylene groups optionally bearing one or more substituents, a CH₂ or CH₃ group within said alkyl or alkylene group optionally bears on each said CH₂ or CH₃ group one or more substituents. There are suitably 1 or 2 substituents present on each said CH₂ group and there are suitably 1, 2 or 3 such substituents present on each said CH₃ group. It is to be understood that the alkyl or alkylene groups which may be substituted include the carbon atoms within cycloalkyl rings and the carbon atoms within composite groups containing an alkyl or alklene chain, such as (1-6C)alkoxy groups. Suitable substituents so formed include, for example, hydroxy-substituted heterocyclyl-(1-6C)alkoxy groups such as 2-hydroxy-3-piperidinopropoxy and 2-hydroxy-3-morpholinopropoxy.

When reference is made herein to a group (for example a heterocyclyl group) optionally bearing “one or more” substituents the specified group suitably optionally bears 1, 2 or 3 substituents, which may be the same or different.

It is to be understood that when X² is CO, it is a carbonyl group. It is also to be understood that when X² is CH₂C(O) or CH₂SO₂, the CH₂ group is attached to Q^{a} and the carbonyl or sulfonyl group is attached to the nitrogen atom of the NR²⁰Z group in Formula I.

When in this specification reference is made to a (1-4C)alkyl group it is to be understood that such groups refer to alkyl groups containing up to 4 carbon atoms. A skilled person will realise that representative examples of such groups are those listed above under (1-6C)alkyl that contain up to 4 carbon atoms, such as methyl, ethyl, propyl, isopropyl, butyl and tert-butyl. Similarly, reference to a (1-3C)alkyl group refers to alkyl groups containing up to 3 carbon atoms such as methyl, ethyl, propyl and isopropyl. A from O, N(R⁴), CON(R⁴), N(R⁴)CO, CH═CH and C≡C, wherein R⁴ is hydrogen or (1-6C)alkyl,

and wherein any CH₂═CH— or HC≡C— group within a R¹ substituent optionally bears at the terminal CH₂═ or HC≡ position a substituent selected from carbamoyl, N-(1-6C)alkylcarbamoyl, N,N-di-[(1-6C)alkyl]carbamoyl, amino-(1-6C)alkyl, (1-6C)alkylamino-(1-6C)alkyl and di-[(1-6C)alkyl]amino-(1-6C)alkyl

and wherein any alkyl or alkylene group within a R¹ substituent optionally bears one or more (for example 1, 2 or 3) substituents selected from halogeno, (1-6C)alkyl, hydroxy, amino, cyano, carbamoyl, (1-6C)alkoxy, (1-6C)alkylamino, di-[(1-6C)alkyl]amino, N-(1-6C)alkylcarbamoyl and N,N-di-[(1-6C)alkyl]carbamoyl, or from a group of the formula:

—X⁵-Q³

wherein X⁵ is a direct bond or is selected from O, N(R⁶), CON(R⁶), N(R⁶)CO and C(R⁶)₂O, wherein R⁶ is hydrogen or (1-6C)alkyl, and Q³ is heterocyclyl or heterocyclyl-(1-6C)alkyl,

and wherein any heterocyclyl group within a substituent on R¹ is a 4, 5, 6 or 7 membered non-aromatic saturated or partially saturated heterocyclyl group (preferably a 4, 5, 6 or 7 membered monocyclic non-aromatic heterocyclyl group),

and wherein any heterocyclyl group within a substituent on R¹ optionally bears 1, 2 or 3 substituents, which may be the same or different, selected from halogeno, trifluoromethyl, hydroxy, amino, carbamoyl, (1-6C)alkyl, (2-8C)alkenyl, (2-8C)alkynyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, or from a group of the formula:

—X⁶—R⁷

wherein X⁶ is a direct bond or is selected from O and N(R⁸), wherein R⁸ is hydrogen or (1-6C)alkyl, and R⁷ is halogeno-(1-6C)alkyl, hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl and di-[(1-6C)alkyl]amino-(1-6C)alkyl,

and wherein any heterocyclyl group within a substituent on R¹ optionally bears 1 or 2 oxo substituents.

In another embodiment of the invention R¹ is selected from hydrogen, hydroxy, (1-6C)alkoxy, or from a group of the formula: similar convention is adopted for the other groups listed above such as (1-4C)alkoxy, (2-4C)alkenyl, (2-4C)alkynyl and (2-4C)alkanoyl.

When two W groups form a (1-4C)alkylene bridge, preferably the alkylene bridge is attached to adjacent atoms in the Q^{a} ring. Examples of (1-4C)alkylene bridges that may be formed by two W groups include methylene (—CH₂—), ethylene (—CH₂CH₂—) and propylene (—CH₂CH₂CH₂—). When two W groups form a (1-4C)alkylene bridge, the group —X²NZR²⁰ may be on the ring Q^{a} or on a carbon of the (1-4C)alkylene bridge. For example when Q^{a} is pyrrolidin-1-yl or piperidino examples of groups which may be formed by two W groups forming a (1-4C)alkylene bridge on Q^{a} include:

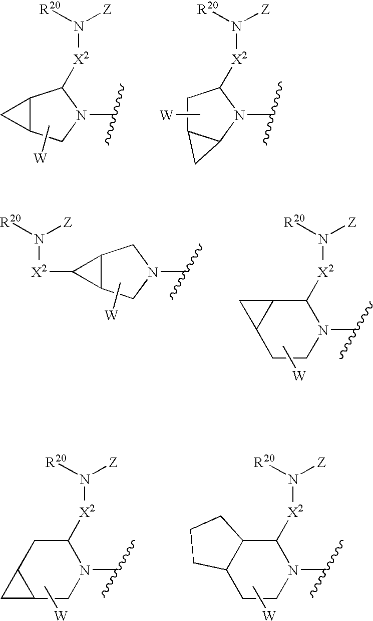

In the compound of Formula I hydrogen atoms are present at the 2, 5 and 8 positions on the quinazoline ring.

### Embodiments of R¹

In an embodiment of the invention R¹ is selected from hydrogen, hydroxy, (1-6C)alkoxy, (2-6C)alkenyloxy, (2-6C)alkynyloxy, or from a group of the formula:

Q¹-X³—

wherein X³ is O, and Q¹ is heterocyclyl or heterocyclyl-(1-6C)alkyl,

and wherein adjacent carbon atoms in any (2-6C)alkylene chain within a R¹ substituent are optionally separated by the insertion into the chain of a group selected

Q¹-X³—

wherein X³ is O, and Q¹ is (3-7C)cycloalkyl, (3-7C)cycloalkyl-(1-6C)alkyl, heterocyclyl or heterocyclyl-(1-6C)alkyl,

and wherein any heterocyclyl group within a substituent on R¹ is a 4, 5 or 6 membered monocyclic saturated or partially saturated heterocyclyl group,

and wherein adjacent carbon atoms in any (2-6C)alkylene chain within a R¹ substituent are optionally separated by the insertion into the chain of a group selected from O and N(R⁴), wherein R⁴ is hydrogen or (1-6C)alkyl,

and wherein any alkyl or alkylene group within a R¹ substituent optionally bears one or more substituents selected from halogeno, (1-6C)alkyl, hydroxy, amino, cyano, (1-6C)alkoxy, (1-6C)alkylamino and di-[(1-6C)alkyl]amino,

and wherein any heterocyclyl group within a substituent on R¹ optionally bears 1, 2 or 3 substituents, which may be the same or different, selected from halogeno, trifluoromethyl, cyano, nitro, hydroxy, amino, carbamoyl, (1-6C)alkyl, (2-8C)alkenyl, (2-8C)alkynyl, (1-6C)alkoxy, (1-6C)alkylsulfonyl, (1-6C)alkylamino, di-[(1-6C)alkyl]amino, N-(1-6C)alkylcarbamoyl, N,N-di-[(1-6C)alkyl]carbamoyl and (2-6C)alkanoyl,

and wherein any heterocyclyl group within a substituent on R¹ optionally bears 1 or 2 oxo substituents.

In another embodiment R¹ is selected from hydroxy, (1-6C)alkoxy, or from a group of the formula:

Q¹-X³—

wherein X³ is O, and Q¹ is azetidin-3-yl-(1-4C)alkyl, azetidin-1-yl-(2-4C)alkyl, pyrrolidin-2-yl-(1-4C)alkyl, pyrrolidin-3-yl-(1-4C)alkyl, pyrrolidin-1-yl-(2-4C)alkyl, piperidin-2-yl-(1-4C)alkyl, piperidin-3-yl-(1-4C)alkyl, piperidin-4-yl-(1-4C)alkyl, piperidino-(2-4C)alkyl, piperazino-(2-4C)alkyl or morpholino-(2-4C)alkyl,

and wherein adjacent carbon atoms in any (2-6C)alkylene chain within a R¹ substituent are optionally separated by the insertion into the chain of a group selected from O and N(R⁴), wherein R⁴ is hydrogen or (1-4C)alkyl,

and wherein any alkyl or alkylene group within a R¹ substituent optionally bears one or more substituents selected from fluoro, chloro, hydroxy, (1-4C)alkoxy, amino, (1-4C)alkylamino and di-[(1-4C)alkyl]amino,

and wherein any heterocyclyl group within a substituent on R¹ optionally bears 1, 2 or 3 substituents, which may be the same or different, selected from halogeno, hydroxy, amino, carbamoyl, (1-4C)alkyl, (2-4C)alkenyl, (2-4C)alkyl, (1-4C)alkoxy, (1-4C)alkylsulfonyl, (1-4C)alkylamino, di-[(1-4C)alkyl]amino, N-(1-4C)alkylcarbamoyl, N,N-di-(1-4C)alkyl]carbamoyl and (2-4C)alkanoyl,

and wherein any heterocyclyl group within a substituent on R¹ optionally bears 1 oxo substituent (preferably said oxo substituent is not on a carbon atom adjacent to a ring oxygen in the heterocyclyl group);

In a further embodiment of the invention R¹ is selected from hydroxy, (1-4C)alkoxy, hydroxy-(2-4C)alkoxy, (1-3C)alkoxy-(2-4C)alkoxy or from a group of the formula:

Q¹-X³—

wherein X³ is O, and Q¹ is azetidin-1-yl-(2-4C)alkyl, pyrrolidin-1-yl-(2-4C)alkyl, piperidino-(2-4C)alkyl, piperazino-(2-4C)alkyl or morpholino-(2-4C)alkyl,

and wherein any heterocyclyl group within a substituent on R¹ optionally bears 1, 2 or 3 substituents, which may be the same or different, selected from halogeno, hydroxy, amino, (1-4C)alkyl, (1-4C)alkoxy, (1-4C)alkylsulfonyl, (1-4C)alkylamino, di-[(1-4C)alkyl]amino, and (2-4C)alkanoyl,

and wherein any heterocyclyl group within a substituent on R¹ optionally bears 1 oxo substituent (preferably said oxo substituent is not on a carbon atom adjacent to a ring oxygen in the heterocyclyl group).

In a further embodiment of the invention R¹ is selected from (1-4C)alkoxy, hydroxy-(2-4C)alkoxy and (1-3C)alkoxy-(2-4C)alkoxy. More particularly R¹ is selected from (1-4C)alkoxy, hydroxy-(2-4C)alkoxy and (1-3C)alkoxy-(2-4C)alkoxy.

In a further embodiment of the invention, R¹ is selected from hydrogen, hydroxy, methoxy, ethoxy, propoxy, isopropyloxy, 2-hydroxyethoxy, 2-fluoroethoxy, cyclopropylmethoxy, 2-cyclopropylethoxy, vinyloxy, allyloxy, ethynyloxy, 2-propynyloxy, tetrahydrofuran-3-yloxy, tetrahydropyran-3-yloxy, tetrahydropyran-4-yloxy, tetrahydrofurfuryloxy, tetrahydrofuran-3-ylmethoxy, 2-(tetrahydrofuran-2-yl)ethoxy, 3-(tetrahydrofuran-2-yl)propoxy, 2-(tetrahydrofuran-3-yl)ethoxy, 3-(tetrahydrofuran-3-yl)propoxy, tetrahydropyranylmethoxy, 2-tetrahydropyranylethoxy, 3-tetrahydropyranylpropoxy, 2-pyrrolidin-1-ylethoxy, 3-pyrrolidin-1-ylpropoxy, pyrrolidin-3-yloxy, pyrrolidin-2-ylmethoxy, 2-pyrrolidin-2-ylethoxy, 3-pyrrolidin-2-ylpropoxy, 2-morpholinoethoxy, 3-morpholinopropoxy, 2-(1,1-dioxotetrahydro-4H-1,4-thiazinyl)ethoxy, 3-(1,1-dioxotetrahydro-4H-1,4-thiazin-4-yl)propoxy, 2-piperidinoethoxy, 3-piperidinopropoxy, piperidin-3-yloxy, piperidin-4-yloxy, piperidin-3-ylmethoxy, 2-piperidin-3-ylethoxy, piperidin-4-ylmethoxy, 2-piperidin-4-ylethoxy, 2-homopiperidin-1-ylethoxy, 3-homopiperidin-1-ylpropoxy, 2-piperazin-1-ylethoxy, 3-piperazin-1-ylpropoxy, 2-homopiperazin-1-ylethoxy, 3-homopiperazin-1-ylpropoxy, pyrrolidin-1-yl, morpholino, piperidino and piperazin-1-yl,

and wherein adjacent carbon atoms in any (2-6C)alkylene chain within a R¹ substituent are optionally separated by the insertion into the chain of a group selected from O, NH, N(CH₃), CH═CH and C≡C,

and wherein any CH₂ group which is attached to 2 carbon atoms or any CH₃ group which is attached to a carbon atom within an alkyl or alkylene group within a R¹ substituent optionally bears on each said CH₂ or CH₃ group 1, 2 or 3 fluoro substituents or a substituent selected from hydroxy, amino, methoxy, ethoxy, methylsulfonyl, methylamino and dimethylamino,

and wherein any heterocyclyl group within a substituent on R¹ optionally bears 1 or 2 substituents, which may be the same or different, selected from fluoro, chloro, trifluoromethyl, hydroxy, amino, methylamino, ethylamino, dimethylamino, diethylamino, carbamoyl, methyl, ethyl, n-propyl, isopropyl and methoxy, and any piperidin-3-ylmethyl, piperidin-4-ylmethyl, 2-piperazin-1-ylethyl, 3-piperazin-1-ylpropyl, or piperazin-1-yl group within a R¹ substituent is optionally N-substituted with 2-methoxyethyl, 3-methoxypropyl, 2-aminoethyl, 3-aminopropyl, 2-methylaminoethyl, 3-methylaminopropyl, 2-dimethylaminoethyl, 3-dimethylaminopropyl, acetyl or propionyl,

and wherein any heterocyclyl group within a substituent on R¹ optionally bears 1 oxo substituent (preferably said oxo substituent is not on a carbon atom adjacent to a ring oxygen in the heterocyclyl group, more preferably any heterocyclic group in R¹ does not carry an oxo substituent).

In another embodiment R¹ is selected from methoxy, ethoxy, propyloxy, isopropyloxy, cyclopropylmethoxy, 2-hydroxyethoxy, 2-fluoroethoxy, 2-methoxyethoxy, 2-ethoxyethoxy, 2,2-difluoroethoxy 2,2,2-trifluoroethoxy, 2-(pyrrolidin-1-yl)ethyl, 3-(pyrrolidin-1-yl)propyl, 2-piperidinoethyl, 3-piperidinopropyl, 2-piperazinoethyl, 3-piperazinopropyl, 2-morpholinoethyl and 3-morpholinopropyl.

In another embodiment R¹ is hydrogen, hydroxy, (1-6C)alkoxy, (2-6C)alkenyloxy, (2-6C)alkynyloxy, or a group of the formula:

Q¹-X³—

wherein X³ is O, and Q¹ is (3-7C)cycloalkyl, (3-7C)cycloalkyl-(1-6C)alkyl, (3-7C)cycloalkenyl, (3-7C)cycloalkenyl-(1-6C)alkyl, heterocyclyl or heterocyclyl-(1-6C)alkyl,

and wherein any alkyl or alkylene group within a R¹ substituent optionally bears one or more halogeno, (1-6C)alkyl, hydroxy, cyano, amino, carboxy, carbamoyl, sulfamoyl, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino.

In particular R¹ is selected from hydrogen, (1-6C)alkoxy and (1-6C)alkoxy(1-6C)alkoxy, wherein any (1-6C)alkoxy group in R¹ optionally bears one or more hydroxy substituents (suitably 1 or 2) and/or a substituent selected from amino, (1-4C)alkylamino, di-[(1-4C)alkyl]amino, carbamoyl, N-(1-4C)alkylcarbamoyl and N,N-di-[(1-4C)alkyl]carbamoyl, sulfamoyl, N-(1-4C)alkylsulfamoyl and N,N-di-[(1-4C)alkyl]sulfamoyl.

For instance, R¹ is selected from hydrogen, (1-6C)alkoxy and (1-4C)alkoxy(1-6C)alkoxy, and wherein any (1-6C)alkoxy group within R¹ optionally bears 1, 2 or 3 substituents, which may be the same or different, selected from hydroxy, fluoro and chloro, for example R¹ is selected from methoxy, ethoxy, isopropyloxy, cyclopropylmethoxy, 2-hydroxyethoxy, 2-fluoroethoxy, 2-methoxyethoxy, 2,2-difluoroethoxy, 2,2,2-trifluoroethoxy or 3-hydroxy-3-methylbutoxy.

In particular R¹ is selected from hydrogen, (1-4C)alkoxy and (1-4C)alkoxy(2-4C)alkoxy, more particularly R¹ is selected from (1-4C)alkoxy, hydroxy(2-4C)alkoxy and (1-3C)alkoxy(2-4C)alkoxy, more particularly R¹ is selected from (1-3C)alkoxy and (1-3C)alkoxy(2-3C)alkoxy. Preferably R¹ is (1-3C)alkoxy. For instance, R¹ is selected from hydrogen, methoxy, ethoxy and 2-methoxyethoxy or 2-hydroxyethoxy. A particular example of a group R¹ is methoxy.

### Embodiments of X¹

In one embodiment X¹ is (C(R⁹)₂)ₙ, wherein each R⁹, which may be the same or different, is selected from hydrogen, (1-4C)alkyl, halo(1-4C)alkyl, hydroxy (1-4C)alkyl, (1-4C)alkoxy(1-4C)alkyl, (3-6C)cycloalkyl and (3-6C)cycloalkyl-(1-2C)alkyl, or two groups R⁹ together with the carbon atom(s) to which they are attached form a (3-6C)cycloalkyl ring, and n is 1 or 2 (preferably 1). Preferably in this embodiment one R⁹ is hydrogen.

In another embodiment X¹ is C(R⁹)₂, wherein one R⁹ is hydrogen and the other R⁹ is selected from hydrogen, (1-4C)alkyl, cyclopropyl and cyclopropylmethyl, or the two groups R⁹ together with the carbon atom to which they are attached form a (3-6C)cycloalkyl ring (for example a cyclopropyl ring).

Suitably X¹ is (C(R⁹)₂)ₙ, wherein n is 1 or 2 and each R⁹, which may be the same or different, is selected from hydrogen, (1-4C)alkyl, hydroxymethyl, hydroxyethyl or halo(1-2)alkyl, such as CH₂CH₂F, CH₂CHF₂ or CH₂CF₃. In a particular embodiment each R⁹, which may be the same or different is selected from hydrogen and (1-4C)alkyl.

Where two groups R⁹ together with the carbon atom(s) to which they are attached form a (3-7C) cycloalkyl ring, it is preferably that both R⁹ groups are on the same carbon atom. Thus particular examples of such a group include cyclopropyl, cyclopentyl or cyclohexyl, particularly cyclopropyl.

In particular, at least one, and preferably each R⁹ is hydrogen.

Suitably n is 1.

Accordingly in a particular embodiment X¹ is CHR⁹, wherein R⁹ is selected from hydrogen, (1-4C) alkyl, hydroxy-(1-4C) alkyl (1-3C)alkoxy-(1-3C)alkyl.

In another particular embodiment X¹ is CHR⁹, wherein R⁹ is selected from hydrogen and (1-4C) alkyl (for example R⁹ is selected from hydrogen, methyl, ethyl and isopropyl, particularly R⁹ is hydrogen or methyl). It is preferred that X¹ is CH₂.

### Embodiments of W

In one embodiment each W, which may be the same or different is selected from halogeno, trifluoromethyl, cyano, nitro, hydroxy, oxo, amino, carbamoyl, sulfamoyl, formyl, mercapto, (1-6C)alkyl, (2-6C)alkenyl, (2-6C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino, and N-(1-6C)alkyl-(1-6C)alkanesulfonylamino, or a group of the formula:

—X⁷—R¹⁰

wherein X⁷ is a direct bond or is selected from O, CO and N(R¹¹), wherein R¹¹ is hydrogen or (1-6C)alkyl, and R¹⁰ is halogeno-(1-6C)alkyl, hydroxy-(1-6C)alkyl, (1-6C)alkoxy-(1-6C)alkyl, cyano-(1-6C)alkyl, amino-(1-6C)alkyl, N-(1-6C)alkylamino-(1-6C)alkyl, N,N-di-[(1-6C)alkyl]amino-(1-6C)alkyl, (2-6C)alkanoylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, (2-6C)alkanoyl-(1-6C)alkyl and (2-6C)alkanoyloxy-(1-6C)alkyl,

or two W groups form a (1-3C)alkylene bridge, which (1-3C)alkylene bridge optionally bears 1, 2 or 3 substituents, which may be the same or different, selected from halogeno, hydroxy, oxo, (1-4C)alkyl, (1-4C)alkoxy, amino, N-(1-4C)alkylamino and N,N-di-[(1-4C)alkyl]amino.

Suitably q is 0, 1, or 2. In particular q is 0. Alternatively q is 1.

Preferred groups W include halogeno, trifluoromethyl, cyano, nitro, hydroxy, oxo, amino, carbamoyl, sulfamoyl, formyl, mercapto, (1-6C)alkyl, (2-6C)alkenyl, (2-6C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino, and N-(1-6C)alkyl-(1-6C)alkanesulfonylamino, or a group of the formula:

—X⁷—R¹⁰

wherein X⁷ is a direct bond or is selected from O, CO and N(R¹¹), wherein R¹¹ is hydrogen or (1-6C)alkyl, and R¹⁰ is halogeno-(1-6C)alkyl, hydroxy-(1-6C)alkyl, (1-6C)alkoxy-(1-6C)alkyl, cyano-(1-6C)alkyl, amino-(1-6C)alkyl, N-(1-6C)alkylamino-(1-6C)alkyl, N,N-di-[(1-6C)alkyl]amino-(1-6C)alkyl, (2-6C)alkanoylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, (2-6C)alkanoyl-(1-6C)alkyl and (2-6C)alkanoyloxy-(1-6C)alkyl.

In another embodiment q is 0, 1, 2 or 3 (preferably 0 or 1, more preferably 0) and each W, which may be the same or different, is selected from hydroxy, amino, (1-4C)alkyl, (1-4C)alkoxy, (1-4C)alkylamino, di-[(1-4C)alkyl]amino, hydroxy-(1-4C)alkyl and (1-4C)alkoxy-(1-4C)alkyl,

or two W groups on adjacent ring carbon atoms in Q^{a} form a (1-3C)alkylene bridge, which (1-3C)alkylene bridge optionally bears 1 or 2 substituents, which may be the same or different, selected from halogeno, hydroxy, oxo, (1-3C)alkyl and (1-3C)alkoxy.

In another embodiment q is 0, 1 or 2 (preferably 0 or 1, more preferably 0) and each W, which may be the same or different, is selected from hydroxy, amino, (1-4C)alkyl, (1-4C)alkoxy, (1-4C)alkylamino, di-[(1-4C)alkyl]amino, hydroxy-(1-4C)alkyl and (1-4C)alkoxy-(1-4C)alkyl.

In another embodiment q is 0, 1 or 2 (preferably 0 or 1, more preferably 0) and each W, which may be the same or different, is selected from hydroxy, amino, (1-4C)alkyl, (1-4C)alkoxy, (1-4C)alkylamino, di-[(1-4C)alkyl]amino, hydroxy-(1-4C)alkyl and (1-4C)alkoxy-(1-4C)alkyl.

or two W groups on adjacent ring carbon atoms in Q^{a} form a (1-3C)alkylene bridge, which (1-3C)alkylene bridge optionally bears 1 or 2 substituents, which may be the same or different, selected from hydroxy, (1-3C)alkyl and (1-3C)alkoxy.

In another embodiment q is 0, 1, 2 or 3 (preferably 0 or 1, more preferably 0) and each W, which may be the same or different, is selected from hydroxy, (1-4C)alkyl, (1-4C)alkoxy, hydroxy-(1-4C)alkyl and (1-4C)alkoxy-(1-4C)alkyl,

or two W groups on adjacent ring carbon atoms in Q^{a} form a (1-3C)alkylene bridge.

In another embodiment q is 2 and the two W groups are on adjacent ring carbon atoms in Q^{a} and form a (1-3C)alkylene bridge, which (1-3C)alkylene bridge optionally bears 1 or 2 substituents, which may be the same or different, selected from hydroxy, (1-3C)alkyl and (1-3C)alkoxy, for example two W groups form a methylene bridge.

In another embodiment q is 0, 1 or 2 (preferably 0 or 1, more preferably 0) and each W, which may be the same or different, is selected from hydroxy, (1-4C)alkyl, (1-4C)alkoxy, hydroxy-(1-4C)alkyl and (1-4C)alkoxy-(1-4C)alkyl.

In another embodiment q is 0 or 1, more preferably 0 and W is selected from hydroxy and (1-4C)alkoxy.

Particular values of W are groups of formula —OR²², where R² is R²² is hydrogen, (1-6C)alkyl, (2-6C)alkenyl, (2-6C)alkynyl, (2-6C)alkanoyl, or a group R¹⁰ where R¹⁰ is as defined above in relation to Formula (I).

Particular examples of R²² include hydrogen, (1-6C)alkyl such as methyl, ethyl, propyl, n-butyl, halogeno-(1-6C)alkyl, hydroxy-(2-6C)alkyl or (1-6C)alkoxy-(2-6C)alkyl.

More particularly R²² is selected from (1-4C)alkyl such as methyl, ethyl, propyl, iso-propyl, n-butyl, halogeno-(1-6C)alkyl, hydroxy-(2-6C)alkyl or (1-6C)alkoxy-(2-6C)alkyl.

More specifically, R²² may be hydrogen or (1-6Calkyl). More particularly R²² is (1-4C)alkyl such as methyl.

In another embodiment q is 0, 1 or 2 (preferably 0 or 1) and each W, which may be the same or different, is selected from hydroxy, amino, methyl, ethyl, isopropyl, methoxy, ethoxy, isopropyloxy, methylamino, ethylamino, dimethylamino and diethylamino.

In another embodiment q is 0, 1 or 2 (preferably 0 or 1) and each W, which may be the same or different, is selected from hydroxy, (1-3C)alkyl, (1-3C)alkoxy, hydroxy-(1-4C)alkyl and (1-3C)alkoxy-(2-3C)alkyl. For example q is 0 or 1 and W is selected from methyl, ethyl, hydroxy, methoxy, ethoxy, 2-methoxyethyl and 2-hydroxyethyl. More particularly q is 0 or 1 and W is selected from methyl, ethyl, methoxy and ethoxy.

### Embodiments of X²

Suitably X² is selected from C(O), SO₂ and CH₂C(O). In a particular embodiment X² is C(O). In another embodiment X² is SO₂.

### Embodiments of R²⁰

In one embodiment R²⁰ is selected from hydrogen, (1-4C)alkyl and (1-3C)alkoxy-(2-4C)alkyl. More particularly R²⁰ is selected from hydrogen and (1-4C)alkyl. For example R²⁰ is hydrogen, methyl, ethyl or isopropyl.

Suitably R²⁰ is hydrogen, methyl, ethyl or propyl.

It is preferred that R²⁰ is hydrogen.

### Embodiments of Z

Z is selected from hydrogen, (1-6C)alkyl, (2-6C)alkenyl, (2-6C)alkynyl,

and wherein adjacent carbon atoms in any (2-6C)alkylene chain within a Z substituent are optionally separated by the insertion into the chain of a group selected from O, S, SO, SO₂, and CO,

and wherein any CH₂═CH— or HC≡C— group within a Z substituent optionally bears at the terminal CH₂═ or HC≡ position a substituent selected from halogeno, carboxy, carbamoyl,

and wherein any alkyl or alkylene group within a Z substituent, optionally bears on one or more halogeno or (1-6C)alkyl substituents or a substituent selected from hydroxy, cyano, amino, carboxy, carbamoyl, sulfamoyl, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino or (3-8C)cycloalkyl or heterocylyl, either of which may be optionally substituted by one or more groups selected from halogeno, cyano, nitro, hydroxy, amino, carboxy, carbamoyl, sulfamoyl, trifluoromethyl, (1-4C)alkyl, (2-4C)alkenyl, (2-4C)alkynyl, (1-3C)alkoxy, (2-4C)alkenyloxy, (2-4C)alkynyloxy, (1-4C)alkylthio, (1-4C)alkylsulfinyl, (1-4C)alkylsulfonyl, (1-4C)alkylamino, di-[(1-4C)alkyl]amino, (1-4C)alkoxycarbonyl.

In another embodiment, Z is selected from hydrogen, (1-6C)alkyl, (2-6C)alkenyl, (2-6C)alkynyl, (3-6C)cycloalkyl, (3-6C)cycloalkyl-(1-4C)alkyl, heteroaryl, heteroaryl-(1-4C)alkyl, azetidinyl, azetidinyl-(1-4C)alkyl, pyrrolinyl, pyrrolinyl-(1-4C)alkyl, pyrrolidinyl, pyrrolidinyl-(1-4C)alkyl, morpholinyl, morpholinyl-(1-4C)alkyl, piperidinyl, piperidinyl-(1-4C)alkyl, piperazinyl, piperazinyl-(1-4C)alkyl, phenyl and phenyl-(1-4C)alkyl,

and wherein any heteroaryl within Z is selected from isoxazolyl, furyl, thienyl, pyridyl, pyrazolyl, pyrrolyl, indolyl, quinolinyl, benzofuranyl and benzothienyl,

and wherein adjacent carbon atoms in any (2-6C)alkylene chain within a Z substituent are optionally separated by the insertion into the chain of a group selected from O and N(R¹²), wherein R¹² is selected from hydrogen and (1-3C)alkyl,

and wherein any alkyl, (3-6C)cycloalkyl or alkylene group within a Z substituent, optionally bears on one or more halogeno or (1-4C)alkyl substituents or a substituent selected from hydroxy, cyano, amino, (1-4C)alkoxy, (1-4C)alkylamino and di-[(1-4C)alkyl]amino,

and wherein any, phenyl, heteroaryl or heterocyclyl group within a Z substituent optionally bears one or more substituents selected from halogeno (particularly bromo, chloro or fluoro), amino, nitro, cyano, hydroxy, (1-4C)alkyl, (1-4C)alkoxy, (2-4C)alkanoyl, (1-4C)alkylsulfonyl, carbamoyl, [(1-4C)alkyl]amino, di-[(1-4C)alkyl]amino such as dimethylamino, N-[(1-4C)alkyl]carbamoyl and N,N-di[(1-4C)alkyl]carbamoyl such as N,N-dimethylcarbamoyl.

In another embodiment, Z is selected from hydrogen, (1-6C)alkyl, (2-6C)alkenyl, (2-6C)alkynyl, (3-6C)cycloalkyl, (3-6C)cycloalkyl-(1-4C)alkyl, heteroaryl, heteroaryl-(1-4C)alkyl, azetidinyl, azetidinyl-(1-4C)alkyl, pyrrolinyl, pyrrolinyl-(1-4C)alkyl, pyrrolidinyl, pyrrolidinyl-(1-4C)alkyl, piperidinyl, piperidinyl-(1-4C)alkyl, piperazinyl and piperazinyl-(1-4C)alkyl,

and wherein any heteroaryl within Z is selected from isoxazolyl, furyl, thienyl, pyridyl, pyrazolyl, pyrrolyl, indolyl, quinolinyl, benzofuranyl and benzothienyl,

and wherein adjacent carbon atoms in any (2-6C)alkylene chain within a Z substituent are optionally separated by the insertion into the chain of a group selected from O, NH and N(Me),

and wherein any alkyl, (3-6C)cycloalkyl or alkylene group within a Z substituent, optionally bears on one or more halogeno or (1-4C)alkyl substituents or a substituent selected from hydroxy, cyano, amino, (1-4C)alkoxy, (1-4C)alkylamino and di-[(1-4C)alkyl]amino,

and wherein any, heteroaryl or heterocyclyl group within a Z substituent optionally bears one or more substituents selected from halogeno (particularly bromo, chloro or fluoro), amino, nitro, cyano, hydroxy, (1-4C)alkyl, (1-4C)alkoxy, (2-4C)alkaoyl, (1-4C)alkylsulfonyl, carbamoyl, [(1-4C)alkyl]amino, di-[(1-4C)alkyl]amino such as dimethylamino, N-[(1-4C)alkyl]carbamoyl and N,N-di[(1-4C)alkyl]carbamoyl such as N,N-dimethylcarbamoyl,

and wherein any pyrrolinyl, pyrrolidinyl, piperidinyl or piperazinyl group within a Z substituent optionally bears 1 or 2 oxo substituents.

In another embodiment, Z is selected from hydrogen, (1-4C)alkyl, (2-4C)alkenyl, (2-4C)alkynyl, heteroaryl and heteroaryl-(1-4C)alkyl,

and wherein any heteroaryl within Z is selected from isoxazolyl, furyl, thienyl, pyridyl, pyrazolyl, pyrrolyl and indolyl,

and wherein any alkyl or alkylene group within a Z substituent, optionally bears on one or more halogeno (such as fluoro or chloro) or (1-4C)alkyl substituents or a substituent selected from hydroxy, cyano, amino, (1-4C)alkoxy, (1-4C)alkylamino and di-[(1-4C)alkyl]amino,

and wherein any, heteroaryl or heterocyclyl group within a Z substituent optionally bears one or more substituents selected from halogeno (particularly bromo, chloro or fluoro), amino, nitro, cyano, hydroxy, (1-4C)alkyl, (1-4C)alkoxy, [(1-4C)alkyl]amino and di-[(1-4C)alkyl]amino such as dimethylamino.

In another embodiment Z is selected from hydrogen, (1-4C)alkyl, (2-4C)alkenyl and (2-4C)alkynyl,

and wherein any alkyl or alkylene group within a Z substituent, optionally bears on one or more substituents selected from fluoro and chloro, or a substituent selected from hydroxy, cyano, amino, (1-3C)alkoxy, (1-3C)alkylamino and di-[(1-3C)alkyl]amino.

In another embodiment Z is selected from hydrogen, (1-4C)alkyl, (2-4C)alkenyl, (2-4C)alkynyl, hydroxy-(2-4C)alkyl, (1-3C)alkoxy-(2-4C)alkyl, cyano-(1-4C)alkyl, amino-(2-4C)alkyl, (1-3C)alkylamino-(2-4C)alkyl and di-[(1-3C)alkyl]amino-(2-4C)alkyl.

In another embodiment Z is selected from hydrogen, (1-3C)alkyl, (2-3C)alkenyl (2-3C)alkynyl, hydroxy-(2-3C)alkyl, (1-3C)alkoxy-(2-3C)alkyl and cyano-(1-3C)alkyl.

In particular, Z is selected from hydrogen, (1-6C)alkyl, (2-6C)alkenyl or (2-6C)alkynyl.

In another embodiment Z is selected from hydrogen, methyl, ethyl, isopropyl, allyl, 2-propynyl and cyanomethyl.

In a further embodiment Z is selected from hydrogen and (1-3C)alkyl (for example Z is selected from hydrogen, methyl and ethyl.

It is preferred that Z is hydrogen.

In another embodiment of the invention R²⁰ is hydrogen and Z is selected from hydrogen and (1-3C)alkyl. It is preferred that Z and R²⁰ are both hydrogen.

In another embodiment of the invention the group —X²NZR²⁰ is in the ortho (2-) position relative to the ring nitrogen atom in Q^{a} that is attached to X¹ in Formula I. More particularly the group —X²NZR²⁰ is —C(O)NZR²⁰ and is in the ortho (2-) position relative to the ring nitrogen atom in Q^{a} that is attached to X¹ in Formula I, wherein Z and R²⁰ have any of the values defined herein.

### Embodiments of the Aniline Group in Formula I

In an embodiment of the invention, a is 1, 2 or 3.

In a particular embodiment, when R³ is in the para position on the anilino ring it is selected from halogeno, cyano, nitro, hydroxy, amino, trifluoromethyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylamino and di-[(1-6C)alkyl]amino.

Examples of suitable R³ substituents are halogeno, carbamoyl, trifluoromethyl, (1-6C)alkyl, (2-8C)alkenyl, (2-8C)alkynyl, N-(1-6C)alkylcarbamoyl, or N,N-di-[(1-6C)alkyl]carbamoyl. In a particular embodiment at least one R³, and suitably all R³ groups are halogeno, such as chloro or fluoro.

Particular examples of the group of sub-formula (I):

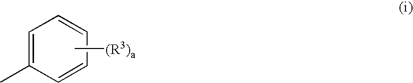

in Formula I are groups of sub-formula (II):

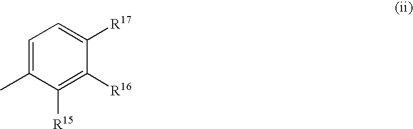

wherein one of R¹⁵ or R¹⁷ is hydrogen and the other is halogeno, such as chloro or fluoro, and preferably fluoro, and R¹⁶ is halogeno such as bromo, chloro or fluoro, particularly chloro or fluoro and still more particularly chloro or bromo. Preferably R¹⁶ is chloro.

Particular examples of such groups are 3-chloro-2-fluorophenyl, 3-bromo-2-fluorophenyl or 3-chloro-4-fluorophenyl.

In a further particular embodiment, a is 1 or 2. In one embodiment a is 1. In a further embodiment a is 2, one R³ is fluoro and the other is chloro or bromo.

In another embodiment a is 1 or 2 and each R³, which may be the same or different, is selected from fluoro, chloro, bromo and ethynyl. In this embodiment it is preferred that one R³ is in the meta (3-) position on the anilino group in Formula I and is selected from chloro, bromo and ethynyl (preferably chloro or bromo) and when a is 2, the other R³ is in the ortho (2-) position and is fluoro. Preferably when a is 1 R³ is in the meta (3-) position on the anilino group in Formula I and is bromo or ethynyl.

In another embodiment the anilino group at the 4-position on the quinazoline ring in Formula I is selected from 3-chloro-4-fluoroanilino, 3-bromo-2-fluoroanilino, 3-chloro-2-fluoroanilino, 2-fluoro-5-chloroanilino, 3-bromoanilino and 3-ethynylanilino.

More particularly the anilino group at the 4-position on the quinazoline ring in Formula I is selected from 3-chloro-4-fluoroanilino, 3-bromo-2-fluoroanilino and 3-chloro-2-fluoroanilino. Still more particularly the anilino group is 3-bromo-2-fluoroanilino or, preferably, 3-chloro-2-fluoroanilino.

### Embodiments of X⁸ in Formula IA

In one embodiment X⁸ is selected from CH₂, O or NR¹³. Where X⁸ is a group of formula NR¹³, wherein R¹³ is hydrogen, carbamoyl, sulfamoyl, formyl, (1-6C)alkyl, (2-6C)alkenyl, (2-6C)alkynyl, (1-6C)alkylsulfonyl, N-(1-6C)alkylcarbamoyl, N,N-di-[(1-6C)alkyl]carbamoyl, N-(1-6C)alkylsulfamoyl, and N,N-di-[(1-6C)alkyl]sulfamoyl, or from a group of the formula:

—X⁷—R¹⁰

wherein X⁷ is a direct bond or is CO and R¹⁰ is as defined above, for example R¹⁰ is (1-6C)alkyl optionally substituted by halogeno, hydroxy, (1-6C)alkoxy, amino, (1-4C)alkylamino and N,N-di-[(1-6C)alkyl]amino.

In one embodiment X⁸ is selected from CH₂, O or NR¹³. Where X⁸ is a group of formula NR¹³, particular examples of the group R¹³ include hydrogen, carboxy, carbamoyl, N-(1-6C)alkylcarbamoyl, N,N-di-[(1-6C)alkyl]carbamoyl, (2-6C)alkanoyl, or a group of the formula:

—X⁷—R¹⁰

where X⁷ and R¹⁰ are as defined above.

In particular, in this case, X⁷ is a direct bond or a C(O) group. R¹⁰ is suitably selected from (1-6C)alkyl optionally substituted by one or more groups, for example from 1 to 3 groups, selected from halogeno, hydroxy or (1-6C)alkoxy. Examples of such groups —X⁷—R¹⁰ include CH₃; COCH₃; COCH₂OH; COCH₂OCH₃; COCH(OH)CH₂OH; COCH(OCH₃)CH₂(OCH₃); COCH(OH)CH₂OCH₃; COCH(OCH₃)CH₂OH; COCH₂CH₂OCH₃; COCH₂CH₃; COCH(OH)CH₃; or COCH(OCH₃)CH₃.

In one embodiment Xs is NR¹³ wherein R¹³ is selected from hydrogen, (1-4C)alkyl, hydroxy-(2-4C)alkyl and (1-3C)alkoxy-(2-4C)alkyl. For example R¹³ is selected from hydrogen, methyl, ethyl and 2-methoxyethyl.

Suitably R¹³ is hydrogen or methyl.

In a particular embodiment X⁸ in Formula IA is O or CH₂.

In a particular embodiment however, b in Formula IA is 0.

### Particular Embodiments of Formula I

In a preferred embodiment, in the quinazoline of Formula I the group —X²ZR²⁰ is in the ortho (2-) position relative to the ring nitrogen atom in Q^{a} that is attached to X¹ in Formula I.

In an embodiment of the invention there is provided a quinazoline derivative of the Formula I as defined hereinbefore, wherein:

R¹ is selected from hydrogen, hydroxy, (1-4C)alkoxy, hydroxy-(2-4C)alkoxy and (1-3C)alkoxy-(2-4C)alkoxy (particularly R¹ is selected from (1-4C)alkoxy, hydroxy-(2-4C)alkoxy and (1-3C)alkoxy-(2-4C)alkoxy;

X¹ is C(R⁹)₂, wherein one R⁹ is hydrogen and the other R⁹ is selected from hydrogen, (1-4C) alkyl, hydroxy-(1-4C) alkyl (1-3C)alkoxy-(1-3C)alkyl (particularly R⁹ is hydrogen or (1-3C)alkyl, more particularly R⁹ is hydrogen),

or the two groups R⁹ together with the carbon atom to which they are attached form a (3-6C)cycloalkyl ring (for example a cyclopropyl ring);

Q^{a} is selected from azetidin-1-yl, pyrrolidin-1-yl, piperidino 1,3-thiazolidin-3-yl and morpholino;

q is 0, 1, 2 or 3 (particularly 0, 1 or 2);

each W, which may be the same or different, is selected from hydroxy, amino, (1-4C)alkyl, (1-4C)alkoxy, (1-4C)alkylamino, di-[(1-4C)alkyl]amino, hydroxy-(1-4C)alkyl and (1-4C)alkoxy-(1-4C)alkyl,

or two W groups on adjacent ring carbon atoms in Q^{a} form a (1-3C)alkylene bridge, which (1-3C)alkylene bridge optionally bears 1 or 2 substituents, which may be the same or different, selected from hydroxy, (1-3C)alkyl and (1-3C)alkoxy;

X² is selected from CH₂C(O) and C(O) (preferably X² is C(O));

Z is selected from hydrogen, (1-4C)alkyl, (2-4C)alkenyl, (2-4C)alkynyl, hydroxy-(2-4C)alkyl, (1-3C)alkoxy-(2-4C)alkyl, cyano-(1-4C)alkyl, amino-(2-4C)alkyl, (1-3C)alkylamino-(2-4C)alkyl and di-[(1-3C)alkyl]amino-(2-4C)alkyl;

R²⁰ is hydrogen;

a is 1, 2 or 3 (preferably a is 1 or 2);

each R³, which may be the same or different, is selected from fluoro, chloro, bromo and ethynyl;

or a pharmaceutically acceptable salt thereof.

In this embodiment it is preferred that the group —X²ZR²⁰ is in the ortho (2-) position relative to the ring nitrogen in Q^{a} atom that is attached to X¹ in Formula I.

In this embodiment a particular value for Z is a group selected from hydrogen (1-3C)alkyl, (2-3C)alkenyl and (2-3C)alkynyl. More particularly Z is selected from hydrogen, methyl and ethyl. It is preferred that Z is hydrogen.

In this embodiment a particular value for q is 0 or 1 and W is selected from hydroxy,(1-3C)alkyl, (1-3C)alkoxy, hydroxy-(1-3C)alkyl and (1-3C)alkoxy-(1-3C)alkyl.

In this embodiment it is preferred that a is 1 or 2 and that one R³ is in the meta (-3) position on the anilino group and is selected from chloro, bromo and ethynyl (particularly chloro or bromo), and any other R³ is in the ortho (2-) or para (4-) position on the anilino group and is selected from fluoro and chloro (particularly fluoro).

In this embodiment a particular anilino group at the 4-position on the quinazoline ring in Formula I is selected from 3-chloro-4-fluoroanrlino, 3-bromo-2-fluoroanilino, 3-chloro-2-fluoroanilino, 3-bromoanilino and 3-ethynylanilino. Still more particularly the anilino group is 3-bromo-2-fluoroanilino or, preferably, 3-chloro-2-fluoroanilino.

In another embodiment of the invention there is provided a quinazoline derivative of the Formula I of the Formula IB:

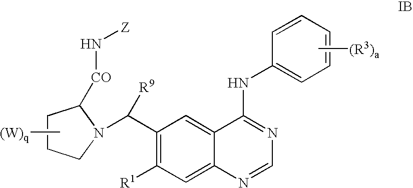

wherein R¹, R³, Z, W and q have any of the values defined herein in relation to Formula I;

R⁹ is selected from hydrogen and (1-3C)alkyl (for example R⁹ is hydrogen or methyl, preferably R⁹ is hydrogen); and

a is 1, 2 or 3 (preferably 1 or 2) and each R³, which may be the same or different is selected from fluoro, chloro, bromo and ethynyl (preferably one R³ is in the meta (3-) position on the anilino group in Formula IB and is selected from chloro, bromo and ethynyl and when a is 2 the other R³ is fluoro);

or a pharmaceutically acceptable salt thereof.

In the quinazoline derivative of Formula IB a particular value for R¹ is (1-4C)alkoxy, hydroxy-(2-4C)alkoxy and (1-3C)alkoxy-(2-4C)alkoxy. More particularly R¹ is (1-4C)alkoxy such as methoxy, ethoxy or isopropyloxy.

In the quinazoline derivative of Formula IB a particular value for q is 0 or 1 and W is selected from hydroxy, amino, (1-4C)alkyl, (1-4C)alkoxy, (1-4C)alkylamino, di-[(1-4C)alkyl]amino, hydroxy-(1-4C)alkyl and (1-4C)alkoxy-(1-4C)alkyl. Particularly W is selected from hydroxy, (1-3C)alkyl, (1-3C)alkoxy, hydroxy-(1-3C)alkyl and (1-3C)alkoxy-(1-3C)alkyl,

or q is 2 and the two W groups on adjacent ring carbon atoms in the pyrrolidin-1-yl ring form a (1-3C)alkylene bridge, which (1-3C)alkylene bridge optionally bears 1 or 2 substituents, which may be the same or different, selected from hydroxy,(1-3C)alkyl and (1-3C)alkoxy.

A particular value for Z in Formula IB is a group selected from hydrogen and (1-3C)alkyl. Preferably however, Z is hydrogen.

In this embodiment a particular anilino group at the 4-position on the quinazoline ring in Formula IB is selected from 3-chloro-4-fluoroanilino, 3-bromo-2-fluoroanilino, 3-chloro-2-fluoroanilino, 3-bromoanilino and 3-ethynylanilino. Still more particularly the anilino group is 3-bromo-2-fluoroanilino or, preferably, 3-chloro-2-fluoroanilino.

Accordingly a particular quinazoline derivative is of the Formula IB as hereinbefore defined wherein:

R¹ is (1-4C)alkoxy;

R⁹ is hydrogen or methyl (preferably hydrogen);

q is 0, 1 or 2 (preferably 0 or 1) and W has any of the values defined hereinbefore for W in relation to the quinazoline derivative of Formula I (particularly W is selected from hydroxy, (1-3C)alkyl, (1-3C)alkoxy, hydroxy-(1-3C)alkyl and (1-3C)alkoxy-(1-3C)alkyl, for example W is hydroxy, methoxy or ethoxy);

Z is selected from hydrogen and (1-3C)alkyl (preferably Z is hydrogen); and

the anilino group at the 4-position on the quinazoline ring is selected from 3-chloro-4-fluoroanilino, 3-bromo-2-fluoroanilino and particularly 3-chloro-2-fluoroanilino;

or a pharmaceutically acceptable salt thereof.

Another embodiment of the invention is a compound of Formula IA, wherein q is 1 and W is at the 4-position on the pyrrolidin-1-yl ring, so the quinazoline derivative of the Formula I is represented as Formula IC, or a pharmaceutically acceptable salt thereof:

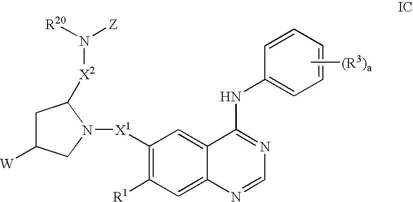

where W, R¹, R³, X¹, X², R²⁰ and Z are as defined herein in relation to Formula IA.

In the quinazoline derivative of Formula IC X² is suitably C(O).

In an embodiment of the invention there is provided a quinazoline derivative of Formula IC as hereinbefore defined, or a pharmaceutically acceptable salt thereof, wherein:

R¹ is (1-4C)alkoxy;

X¹ is CH₂ or CH(CH₃) (preferably X¹ is CH₂);

X² is C(O);

R²⁰ is hydrogen;

Z is hydrogen or (1-3C)alkyl;

W has any of the values defined hereinbefore for W in relation to the quinazoline derivative of Formula I; and

the anilino group at the 4-position in Formula IC is selected from 3-chloro-4-fluoroanilino, 3-bromo-2-fluoroanilino, 3-chloro-2-fluoroanilino, 3-bromoanilino and 3-ethynylanilino (particularly the anilino group is 3-bromo-2-fluoroanilino or, preferably, 3-chloro-2-fluoroanilino).

In this embodiment q is 0 or 1 and W is selected from hydroxy, amino, (1-4C)alkyl, (1-4C)alkoxy, (1-4C)alkylamino, di-[(1-4C)alkyl]amino, hydroxy-(1-4C)alkyl and (1-4C)alkoxy-(1-4C)alkyl. Particularly W is selected from hydroxy, (1-3C)alkyl, (1-3C)alkoxy, hydroxy-(1-3C)alkyl and (1-3C)alkoxy-(1-3C)alkyl, for example W is hydroxy, methoxy or ethoxy.

In another embodiment of the invention there is provided a quinazoline derivative of the Formula I of the Formula ID:

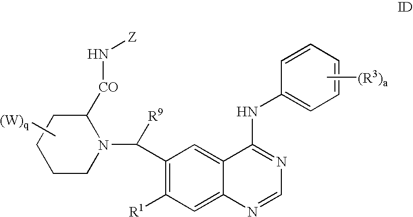

wherein R¹, R³, Z, W and q have any of the values defined herein in relation to Formula I;

R⁹ is selected from hydrogen and (1-3C)alkyl (for example R⁹ is hydrogen or methyl, preferably R⁹ is hydrogen); and

a is 1, 2 or 3 (preferably 1 or 2) and each R³, which may be the same or different is selected from fluoro, chloro, bromo and ethynyl;

or a pharmaceutically acceptable salt thereof.

In the quinazoline derivative of Formula ID a particular value for R¹ is (1-4C)alkoxy, hydroxy-(2-4C)alkoxy and (1-3C)alkoxy-(2-4C)alkoxy. More particularly R¹ is (1-4C)alkoxy such as methoxy, ethoxy or isopropyloxy.

In the quinazoline derivative of Formula ID a particular value for q is 0 or 1 and W is selected from hydroxy, amino, (1-4C)alkyl, (1-4C)alkoxy, (1-4C)alkylamino, di-[(1-4C)alkyl]amino, hydroxy-(1-4C)alkyl and (1-4C)alkoxy-(1-4C)alkyl. Particularly W is selected from hydroxy, (1-3C)alkyl, (1-3C)alkoxy, hydroxy-(1-3C)alkyl and (1-3C)alkoxy-(1-3C)alkyl.

A particular value for Z in Formula ID is a group selected from hydrogen and (1-3C)alkyl. Preferably however, Z is hydrogen.

In this embodiment a particular anilino group at the 4-position on the quinazoline ring in Formula ID is selected from 3-chloro-4-fluoroanilino, 3-bromo-2-fluoroanilino, 3-chloro-2-fluoroanilino, 3-bromoanilino and 3-ethynylanilino. Still more particularly the anilino group is 3-bromo-2-fluoroanilino or, preferably, 3-chloro-2-fluoroanilino.

Accordingly a particular quinazoline derivative is of the Formula ID as hereinbefore defined wherein:

R¹ is (1-4C)alkoxy;

R⁹ is selected from hydrogen and methyl (preferably methyl);

q is 0, 1 or 2 (preferably 0 or 1) and W has any of the values defined hereinbefore for W in relation to the quinazoline derivative of Formula I (particularly W is selected from hydroxy, (1-3C)alkyl, (1-3C)alkoxy, hydroxy-(1-3C)alkyl and (1-3C)alkoxy-(1-3C)alkyl, for example W is hydroxy, methoxy or ethoxy);

Z is selected from hydrogen and (1-3C)alkyl (preferably hydrogen); and

the anilino group at the 4-position on the quinazoline ring is selected from 3-chloro-4-fluoroanilino, 3-bromo-2-fluoroanilino and particularly 3-chloro-2-fluoroanilino;

or a pharmaceutically acceptable salt thereof.

In another embodiment of the invention there is provided a quinazoline derivative of the Formula I of the Formula IE:

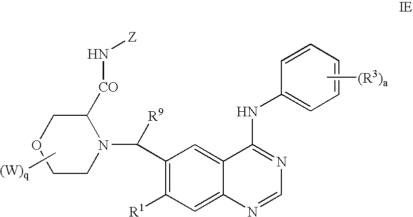

wherein R¹, R³, Z, W and q have any of the values defined herein in relation to Formula I;

R⁹ is selected from hydrogen and (1-3C)alkyl (for example R⁹ is hydrogen or methyl, preferably R⁹ is hydrogen); and

a is 1, 2 or 3 (preferably 1 or 2) and each R³, which may be the same or different is selected from fluoro, chloro, bromo and ethynyl;

or a pharmaceutically acceptable salt thereof.

In the quinazoline derivative of Formula IE a particular value for R¹ is (1-4C)alkoxy, hydroxy-(2-4C)alkoxy and (1-3C)alkoxy-(2-4C)alkoxy. More particularly R¹ is (1-4C)alkoxy such as methoxy, ethoxy or isopropyloxy.

In the quinazoline derivative of Formula IE a particular value for q is 0 or 1 and W is selected from halogeno, (1-4C)alkyl, hydroxy-(1-4C)alkyl and (1-4C)alkoxy-(1-4C)alkyl. Particularly W is selected from (1-3C)alkyl, hydroxy-(1-3C)alkyl and (1-3C)alkoxy-(1-3C)alkyl.

A particular value for Z in Formula IE is a group selected from hydrogen and (1-3C)alkyl. Preferably however, Z is hydrogen.

In this embodiment a particular anilino group at the 4-position on the quinazoline ring in Formula IE is selected from 3-chloro-4-fluoroanilino, 3-bromo-2-fluoroanilino, 3-chloro-2-fluoroanilino, 3-bromoanilino and 3-ethynylanilino. Still more particularly the anilino group is 3-bromo-2-fluoroanilino or, preferably, 3-chloro-2-fluoroanilino.

Accordingly a particular quinazoline derivative is of the Formula IE as hereinbefore defined wherein:

R¹ is (1-4C)alkoxy;

R⁹ is hydrogen or methyl (preferably hydrogen);

q is 0, 1 or 2 (preferably 0 or 1) and W has any of the values defined hereinbefore for W in relation to the quinazoline derivative of Formula I (particularly W is selected from (1-3C)alkyl, (1-3C)alkoxy, hydroxy-(1-3C)alkyl and (1-3C)alkoxy-(1-3C)alkyl);

Z is selected from hydrogen and (1-3C)alkyl (preferably hydrogen); and

the anilino group at the 4-position on the quinazoline ring is selected from 3-chloro-4-fluoroanilino, 3-bromo-2-fluoroanilino and particularly 3-chloro-2-fluoroanilino;

or a pharmaceutically acceptable salt thereof.

In another embodiment of the invention there is provided a quinazoline derivative of the Formula I of the Formula IF:

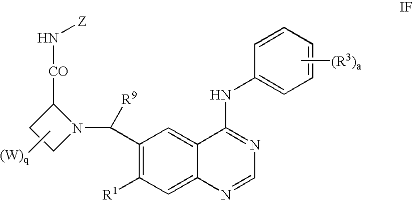

wherein R¹, R³, W, q and Z have any of the values defined herein in relation to Formula I;

R⁹ is selected from hydrogen and (1-3C)alkyl (for example R⁹ is hydrogen or methyl, preferably R⁹ is hydrogen); and

a is 1, 2 or 3 (preferably 1 or 2) and each R³, which may be the same or different is selected from fluoro, chloro, bromo and ethynyl;

or a pharmaceutically acceptable salt thereof.

In the quinazoline derivative of Formula IF a particular value for R¹ is (1-4C)alkoxy, hydroxy-(2-4C)alkoxy and (1-3C)alkoxy-(2-4C)alkoxy. More particularly R¹ is (1-4C)alkoxy such as methoxy, ethoxy or isopropyloxy.

In the quinazoline derivative of Formula IF a particular value for q is 0 or 1 and W is selected from halogeno, hydroxy, (1-4C)alkyl, hydroxy-(1-4C)alkyl and (1-4C)alkoxy-(1-4C)alkyl. Particularly W is selected from hydroxy, (1-3C)alkyl, hydroxy-(1-3C)alkyl and (1-3C)alkoxy-(1-3C)alkyl. Preferably q is 0 or 1 and W when present is at the 3-position on the azetidin-1-yl ring in Formula IF.

A particular value for Z in Formula IF is a group selected from hydrogen and (1-3C)alkyl. Preferably however, Z is hydrogen.

In this embodiment a particular anilino group at the 4-position on the quinazoline ring in Formula IF is selected from 3-chloro-4-fluoroanilino, 3-bromo-2-fluoroanilino, 3-chloro-2-fluoroanilino, 3-bromoanilino and 3-ethynylanilino. Still more particularly the anilino group is 3-bromo-2-fluoroanilino or, preferably, 3-chloro-2-fluoroanilino.

Accordingly a particular quinazoline derivative is of the Formula IF as hereinbefore defined wherein:

R¹ is (1-4C)alkoxy;

R⁹ is hydrogen or methyl (preferably hydrogen);

q is 0, 1 or 2 (preferably 0 or 1), W is at the 3-position on the azetidin-1-yl ring and has any of the values defined hereinbefore for W in relation to the quinazoline derivative of Formula I (particularly W is selected from hydroxy, (1-3C)alkyl, (1-3C)alkoxy, hydroxy-(1-3C)alkyl and (1-3C)alkoxy-(1-3C)alkyl, for example W is hydroxy, methoxy or ethoxy);

Z is selected from hydrogen and (1-3C)alkyl (preferably hydrogen); and

the anilino group at the 4-position on the quinazoline ring is selected from 3-chloro-4-fluoroanilino, 3-bromo-2-fluoroanilino and particularly 3-chloro-2-fluoroanilino;

or a pharmaceutically acceptable salt thereof.

A particularly preferred sub-group of quinazoline derivatives of Formula I are compounds of the Formula IG

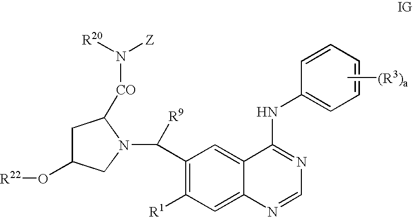

wherein R¹, R³, R²⁰, a and Z have any of the values defined hereinbefore in relation to Formula I;

R⁹ is hydrogen or methyl (preferably hydrogen); and

R²² is selected from hydrogen, (1-6C)alkyl, (2-6C)alkenyl, (2-6C)alkynyl, (2-6C)alkanoyl and a group R¹⁰ wherein R¹⁰ is as defined above in relation to Formula I.

Particular examples of R²² include hydrogen, (1-6C)alkyl such as methyl, ethyl, propyl, iso-propyl, n-butyl, halogeno-(1-6C)alkyl, hydroxy-(2-6C)alkyl or (1-6C)alkoxy-(2-6C)alkyl;

or a pharmaceutically acceptable salt thereof.

Further examples of R²² include hydrogen, (1-4C)alkyl, halogeno-(1-4C)alkyl, hydroxy-(2-4C)alkyl or (1-3C)alkoxy-(2-4C)alkyl.

More particularly, R²² may be hydrogen or (1-6Calkyl), still more particularly R²² is (1-4C)alkyl such as methyl or ethyl.

In this embodiment of the invention in Formula IG, Z and R²⁰ are suitably hydrogen.

Accordingly a particular quinazoline derivative is of the Formula IG as hereinbefore defined wherein:

R¹ is (1-4C)alkoxy;

R⁹ is hydrogen or methyl (preferably methyl);

R²⁰ is hydrogen;

Z is selected from hydrogen and (1-3C)alkyl (preferably hydrogen);

the anilino group at the 4-position on the quinazoline ring is selected from 3-chloro-4-fluoroanilino, 3-bromo-2-fluoroanilino and particularly 3-chloro-2-fluoroanilino; and

R²² has any of the values defined herein, particularly hydrogen or (1-3C)alkyl;

or a pharmaceutically acceptable salt thereof.

It is to be understood that, insofar as certain of the compounds of Formula I defined above may exist in optically active or racemic forms by virtue of one or more asymmetrically substituted carbon and/or sulfur atoms, and accordingly may exist in, and be isolated as enantiomerically pure, a mixture of diastereoisomers or as a racemate. The present invention includes in its definition any racemic, optically-active, enantiomerically pure, mixture of diastereoisomers, stereoisomeric form of the compound of Formula (I), or mixtures thereof, which possesses the above-mentioned activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

The invention relates to all tautomeric forms of the compounds of the Formula I that possess antiproliferative activity.

It is also to be understood that certain compounds of the Formula I may exist in solvated as well as unsolvated forms such as, for example, hydrated forms. It is to be understood that the invention encompasses all such solvated forms which possess antiproliferative activity.

It is also to be understood that certain compounds of the Formula I may exhibit polymorphism, and that the invention encompasses all such forms which possess antiproliferative activity.

A suitable pharmaceutically-acceptable salt of a compound of the Formula I is, for example, an acid-addition salt of a compound of the Formula I, for example an acid-addition salt with an inorganic or organic acid such as hydrochloric, hydrobromic, sulfuric, trifluoroacetic, citric or maleic acid; or, for example, a salt of a compound of the Formula I which is sufficiently acidic, for example an alkali or alkaline earth metal salt such as a calcium or magnesium salt, or an ammonium salt, or a salt with an organic base such as methylamine, dimethylamine, trimethylamine, piperidine, morpholine or tris-(2-hydroxyethyl)amine.

A preferred compound of the invention is, for example, a quinazoline derivative of the Formula I selected from the compounds illustrated in Tables I and II:

TABLE I

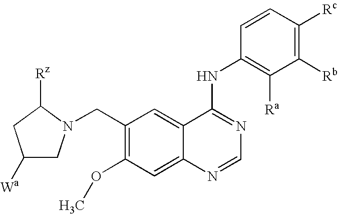

| Compound | | | | | |
|---|---|---|---|---|---|
| No. | R^{z} | R^{a} | R^{b} | R^{c} | W^{a} |
| 1 | 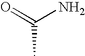 | F | Cl | H | H |
| 2 | 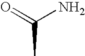 | F | Cl | H | H |
| 3 | 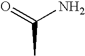 | H | Cl | F | H |
| 4 | 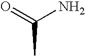 | F | Cl | H | HO [symbol] |
| 5 | 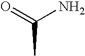 | F | Cl | H | HO [symbol] |
| 6 | 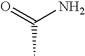 | F | Cl | H | HO [symbol] |
| 7 | 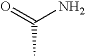 | F | Cl | H | HO [symbol] |
| 8 | 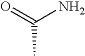 | H | Cl | F | H |
| 9 | 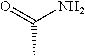 | H | Cl | F | HO [symbol] |
| 13 | 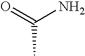 | F | Cl | H | MeO [symbol] |
| 14 | 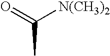 | F | Cl | H | H |

TABLE II

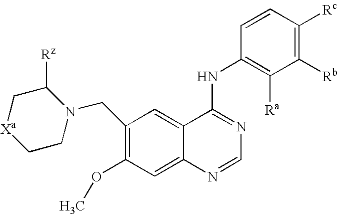

| Compound | | | | | |
|---|---|---|---|---|---|
| No. | R^{z} | R^{a} | R^{b} | R^{c} | X^{a} |
| 10 | 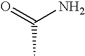 | F | Cl | H | CH₂ |
| 11 | 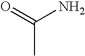 | F | Cl | H | O |
| 12 | 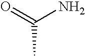 | H | Cl | F | CH₂ |
| 15 | 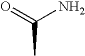 | F | Cl | H | CH₂ |

A particular compound of the invention is, for example, a quinazoline derivative of the Formula I selected from:
1-({4-[(3-chloro-2-fluorophenyl)amino]-7-methoxyquinazolin-6-yl}methyl)-L-prolinamide;
1-({4-[(3-chloro-2-fluorophenyl)amino]-7-methoxyquinazolin-6-yl}methyl)-D-prolinamide;
(4R)-1-({4-[(3-chloro-2-fluorophenyl)amino]-7-methoxyquinazolin-6-yl}methyl)-4-hydroxy-L-prolinamide;
(4S)-1-({4-[(3-chloro-2-fluorophenyl)amino]-7-methoxyquinazolin-6-yl}methyl)-4-hydroxy-L-prolinamide;
(4S)-1-({4-[(3-chloro-2-fluorophenyl)amino]-7-methoxyquinazolin-6-yl}methyl)-4-hydroxy-D-prolinamide;
(4R)-1-({4-[(3-chloro-2-fluorophenyl)amino]-7-methoxyquinazolin-6-yl}methyl)-4-hydroxy-D-prolinamide;
1-({4-[(3-chloro-4-fluorophenyl)amino]-7-methoxyquinazolin-6-yl}methyl)-L-prolinamide;
1-({4-[(3-chloro-4-fluorophenyl)amino]-7-methoxyquinazolin-6-yl}methyl)-D-prolinamide;
(4R)-1-({4-[(3-chloro-4-fluorophenyl)amino]-7-methoxyquinazolin-6-yl}methyl)-4-hydroxy-D-prolinamide;
(4R)-1-({4-[(3-Chloro-2-fluorophenyl)amino]-7-methoxyquinazolin-6-yl}methyl)-4-hydroperoxy-D-prolinamide;
1-({4-[(3-chloro-2-fluorophenyl)amino]-7-methoxyquinazolin-6-yl}methyl)-D-proline; and
1-({4-[(3-chloro-2-fluorophenyl)amino]-7-methoxyquinazolin-6-yl}methyl)-N,N-dimethyl-L-prolinamide;
   or a pharmaceutically acceptable salt thereof.

Another particular compound of the invention is, for example, a quinazoline derivative of the Formula I selected from:
(4R)-3-({4-[(3-chloro-2-fluorophenyl)amino]-7-methoxyquinazolin-6-yl}methyl)-1,3-thiazolidine-4-carboxamide;
(3S)-1-({4-[(3-chloro-2-fluorophenyl)amino]-7-methoxyquinazolin-6-yl}methyl)-3-hydroxy-L-prolinamide
(4R)-1-({4-[(3-chloro-2-fluorophenyl)amino]-7-methoxyquinazolin-6-yl}methyl)-4-ethoxy-D-prolinamide;
1-({4-[(3-chloro-2-fluorophenyl)amino]-7-methoxyquinazolin-6-yl}methyl)-2-methylprolinamide; and
(1S,5R)-3-({4-[(3-Chloro-2-fluorophenyl)amino]-7-methoxyquinazolin-6-yl}methyl)-3-azabicyclo[3.1.0]hexane-2-carboxamide
   or a pharmaceutically acceptable salt thereof.

A further aspect the present invention provides a process for preparing a quinazoline derivative of Formula I or a pharmaceutically-acceptable salt thereof. It will be appreciated that during certain of the following processes certain substituents may require protection to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Thus, if reactants include, for example, groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon. Resins may also be used as a protecting group.

The protecting groups may be removed at any convenient stage in the synthesis using conventional techniques well known in the chemical art.

A quinazoline derivative of the Formula I, or a pharmaceutically-acceptable salt thereof, may be prepared by any process known to be applicable to the preparation of chemically-related compounds. Such processes, when used to prepare a quinazoline derivative of the Formula I, or a pharmaceutically-acceptable salt thereof, are provided as a further feature of the invention and are illustrated by the following representative examples. Necessary starting materials may be obtained by standard procedures of organic chemistry (see, for example, Advanced Organic Chemistry (Wiley-Interscience), Jerry March). The preparation of such starting materials is described within the accompanying non-limiting Examples. Alternatively, necessary starting materials are obtainable by analogous procedures to those illustrated which are within the ordinary skill of an organic chemist. Information on the preparation of necessary starting materials or related compounds (which may be adapted to form necessary starting materials) may also be found in the following Patent and Application Publications, the contents of the relevant process sections of which are hereby incorporated herein by reference: WO94/27965, WO 95/03283, WO 96/33977, WO 96/33978, WO 96/33979, WO 96/33980, WO 96/33981, WO 97/30034, WO 97/38994, WO01/66099, U.S. Pat. No. 5,252,586, EP 520 722, EP 566 226, EP 602 851 and EP 635 507.

The present invention also provides that quinazoline derivatives of the Formula I, or pharmaceutically acceptable salts thereof, can be prepared by a process as follows (wherein the variables are as defined above unless otherwise stated):

The present invention also provides methods for preparing quinazoline derivatives of the Formula I, or pharmaceutically acceptable salts thereof, as outlined below.

It will be appreciated that during certain of the following processes certain substituents may require protection to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Thus, if reactants include, for example, groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon. Resins may also be used as a protecting group.

The protecting groups may be removed at any convenient stage in the synthesis using conventional techniques well known in the chemical art.

### Process (a)

the reaction of a compound of formula (II):

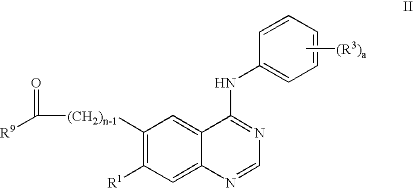

wherein n, a, R¹, R³ and R⁹ is defined in relation to Formula I, except that any functional group is protected if necessary, with a compound of formula (III):

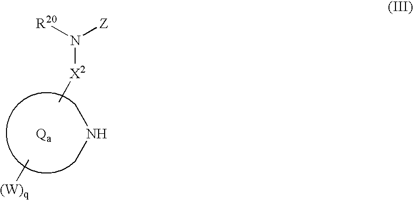

wherein X², W, Z, R²⁰ b and Q^{a} are as defined in relation to Formula I, except that any functional group is protected if necessary; or

### Process (b):

the reaction of a compound of formula (XX):

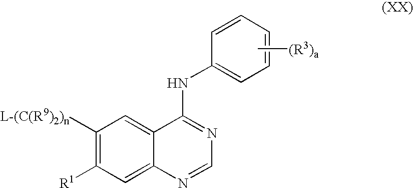

wherein R¹, R³, R⁹, n and a are as defined in relation to Formula I except that any functional group is protected if necessary, and L is a leaving group, such as mesylate, tosylate or halogeno, with a compound of formula (III) as defined above in relation to Process (a); or

### Process (c)

for the preparation of quinazoline derivatives of the Formula I wherein X² is C(O), the coupling, conveniently in the presence of a suitable base, of a quinazoline of the formula (XXI) or a reactive derivative thereof:

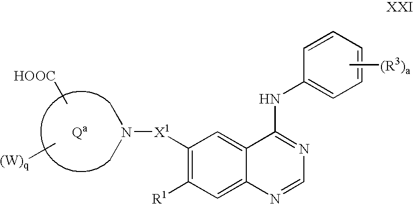

wherein R¹, R³, W, a, q, X¹ and Q^{a} have any of the meanings defined hereinbefore except that any functional group is protected if necessary, with a compound of the formula XXII, or a salt thereof:

HN(R²⁰)Z XXII

wherein R²⁰ and Z have any of the meanings defined hereinbefore except that any functional group is protected if necessary;

### Process (d)

the reductive amination of the corresponding quinazoline derivative of the Formula I which contains an NH group with an appropriate aldehyde; or

### Process (e)

for the production of those quinazoline derivatives of the Formula I wherein R¹ is hydroxy, the cleavage of a quinazoline derivative of the Formula I wherein R¹ is a (1-6C)alkoxy group; or

### Process (f)

for the production of those quinazoline derivatives of the Formula I wherein R¹ is linked to the quinazoline ring by an oxygen atom, by coupling a compound of the formula (XXIII):

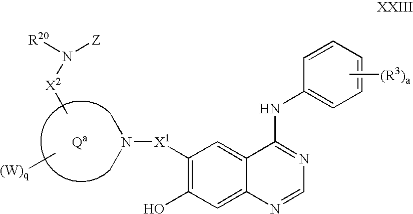

wherein R³, R²⁰, Z, W, a, q, X¹, X² and Q^{a} have any of the meanings defined hereinbefore except that any functional group is protected if necessary, with a compound of the formula R^{1′} OH wherein R^{1′} is one of the oxygen linked groups as hereinbefore defined for R¹ (for example Q¹-O—), except that any functional group is protected if necessary;

and thereafter, if necessary (in any order):
(i) converting a quinazoline derivative of the Formula I into another quinazoline derivative of the Formula I;
(ii) removing any protecting group that is present by conventional means; and
(iii) forming a pharmaceutically acceptable salt.
Specific conditions for the above reactions are as follows:

### Reaction Conditions for Process (a)

The reaction is suitably performed under reductive amination conditions as described below in relation to Process (d). Suitably, the reaction is carried out in the presence of a reducing agent, in particular a Lewis acid such as a boron compound, or hydrogen. A particular example is sodium triacteoxyborohydride, sodium cyanoborohydride, sodium borohydride or polymer supported borohydride. The reaction is suitably effected in an organic solvent such as tetrahydrofuran (THF), dichloromethane, 1,2-dichloroethane, or an allyl alcohol such as methanol or ethanol. Moderate temperatures for example of from 0-60° C., and conveniently at ambient temperature, are suitably employed. The reaction may also be preformed in the presence of a drying or dehydrating agent, typically magnesium sulfate or molecular sieves as this which helps drive the forward reaction.

If desired, optically active or resolved forms of compounds of formula (III) may be employed, to produce optically active compounds of Formula I.

Process (a) is particularly suitable for the preparation of quinazoline derivatives of the Formula I wherein n is 1.

### Preparation of Starting Materials for Process (a)

Compounds of formula (II) are suitably prepared by oxidising a compound of formula (IV)

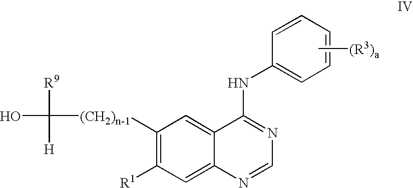

wherein R⁹, R¹, R³, n and a are as defined in relation to Formula I, but wherein any functional groups are protected as necessary. Oxidation is suitably effected using an oxidising agent such as manganese oxide, Tetra-n-propylammonium perruthenate (TPAP)/N-methylmorpholine N-oxide or by employing Swern conditions (e.g oxidation promoted by oxalyl chloride activation of dimethyl sulfoxide (DMSO) upon the addition of a base such as tri-ethylamine). In an organic solvent such as methylene chloride, methanol, dioxane, dichloromethane, 1,2 dichloroethane or THF. Again moderate temperatures for example of from 0-50° C. and conveniently ambient temperatures are suitably employed. The reaction is continued for a sufficient period of time to allow oxidation to take place. If necessary, the product can be separated using column chromatography, for example on a silica column.

Alternatively, compounds of formula (II) where n is 1 and R⁹ is hydrogen, are prepared by for example, hydroformylation of a compound of formula (VII) as defined below. In that case, the reaction is suitably effected by reacting a compound of formula (VII) with carbon monoxide and a reducing agent such as trioctyl silane or triethyl silane, in the presence of a palladium catalyst such as palladium acetate, which is suitably combined with a strong electron donor, such as diphenylphosphinopropane and a base such as triethylamine. The reaction is suitably carried out in the presence of an inert solvent or diluent, for example N,N-dimethylformamide. The reaction is suitably carried out at elevated temperature, for example from 40 to 100° C., such as approximately 70° C.

Compounds of formula (II) where n is 1 and R⁹ is methyl, can be prepared by for example, reaction of a compound of formula (VII) as defined below with a (1-6C)alkyl vinyl ether, such as n-butyl vinyl ether, in the presence of a palladium catalyst such as palladium acetate, which is suitably combined with a strong electron donor, such as diphenylphosphinopropane and a base such as triethylamine. Following the reaction the resulting ether is treated with an acid to give a compound of formula II. The reaction is suitably carried out in the presence of an inert solvent or diluent and under analogous conditions to the hydroformylation reaction described above.

Compounds of formula (IV) where R⁹ is hydrogen are suitably prepared by reduction of a compound of formula (V)

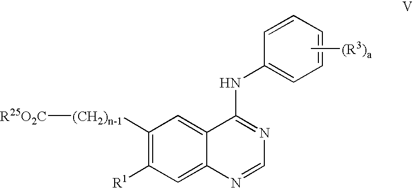

wherein R¹, R³ n and a are as defined in relation to Formula I, except that any functional group is protected if necessary, and R²⁵ is an acid protecting group, such as (1-6C)alkyl. The reduction reaction is suitably carried out using a reducing agent such as lithium aluminium hydride (LiAlH₄), diisobutylaluminum hydride (DIBAL-H), sodium borohydride (NaBH₄) or BH3.S(CH₃)₂. A particular reducing agent which may be used in this process is Red-Al, a compound of formula (VI)

([CH₂OCH₂OCH₂)₂AlH₂]Na (VI)

which is obtainable as a solution, for example of 65-70% w/w in organic solvents such as hexane, or toluene. The reaction is suitably effected in an organic solvent such as THF, at low or moderate temperatures, for example of from −100 to 60° C. At the end of the reaction with Red-AL, the reaction may be quenched, for example sodium hydrogen tartrate in water.

Compounds of formula (V) where n is 1 may be prepared by hydrocarboxylation of a compound of formula (VII)

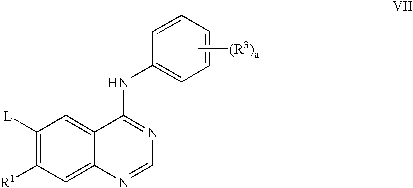

wherein R¹, R³ and a are as defined in relation to Formula I, except that any functional group is protected if necessary, and L represents a leaving group. In particular, such a reaction is effected by reacting the compound of formula (VII) with carbon monoxide and an alcohol of formula R²⁵OH, where R²⁵ is as defined above in relation to formula (V), in the presence of a palladium catalyst such as palladium acetate, which is suitably combined with a strong electron donor, such as diphenylphosphinopropane and a base such as triethylamine. The reaction is suitably carried out in the presence of an inert solvent or diluent, for example N,N-dimethylformamide. The reaction is suitably carried out at elevated temperature, for example from 40 to 100° C., such as approximately 70° C.

Particular examples of leaving groups L in formula V include trifluoromethanesulfonyloxy or halogeno such as chloro, bromo or iodo.

Compounds of formula (VII) are suitably prepared by reacting a compound of formula (VIII)

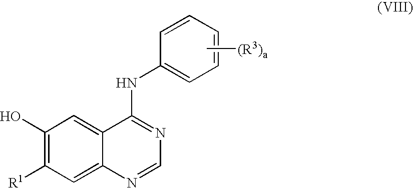

wherein R¹, R³ and a are as defined in relation to Formula I except that any functional group is protected if necessary, with a halogenating agent or a compound of formula (IX)

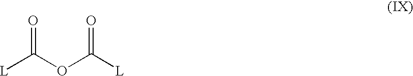

where L is a leaving group other than halogeno. The reaction is suitably effected in an inert organic solvent such as methylene chloride, THF or 1,2-dichloroethane in the presence of a base such as pyridine, triethylamine, diisopropylethylamine or 4-dimethylaminopyridine (4-DMAP). Low temperatures, for example of from −20 to 20° C., and preferably about 0° C. are suitably employed.

A particular example of a compound of formula (IX) is trifluoromethanesulfonic acid anhydride or triflic anhydride.

Compounds of formula (VIII) are known or can be prepared using conventional techniques or analogous processes to those described in the prior art. In particular those patents and applications listed hereinbefore, such as WO96/15118, WO 01/66099 and EP 566 226. For example, the compounds of formula (VIII) may be prepared in accordance with Reaction Scheme 1:

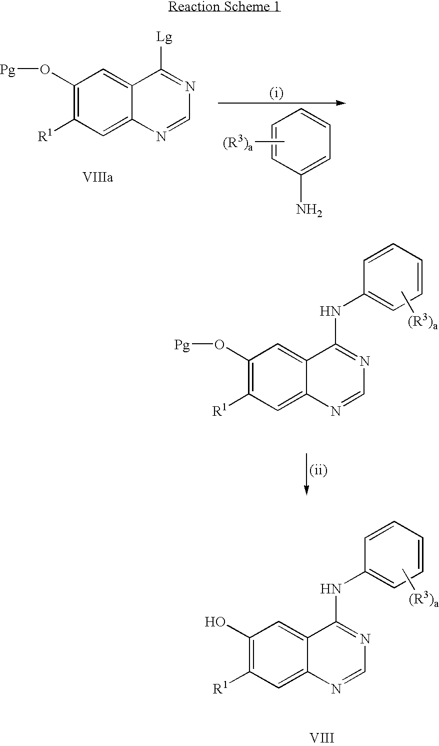

wherein R¹, X¹, G¹ and G² are as hereinbefore defined, Pg is a hydroxy protecting group, and Lg is a leaving group as defined herein for L.

### Notes for Reaction Scheme 1

Step (i): Reaction suitably in an inert protic solvent (such as an alkanol for example iso-propanol), an aprotic solvent (such as dioxane) or a dipolar aprotic solvent (such as N,N-dimethylacetamide) in the presence of an acid, for example hydrogen chloride gas in diethyl ether or dioxane, or hydrochloric acid, under analogous conditions to those described above under Process (a).

Alternatively the reaction may be carried out in one of the above inert solvents conveniently in the presence of a base, for example potassium carbonate. The above reactions are conveniently carried out at a temperature in the range, for example, 0 to 150° C., suitably at or near the reflux temperature of the reaction solvent.

Step (ii): Cleavage of Pg may be performed under standard conditions for such reactions. For example when Pg is an alkanoyl group such as acetyl, it may be cleaved by heating in the presence of a methanolic ammonia solution.

Compounds of formula VIIIa are known or can be prepared using known processes for the preparation of analogous compounds. If not commercially available, compounds of the formula (VIII) may be prepared by procedures which are selected from standard chemical techniques, techniques which are analogous to the synthesis of known, structurally similar compounds, or techniques which are analogous to the procedures described in the Examples. For example, standard chemical techniques are as described in Houben Weyl. By way of example the compound of the formula VIII in which R¹ is methoxy, Lg is chloro and Pg is acetyl may be prepared using the process illustrated in Reaction Scheme 2:

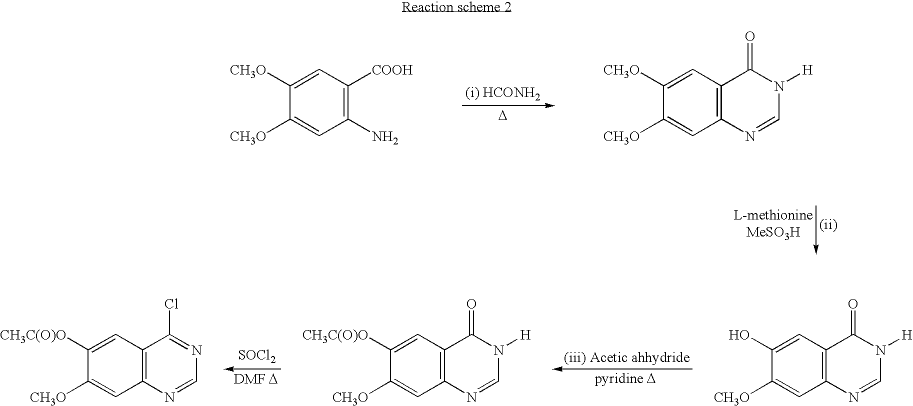

Reaction Scheme 2 may be generalised by the skilled man to apply to compounds within the present specification which are not specifically illustrated (for example to introduce a substituent other than methoxy at the 7-position in the quinazoline ring).

Compounds of formula (V) wherein n is 2 can be prepared by reacting a compound of formula (VII) where L is a leaving group such as OTf, where Tf is a trifluoromethylsulfonyl group, with a compound of formula (X):

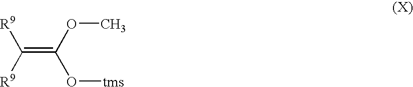

where R⁹ is as defined above and tms is a trimethylsilyl group, in the presence of a palladium catalyst using a method analogous to that described in J. Organic Chemistry 1991, 56(1) p 261.

Alternatively, a compound of formula (IV) where n is 2 can be prepared by reduction of a compound of formula (XI)

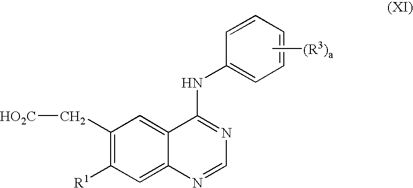

wherein R¹, R³ and a are as defined in relation to Formula I, provided that any functional group is protected if necessary. Suitable reduction conditions will be similar to those described above for the reduction of the compound of formula (V).

Compounds of formula (XI) can be prepared by subjecting a compound of formula (XII)

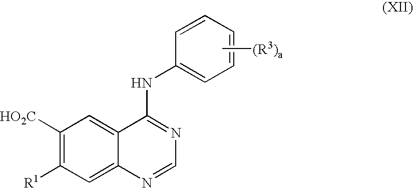

wherein R¹, R³ and a are as defined in relation to Formula I, and provided any functional groups are protected as necessary, to an Arndt-Eistert homologation, as described for example by H. Meier et al., Chem Int Ed. Engl., 1975, 14, 32. This reaction comprises:
i) acid chloride formation (for example using (COCl)₂/DMF/CH₂Cl₂ at 0° C.-room temperature;
ii) diazoketone formation (for example using diazomethane or TMS diazomethane/diethyl ether/tetrahydrofuran at 0° C.-room temperature; and
iii) a Wolff rearrangement using H₂O, and heat in the presence of an Ag₂O catalyst.

Compounds of formula (XII) are suitably prepared by hydrolysis of a compound of formula (V) where n is 1. Hydrolysis may suitably be carried out using an alkyl alcohol such as methanol, in the presence of a base such as sodium or lithium hydroxide in an organic solvent such as THF. Temperatures ranging from ambient temperatures to the reflux temperature of the solvent are suitably employed.

Compounds of formula (III) are known or can be prepared using conventional techniques or analogous processes to those described in the prior art. For example when X² is C(O) by amide formation from the corresponding carboxylic acid and if required functional group modification to provide alternative amides and/or W groups. Such transformations well known and are illustrated in the examples herein.

### Reaction Conditions for Process (b)

Suitable reaction conditions would be apparent to a skilled chemist. Generally the reaction would be effected in an inert organic solvent such as dichloromethane, dichloroethane, DMA, DMF etc in the presence of a base such as DIPEA, triethylamine, potassium carbonate, caesium carbonate etc. Temperatures in the range of from 0° C. to 200° C. are suitably employed, and conveniently at or near boiling point of solvent.

### Preparation of Starting Materials for Process (b)

Compounds of formula (XX) can be prepared by conventional methods, for example by reacting a compound of formula (IV) as described above in relation to Process (a) with a halogenating agent or a compound of formula (IX) as defined hereinbefore in relation to the preparation of starting materials for Process (a). The reaction is suitably effected in an inert organic solvent such as methylene chloride, THF or 1,2-dichloroethane in the presence of a base such as pyridine, triethylamine, diisopropylethylamine or 4-DMAP. Low temperatures, for example of from −20 to 20° C., and preferably about 0° C. are suitably employed.

### Reaction Conditions for Process (c)

The coupling reaction of the acid of formula XX is conveniently carried out in the presence of a suitable coupling agent, such as a carbodiimide, or 1-hydroxybenztriazole or a uronium coupling agent. Suitable uronium coupling agents include, for example O-(7-azabenzotriazol-1-yl)-N,N,N′,N′-tetramethyluronium hexafluoro-phosphate (HATU) or O-(1H-Benzotriazol-1-yl)-N,N,N′,N′-tetramethyl uronium tetrafluoroborate (TBTU). A suitable carbodiimide includes dicyclohexylcarbodiimide or 1-ethyl-3-(3-dimethylaminopropyl)carbodiimide. The reaction is conveniently carried out in the presence of a catalyst such as dimethylaminopyridine or 4-pyrrolidinopyridine.

The coupling reaction is conveniently carried out in the presence of a suitable base. A suitable base is, for example, an organic amine base such as, for example, pyridine, 2,6-lutidine, collidine, 4-dimethylaminopyridine, triethylamine, di-isopropylethylamine, N-methylmorpholine or diazabicyclo[5.4.0]undec-7-ene, or, for example, an alkali or alkaline earth metal carbonate, for example sodium carbonate, potassium carbonate, cesium carbonate or calcium carbonate.

The reaction is conveniently carried out in the presence of a suitable inert solvent or diluent, for example an ester such as or ethyl acetate, a halogenated solvent such as methylene chloride, chloroform or carbon tetrachloride, an ether such as tetrahydrofuran or 1,4-dioxan, an aromatic solvent such as toluene, or a dipolar aprotic solvent such as N,N-dimethylformamide, N,N-dimethylacetamide, N-methylpyrrolidin-2-one or dimethylsulfoxide. The reaction is conveniently carried out at a temperature in the range, for example, from −20 to 120° C., conveniently at or near ambient temperature.

By the term “reactive derivative” of the acid of the formula XX is meant a carboxylic acid derivative that will react with the amine formula XXII to give the corresponding amide. A suitable reactive derivative of a carboxylic acid of the formula XXI is, for example, an acyl halide, for example an acyl chloride formed by the reaction of the acid and an inorganic acid chloride, for example thionyl chloride; a mixed anhydride, for example an anhydride formed by the reaction of the acid and a chloroformate such as an alkyl chloroformate, for example ethyl chloroformate or isobutyl chloroformate; an active ester, for example an ester formed by the reaction of the acid and a phenol such as pentafluorophenol, or N-hydroxybenzotriazole; or an acyl azide, for example an azide formed by the reaction of the acid and azide such as diphenylphosphoryl azide; an acyl cyanide, for example a cyanide formed by the reaction of an acid and a cyanide such as diethylphosphoryl cyanide. The reaction of such reactive derivatives of carboxylic acid with amines (such as a compound of the formula XXII) is well known in the art, for example they may be reacted in the presence of a base, such as those described above, and in a suitable solvent, such as those described above. The reaction may conveniently be performed at a temperature as described above.

### Preparation of Starting Materials for Process (c)

Compounds of the formula (XXI) may be prepared by for example reacting a compound of the formula (II) with a compound of the formula (IIIa):

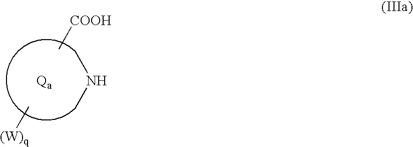

wherein W, Q^{a} and q are as defined in relation to Formula I, and provided any functional groups are protected as necessary. The reaction is suitably carried out under analogous conditions to those used in Process (a) herein.

Compounds of the formulae (IIIa) and (XXII) are known or can be prepared using conventional techniques or analogous processes to those described in the prior art.

### Reaction Conditions for Process (d)

Suitable reductive amination conditions are well known in the art, for example, as described in relation to Process (a) herein. A quinazoline derivative of Formula I, which contains an NH group (for example when Q^{a} is piperazin-1-yl) is reacted with an appropriate aldehyde to give an optionally substituted ring N(alkyl) group. Appropriate aldehydes will be apparent, for example for the production of those quinazoline derivatives of the Formula I wherein Q^{a} contains a ring N-methyl group, the corresponding compound containing a ring N—H group may be reacted with formaldehyde in the presence of a suitable reducing agent. Similarly to give an optionally substituted ring N(alkyl) group a suitable aldehyde is the corresponding optionally substituted (2-6C)alkanolaldehyde (for example acetaldehyde, propionaldehyde or (1-4C)alkoxyacetaldehyde such as methoxyacetaldehyde).

A suitable reducing agent is, for example, a hydride reducing agent, for example formic acid, an alkali metal aluminium hydride such as lithium aluminium hydride, or, suitably, an alkali metal borohydride such as sodium borohydride, sodium cyanoborohydride, sodium triethylborohydride, sodium trimethoxyborohydride and sodium triacetoxyborohydride. The reaction is conveniently performed in a suitable inert solvent or diluent, for example tetrahydrofuran and diethyl ether for the more powerful reducing agents such as lithium aluminium hydride, and, for example, methylene chloride or a protic solvent such as methanol and ethanol for the less powerful reducing agents such as sodium triacetoxyborohydride and sodium cyanoborohydride. The reaction is suitably performed under acidic conditions in the presence of a suitable acid such as hydrogen chloride or acetic acid, a buffer may also be used to maintain pH at the desired level during the reaction. When the reducing agent is formic acid the reaction is conveniently carried out using an aqueous solution of the formic acid. The reaction is performed at a temperature in the range, for example, −10 to 100° C., such as 0 to 50° C., conveniently, at or near ambient temperature.

Reaction conditions for Process (e)

The cleavage reaction may conveniently be carried out by any of the many procedures known for such a transformation. A particularly suitable cleavage reaction is the treatment of a quinazoline derivative of the Formula I wherein R¹ is a (1-6C)alkoxy group with pyridinium hydrochloride, or an alkali metal halide such as lithium iodide in the presence of 2,4,6-collidine (2,4,6-trimethylpyridine). The reaction may be carried out in the presence of a suitable inert solvent or diluent as defined hereinbefore. The reaction is suitably carried out at a temperature in the range, for example, 10 to 170° C., preferably at elevated temperature for example 120 to 170° C., for example approximately 130° C.

### Reaction Conditions for Process (f)

The coupling reaction is conveniently carried out under Mitsunobu conditions. Suitable Mitsunobu conditions are well known and include, for example, reaction in the presence of a suitable tertiary phosphine and a di-alkylazodicarboxylate in an organic solvent such as THF, or suitably dichloromethane and in the temperature range 0° C. to 100° C., for example 0° C. to 60° C., but suitably at or near ambient temperature. A suitable tertiary phosphine includes for example tri-n-butylphosphine or particularly tri-phenylphosphine. A suitable di-alkylazodicarboxylate includes, for example, diethyl azodicarboxylate (DEAD) or suitably di-tert-butyl azodicarboxylate (DTAD). Details of Mitsunobu reactions are contained in Tet. Letts., 31, 699, (1990); The Mitsunobu Reaction, D. L. Hughes, Organic Reactions, 1992, Vol. 42, 335-656 and Progress in the Mitsunobu Reaction, D. L. Hughes, Organic Preparations and Procedures International, 1996, Vol. 28, 127-164.

The compound of formula XXIII used as starting material may be prepared by, for example, the cleavage of a quinazoline derivative of the Formula I, wherein R¹ is, for example, methoxy using Process (e) described hereinbefore.

The quinazoline derivative of the Formula I may be obtained from the above processes in the form of the free base or alternatively it may be obtained in the form of a salt, an acid addition salt. When it is desired to obtain the free base from a salt of the compound of Formula I, the salt may be treated with a suitable base, for example, an alkali or alkaline earth metal carbonate or hydroxide, for example sodium carbonate, potassium carbonate, calcium carbonate, sodium hydroxide or potassium hydroxide, or by treatment with ammonia for example using a methanolic ammonia solution such as 7N ammonia in methanol.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group.

When a pharmaceutically-acceptable salt of a quinazoline derivative of the Formula I is required, for example an acid-addition salt, it may be obtained by, for example, reaction of said quinazoline derivative with a suitable acid using a conventional procedure.

As mentioned hereinbefore some of the compounds according to the present invention may contain one of more chiral centers and may therefore exist as stereoisomers. Stereoisomers may be separated using conventional techniques, e.g. chromatography or fractional crystallisation. The enantiomers may be isolated by separation of a racemate for example by fractional crystallisation, resolution or HPLC. The diastereoisomers may be isolated by separation by virtue of the different physical properties of the diastereoisomers, for example, by fractional crystallisation, HPLC or flash chromatography. Alternatively particular stereoisomers may be made by chiral synthesis from chiral starting materials under conditions which will not cause racemisation or epimerisation, or by derivatisation, with a chiral reagent. When a specific stereoisomer is isolated it is suitably isolated substantially free for other stereoisomers, for example containing less than 20%, particularly less than 10% and more particularly less than 5% by weight of other stereoisomers.

In the section above relating to the preparation of the quinazoline derivative of Formula I, the expression “inert solvent” refers to a solvent which does not react with the starting materials, reagents, intermediates or products in a manner which adversely affects the yield of the desired product.

Persons skilled in the art will appreciate that, in order to obtain compounds of the invention in an alternative and in some occasions, more convenient manner, the individual process steps mentioned hereinbefore may be performed in different order, and/or the individual reactions may be performed at different stage in the overall route (i.e. chemical transformations may be performed upon different intermediates to those associated hereinbefore with a particular reaction).

Certain novel intermediates utilised in the above processes are provided as a further feature of the present invention together with the process for their preparation. Thus the invention further provides a compound of formula (II), (IV), (V), (VII), (XX) and (XXI) as defined above. In particular in these compounds, the group of sub-formula (i)

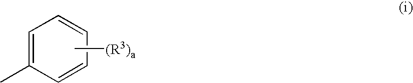

is 3-chloro-2-fluorophenyl or 3-bromo-2-fluorophenyl.

Compounds of formulae (VI), (VIII), (X) and (IX) are either known compounds or they can be prepared from known compounds by conventional methods.

### Biological Assays

The following assays may be used to measure the effects of the compounds of the present invention as inhibitors of the erb-tyrosine kinases, as inhibitors in-vitro of the proliferation of KB cells (human naso-pharangeal carcinoma cells) and as inhibitors in vivo on the growth in nude mice of xenografts of LoVo tumour cells (colorectal adenocarcinoma).

### a) Protein Tyrosine Kinase Phosphorylation Assays

This test measures the ability of a test compound to inhibit the phosphorylation of a tyrosine containing polypeptide substrate by an EGFR, erbB2 or erbB4 tyrosine kinase enzyme.

Recombinant intracellular fragments of EGFR, erbB2 and erbB4 (accession numbers X00588, X03363 and L07868 respectively) were cloned and expressed in the baculovirus/Sf21 system. Lysates were prepared from these cells by treatment with ice-cold lysis buffer (20 mM N-2-hydroxyethylpiperizine-N′-2-ethanesulfonic acid (HEPES) pH7.5, 150 mM NaCl, 10% glycerol, 1% Triton X-100, 1.5 mM MgCl₂, 1 mM ethylene glycol-bis(β-aminoethyl ether) N′,N′,N′,N′-tetraacetic acid (EGTA), plus protease inhibitors and then cleared by centrifugation.

Constitutive kinase activity of the recombinant protein was determined by its ability to phosphorylate a synthetic peptide (made up of a random co-polymer of Glutamic Acid, Alanine and Tyrosine in the ratio of 6:3:1). Specifically, Maxisorb™ 96-well immunoplates were coated with synthetic peptide (0.2 μg of peptide in a 100 μl phosphate buffered saline (PBS) solution and incubated at 4° C. overnight). Plates were washed in PBS-T (phosphate buffered saline with 0.5% Tween 20) then in 50 mM HEPES pH 7.4 at room temperature to remove any excess unbound synthetic peptide. EGFR, ErbB2 or ErbB4 tyrosine kinase activity was assessed by incubation in peptide coated plates for 20 minutes at 22° C. in 100 mM HEPES pH 7.4, adenosine trisphosphate (ATP) at Km concentration for the respective enzyme, 10 mM MnCl₂, 0.1 mM Na₃VO₄, 0.2 mM DL-dithiothreitol (DTI), 0.1% Triton X-100 with test compound in DMSO (final concentration of 2.5%). Reactions were terminated by the removal of the liquid components of the assay followed by washing of the plates with PBS-T.

The immobilised phospho-peptide product of the reaction was detected by immunological methods. Firstly, plates were incubated for 90 minutes at room temperature with anti-phosphotyrosine primary antibodies that were raised in the mouse (4G10 from Upstate Biotechnology). Following extensive washing, plates were treated with Horseradish Peroxidase (HRP) conjugated sheep anti-mouse secondary antibody (NXA931 from Amersham) for 60 minutes at room temperature. After further washing, HRP activity in each well of the plate was measured calorimetrically using 22′-Azino-di-[3-ethylbenzthiazoline sulfonate (6)] diammonium salt crystals (ABTS™ from Roche) as a substrate.

Quantification of colour development and thus enzyme activity was achieved by the measurement of absorbance at 405 nm on a Molecular Devices ThermoMax microplate reader. Kinase inhibition for a given compound was expressed as an IC₅₀ value. This was determined by calculation of the concentration of compound that was required to give 50% inhibition of phosphorylation in this assay. The range of phosphorylation was calculated from the positive (vehicle plus ATP) and negative (vehicle minus ATP) control values.

### b) EGFR Driven KB Cell Proliferation Assay

This assay measures the ability of a test compound to inhibit the proliferation of KB cells (human naso-pharangeal carcinoma obtained from the American Type Culture Collection (ATCC).

KB cells (human naso-pharangeal carcinoma obtained from the ATCC were cultured in Durbecco's modified Eagle's medium (DMEM) containing 10% foetal calf serum, 2 mM glutamine and non-essential amino acids at 37° C. in a 7.5% CO₂ air incubator. Cells were harvested from the stock flasks using Trypsin/ethylaminediaminetetraacetic acid (EDTA). Cell density was measured using a haemocytometer and viability was calculated using trypan blue solution before being seeded at a density of 1.25×10³ cells per well of a 96 well plate in DMEM containing 2.5% charcoal stripped serum, 1 mM glutamine and non-essential amino acids at 37° C. in 7.5% CO₂ and allowed to settle for 4 hours.

Following adhesion to the plate, the cells are treated with or without EGF (final concentration of 1 ng/ml) and with or without compound at a range of concentrations in dimethylsulfoxide (DMSO) (0.1% final) before incubation for 4 days. Following the incubation period, cell numbers were determined by addition of 50 μl of 3-(4,5-Dimethylthiazol-2-yl)-2,5-diphenyltetrazolium bromide (MIN) (stock 5 mg/ml) for 2 hours. MTT solution was then tipped off, the plate gently tapped dry and the cells dissolved upon the addition of 100 μl of DMSO.

Absorbance of the solubilised cells was read at 540 nm using a Molecular Devices ThermoMax microplate reader. Inhibition of proliferation was expressed as an IC₅₀ value. This was determined by calculation of the concentration of compound that was required to give 50% inhibition of proliferation. The range of proliferation was calculated from the positive (vehicle plus EGF) and negative (vehicle minus EGF) control values.

c) Clone 24 phospho-erbB2 Cell Assay

This immunofluorescence end point assay measures the ability of a test compound to inhibit the phosphorylation of erbB2 in a MCF7 (breast carcinoma) derived cell line which was generated by transfecting MCF7 cells with the full length erbB2 gene using standard methods to give a cell line that overexpresses full length wild type erbB2 protein (hereinafter ‘Clone 24’ cells).

Clone 24 cells were cultured in Growth Medium (phenol red free Dulbecco's modified Eagle's medium (DMEM) containing 10% foetal bovine serum, 2 mM glutamine and 1.2 mg/ml G418) in a 7.5% CO₂ air incubator at 37° C. Cells were harvested from T75 stock flasks by washing once in PBS (phosphate buffered saline, pH7.4, Gibco No. 10010-015) and harvested using 2 mls of Trypsin (1.25 mg/ml)/ethylaminediaminetetraacetic acid (EDTA) (0.8 mg/ml) solution. The cells were resuspended in Growth Medium. Cell density was measured using a haemocytometer and viability was calculated using Trypan Blue solution before being further diluted in Growth Medium and seeded at a density of 1×10⁴ cells per well (in 100 ul) into clear bottomed 96 well plates (Packard, No. 6005182).

3 days later, Growth Medium was removed from the wells and replaced with 100 ul Assay Medium (phenol red free DMEM, 2 mM glutamine, 1.2 mg/ml G418) either with or without erbB inhibitor compound. Plates were returned to the incubator for 4 hrs and then 20 μl of 20% formaldehyde solution in PBS was added to each well and the plate was left at room temperature for 30 minutes. This fixative solution was removed with a multichannel pipette, 100 μl of PBS was added to each well and then removed with a multichannel pipette and then 50 μl PBS was added to each well. Plates were then sealed and stored for up to 2 weeks at 4° C.

Immunostaining was performed at room temperature. Wells were washed once with 200 μl PBS/Tween 20 (made by adding 1 sachet of PBS/Tween dry powder (Sigma, No. P3563) to 1 L of double distilled H₂O) using a plate washer then 200 μl Blocking Solution (5% Marvel dried skimmed milk (Nestle) in PBS/Tween 20) was added and incubated for 10 minutes. Blocking Solution was removed using a plate washer and 200 μl of 0.5% Triton X-100/PBS was added to permeabalise the cells. After 10 minutes, the plate was washed with 200 μl PBS/Tween 20 and then 200 μl Blocking Solution was added once again and incubated for 15 minutes. Following removal of the Blocking Solution with a plate washer, 30 μl of rabbit polyclonal anti-phospho ErbB2 IgG antibody (epitope phospho-Tyr 1248, SantaCruz, No. SC-12352-R), diluted 1:250 in Blocking Solution, was added to each well and incubated for 2 hours. Then this primary antibody solution was removed from the wells using a plate washer followed by two 200 μl PBS/Tween 20 washes using a plate washer. Then 30 μl of Alexa-Fluor 488 goat anti-rabbit IgG secondary antibody (Molecular Probes, No. A-11008), diluted 1:750 in Blocking Solution, was added to each well. From now onwards, wherever possible, plates were protected from light exposure, at this stage by sealing with black backing tape. The plates were incubated for 45 minutes and then the secondary antibody solution was removed from the wells followed by two 200 μl PBS/Tween 20 washes using a plate washer. Then 100 μl PBS was added to each plate, incubated for 10 minutes and then removed using a plate washer. Then a further 100 μl PBS was added to each plate and then, without prolonged incubation, removed using a plate washer. Then 50 μl of PBS was added to each well and plates were resealed with black backing tape and stored for up to 2 days at 4° C. before analysis.

The Fluorescence signal is each well was measured using an Acumen Explorer Instrument (Acumen Bioscience Ltd.), a plate reader that can be used to rapidly quantitate features of images generated by laser-scanning. The instrument was set to measure the number of fluorescent objects above a pre-set threshold value and this provided a measure of the phosphorylation status of erbB2 protein. Fluorescence dose response data obtained with each compound was exported into a suitable software package (such as Origin) to perform curve fitting analysis. Inhibition of erbB2 phosphorylation was expressed as an IC₅₀ value. This was determined by calculation of the concentration of compound that was required to give 50% inhibition of erbB2 phosphorylation signal.

### d) In Vivo Xenograft Assay

This assay measures the ability of a test compound to inhibit the growth of a LoVo tumour (colorectal adenocarcinoma obtained from the ATCC) in Female Swiss athymic mice (Alderley Park, nu/nu genotype).

Female Swiss athymic (nu/nu genotype) mice were bred and maintained in Alderley Park in negative pressure Isolators (PFI Systems Ltd.). Mice were housed in a barrier facility with 12 hr light/dark cycles and provided with sterilised food and water ad libitum. All procedures were performed on mice of at least 8 weeks of age. LoVo tumour cell (colorectal adenocarcinoma obtained from the ATCC) xenografts were established in the hind flank of donor mice by sub cutaneous injections of 1×10⁷ freshly cultured cells in 100 μl of serum free media per animal. On day 5 post-implant, mice were randomised into groups of 7 prior to the treatment with compound or vehicle control that was administered once daily at 0.1 ml/10 g body weight. Tumour volume was assessed twice weekly by bilateral Vernier calliper measurement, using the formula (length×width)×√(length×width)×(π/6), where length was the longest diameter across the tumour, and width was the corresponding perpendicular. Growth inhibition from start of study was calculated by comparison of the mean changes in tumour volume for the control and treated groups, and statistical significance between the two groups was evaluated using a Students t test.

e) hERG-Encoded Potassium Channel Inhibition Assay

This assay determines the ability of a test compound to inhibit the tail current flowing through the human ether-a-go-go-related-gene (hERG)-encoded potassium channel.

Human embryonic kidney (BEK) cells expressing the hERG-encoded channel were grown in Minimum Essential Medium Eagle (EMEM; Sigma-Aldrich catalogue number M2279), supplemented with 10% Foetal Calf Serum (Labtech International; product number 4-101-500), 10% Ml serum-free supplement (Egg Technologies; product number 70916) and 0.4 mg/ml Geneticin G418 (Sigma-Aldrich; catalogue number G7034). One or two days before each experiment, the cells were detached from the tissue culture flasks with Accutase (TCS Biologicals) using standard tissue culture methods. They were then put onto glass coverslips resting in wells of a 12 well plate and covered with 2 ml of the growing media.

For each cell recorded, a glass coverslip containing the cells was placed at the bottom of a Perspex chamber containing bath solution (see below) at room temperature (˜20° C.). This chamber was fixed to the stage of an inverted, phase-contrast microscope. Immediately after placing the coverslip in the chamber, bath solution was perfused into the chamber from a gravity-fed reservoir for 2 minutes at a rate of ˜2 ml/min. After this time, perfusion was stopped.

A patch pipette made from borosilicate glass tubing (GC120F, Harvard Apparatus) using a P-97 micropipette puller (Sutter Instrument Co.) was filled with pipette solution (see hereinafter). The pipette was connected to the headstage of the patch clamp amplifier (Axopatch 200B, Axon Instruments) via a silver/silver chloride wire. The headstage ground was connected to the earth electrode. This consisted of a silver/silver chloride wire embedded in 3% agar made up with 0.85% sodium chloride.

The cell was recorded in the whole cell configuration of the patch clamp technique. Following “break-in”, which was done at a holding potential of −80 mV (set by the amplifier), and appropriate adjustment of series resistance and capacitance controls, electrophysiology software (Clampex, Axon Instruments) was used to set a holding potential (−80 mV) and to deliver a voltage protocol. This protocol was applied every 15 seconds and consisted of a 1 s step to +40 mV followed by a 1 s step to −50 mV. The current response to each imposed voltage protocol was low pass filtered by the amplifier at 1 kHz. The filtered signal was then acquired, on line, by digitising this analogue signal from the amplifier with an analogue to digital converter. The digitised signal was then captured on a computer running Clampex software (Axon Instruments). During the holding potential and the step to +40 mV the current was sampled at 1 kHz. The sampling rate was then set to 5 kHz for the remainder of the voltage protocol.

The compositions, pH and osmolarity of the bath and pipette solution are tabulated below.

| Salt | Pipette (mM) | Bath (mM) |
|---|---|---|
| NaCl | — | 137 |
| KCl | 130 | 4 |
| MgCl₂ | 1 | 1 |
| CaCl₂ | — | 1.8 |
| HEPES | 10 | 10 |
| glucose | — | 10 |
| Na₂ATP | 5 | — |
| EGTA | 5 | — |
| Parameter | Pipette | Bath |
| pH | 7.18-7.22 | 7.40 |
| pH adjustment with | 1M KOH | 1M NaOH |
| Osmolarity (mOsm) | 275-285 | 285-295 |

The amplitude of the hERG-encoded potassium channel tail current following the step from +40 mV to −50 mV was recorded on-line by Clampex software (Axon Instruments). Following stabilisation of the tail current amplitude, bath solution containing the vehicle for the test substance was applied to the cell. Providing the vehicle application had no significant effect on tail current amplitude, a cumulative concentration effect curve to the compound was then constructed.

The effect of each concentration of test compound was quantified by expressing the tail current amplitude in the presence of a given concentration of test compound as a percentage of that in the presence of vehicle.

Test compound potency (IC₅₀) was determined by fitting the percentage inhibition values making up the concentration-effect to a four parameter Hill equation using a standard data-fitting package. If the level of inhibition seen at the highest test concentration did not exceed 50%, no potency value was produced and a percentage inhibition value at that concentration was quoted.

Although the pharmacological properties of the compounds of the Formula I vary with structural change as expected, in general activity possessed by compounds of the Formula I, may be demonstrated at the following concentrations or doses in one or more of the above tests (a), (b) and (c):—

Test (a):—IC₅₀ (for EGFR) in the range, for example, 0.001-10 μM;

Test (b):—IC₅₀ in the range, for example, 0.001-10 μM;

Test (c):—IC₅₀ in the range, for example, 0.01-10 μM;

Test (d):—activity in the range, for example, 1-200 mg/kg/day.

By way of example, using Test (a) (for the inhibition of EGFR tyrosine kinase protein phosphorylation) and Test (b), the KB cell assay described above, representative compounds described in the Examples herein gave the IC₅₀ results shown below in Table A:

TABLE A

| | IC₅₀ (nM) Test (a) | |
|---|---|---|
| | (Inhibition of EGFR | IC₅₀ (nM) Test (b) |
| | tyrosine kinase protein | (EGFR driven KB cell |
| Compound of Example | phosphorylation) | proliferation assay) |
| 20 | 64 | 143 |
| 24 | 20 | 50 |
| 37 | 67 | 160 |
| 41 | 52 | 290 |

According to a further aspect of the invention there is provided a pharmaceutical composition which comprises a quinazoline derivative of the Formula I, or a pharmaceutically-acceptable thereof, as defined hereinbefore in association with a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the Formula I will naturally vary according to the nature and severity of the conditions, the age and sex of the animal or patient and the route of administration, according to well known principles of medicine.

In using a compound of the Formula I for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration is however preferred, particularly in tablet form. Typically, unit dosage forms will contain about 0.5 mg to 0.5 g of a compound of this invention.

We have found that the compounds of the present invention possess anti-proliferative properties such as anti-cancer properties that are believed to arise from their erbB family receptor tyrosine kinase inhibitory activity, particularly inhibition of the EGF receptor (erbB1) tyrosine kinase. Furthermore, certain of the compounds according to the present invention possess substantially better potency against the EGF receptor tyrosine kinase, than against other tyrosine kinase enzymes, for example erbB2, VEGF or KDR receptor tyrosine kinases. Such compounds possess sufficient potency against the EGF receptor tyrosine kinase that they may be used in an amount sufficient to inhibit EGF receptor tyrosine kinase whilst demonstrating little, or significantly lower, activity against other tyrosine kinase enzymes such as erbB2. Such compounds are likely to be useful for the selective inhibition of EGF receptor tyrosine kinase and are likely to be useful for the effective treatment of, for example EGF driven tumours.

Accordingly, the compounds of the present invention are expected to be useful in the treatment of diseases or medical conditions mediated alone or in part by erbB receptor tyrosine kinases (especially EGF receptor tyrosine kinase), i.e. the compounds may be used to produce an erbB receptor tyrosine kinase inhibitory effect in a warm-blooded animal in need of such treatment. Thus the compounds of the present invention provide a method for the treatment of malignant cells characterised by inhibition of one or more of the erbB family of receptor tyrosine kinases. Particularly the compounds of the invention may be used to produce an anti-proliferative and/or pro-apoptotic and/or anti-invasive effect mediated alone or in part by the inhibition of erbB receptor tyrosine kinases. Particularly, the compounds of the present invention are expected to be useful in the prevention or treatment of those tumours that are sensitive to inhibition of one or more of the erbB receptor tyrosine kinases, such as EGF and/or erbB2 and/or erbB4 receptor tyrosine kinases (especially EGF receptor tyrosine kinase) that are involved in the signal transduction steps which drive proliferation and survival of these tumour cells. Accordingly the compounds of the present invention are expected to be useful in the treatment of psoriasis, benign prostatic hyperplasia (BPH), atherosclerosis and restenosis and/or cancer by providing an anti-proliferative effect, particularly in the treatment of erbB receptor tyrosine kinase sensitive cancers. Such benign or malignant tumours may affect any tissue and include non-solid tumours such as leukaemia, multiple myeloma or lymphoma, and also solid tumours, for example bile duct, bone, bladder, brain/CNS, breast, colorectal, endometrial, gastric, head and neck, hepatic, lung, neuronal, oesophageal, ovarian, pancreatic, prostate, renal, skin, testicular, thyroid, uterine and vulval cancers.

According to this aspect of the invention there is provided a quinazoline derivative of the Formula I, or a pharmaceutically acceptable salt thereof, for use as a medicament.

According to a further aspect of the invention there is provided a quinazoline derivative of the Formula I, or a pharmaceutically acceptable salt thereof, for use in the production of an anti-proliferative effect in a warm-blooded animal such as a human.

Thus according to this aspect of the invention there is provided the use of a quinazoline derivative of the Formula I, or a pharmaceutically-acceptable salt thereof, as defined hereinbefore in the manufacture of a medicament for use in the production of an anti-proliferative effect in a warm-blooded animal such as a human.

According to a further feature of this aspect of the invention there is provided a method for producing an anti-proliferative effect in a warm-blooded animal, such as a human, in need of such treatment which comprises administering to said animal an effective amount of a quinazoline derivative of the Formula I, or a pharmaceutically acceptable salt thereof, as hereinbefore defined.

According to a further aspect of the invention there is provided the use of a quinazoline derivative of the Formula I, or a pharmaceutically-acceptable salt thereof, as defined hereinbefore in the manufacture of a medicament for use in the prevention or treatment of those tumours which are sensitive to inhibition of erbB receptor tyrosine kinases, such as EGFR and/or erbB2 and/or erbB4 (especially EGFR) tyrosine kinases, that are involved in the signal transduction steps which lead to the proliferation of tumour cells.

According to a further feature of this aspect of the invention there is provided a method for the prevention or treatment of those tumours in a warm-blooded animal such as a human which are sensitive to inhibition of one or more of the erbB family of receptor tyrosine kinases, such as EGFR and/or erbB2 and/or erbB4 (especially EGFR) tyrosine kinases, that are involved in the signal transduction steps which lead to the proliferation and/or survival of tumour cells which comprises administering to said animal an effective amount of a quinazoline derivative of the Formula I, or a pharmaceutically-acceptable salt thereof, as defined hereinbefore.

According to a further feature of this aspect of the invention there is provided a compound of the Formula I, or a pharmaceutically acceptable salt thereof, for use in the prevention or treatment of those tumours in a warm-blooded animal such as a human which are sensitive to inhibition of erbB receptor tyrosine kinases, such as EGFR and/or erbB2 and/or erbB4 (especially EGFR) tyrosine kinases, that are involved in the signal transduction steps which lead to the proliferation of tumour cells.

According to a further aspect of the invention there is provided the use of a quinazoline derivative of the Formula I, or a pharmaceutically-acceptable salt thereof, as defined hereinbefore in the manufacture of a medicament for use in providing a EGFR and/or erbB2 and/or erbB4 (especially a EGFR) tyrosine kinase inhibitory effect in a warm-blooded animal such as a human.

According to a further feature of this aspect of the invention there is provided a method for providing a EGFR and/or an erbB2 and or an erbB4 (especially a EGFR) tyrosine kinase inhibitory effect in a warm-blooded animal such as a human which comprises administering to said animal an effective amount of a quinazoline derivative of the Formula I, or a pharmaceutically-acceptable salt thereof, as defined hereinbefore.

According to a further feature of this aspect of the invention there is provided a compound of the Formula I, or a pharmaceutically acceptable salt thereof, for use in providing a EGFR and/or erbB2 and/or erbB4 (especially a EGFR) tyrosine kinase inhibitory effect in a warm-blooded animal such as a human.

According to a further feature of the present invention there is provided the use of a quinazoline derivative of the Formula I, or a pharmaceutically-acceptable salt thereof, as defined hereinbefore in the manufacture of a medicament for use in providing a selective EGFR tyrosine kinase inhibitory effect in a warm-blooded animal such as a human.

According to a further feature of this aspect of the invention there is provided a method for providing a selective EGFR tyrosine kinase inhibitory effect in a warm-blooded animal such as a human which comprises administering to said animal an effective amount of a quinazoline derivative of the Formula I, or a pharmaceutically-acceptable salt thereof, as defined hereinbefore.

According to a further feature of this aspect of the invention there is provided a compound of the Formula I, or a pharmaceutically acceptable salt thereof, for use in providing a selective EGFR tyrosine kinase inhibitory effect in a warm-blooded animal such as a human.

By “a selective EGFR kinase inhibitory effect” is meant that the quinazoline derivative of Formula I is more potent against EGF receptor tyrosine kinase than it is against other kinases. In particular some of the compounds according to the invention are more potent against EGF receptor kinase than against other tyrosine kinases such as other erbB receptor tyrosine kinases such erbB2. For example a selective EGFR kinase inhibitor according to the invention is at least 5 times, preferably at least 10 times more potent against EGF receptor tyrosine kinase than it is against erbB2 tyrosine kinase, as determined from the relative IC₅₀ values in suitable assays. For example, by comparing the IC₅₀ value from the KB cell assay (a measure of the EGFR tyrosine kinase inhibitory activity) with the IC₅₀ value from the Clone 24 phospho-erbB2 cell assay (a measure of erb-B2 tyrosine kinase inhibitory activity) for a given test compound as described above.

According to a further aspect of the present invention there is provided the use of a quinazoline derivative of the Formula I, or a pharmaceutically-acceptable salt thereof, as defined hereinbefore in the manufacture of a medicament for use in the treatment of a cancer (for example a cancer selected from leukaemia, multiple myeloma, lymphoma, bile duct, bone, bladder, brain/CNS, breast, colorectal, endometrial, gastric, head and neck, hepatic, lung, neuronal, oesophageal, ovarian, pancreatic, prostate, renal, skin, testicular, thyroid, uterine and vulval cancer) in a warm-blooded animal such as a human.

According to a further feature of this aspect of the invention there is provided a method for treating a cancer (for example a cancer selected from leukaemia, multiple myeloma, lymphoma, bile duct, bone, bladder, brain/CNS, breast, colorectal, endometrial, gastric, head and neck, hepatic, lung, neuronal, oesophageal, ovarian, pancreatic, prostate, renal, skin, testicular, thyroid, uterine and vulval cancer) in a warm-blooded animal, such as a human in need of such treatment, which comprises administering to said animal an effective amount of a quinazoline derivative of the Formula I, or a pharmaceutically-acceptable salt thereof, as defined hereinbefore.

According to a further aspect of the invention there is provided a compound of the Formula I, or a pharmaceutically acceptable salt thereof, for use in the treatment of a cancer (for example selected from leukaemia, multiple myeloma, lymphoma, bile duct, bone, bladder, brain/CNS, breast, colorectal, endometrial, gastric, head and neck, hepatic, lung, neuronal, oesophageal, ovarian, pancreatic, prostate, renal, skin, testicular, thyroid, uterine and vulval cancer) in a warm-blooded animal such as a human.

As mentioned above the size of the dose required for the therapeutic or prophylactic treatment of a particular disease will necessarily be varied depending upon, amongst other things, the host treated, the route of administration and the severity of the illness being treated.

The anti-proliferative treatment/tyrosine kinase inhibitory effect/anti-cancer treatment defined hereinbefore may be applied as a sole therapy or may involve, in addition to the compound of the invention, conventional surgery or radiotherapy or chemotherapy. Such chemotherapy may include one or more of the following categories of anti-tumour agents:—

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyridines like 5-fluorouracil and tegafur, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), UH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, MEK inhibitors, tyrosine kinase inhibitors and serine/threonine kinase inhibitors, for example other inhibitors of the epidermal growth factor family (for example other EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO00/40529, WO 00/41669, WO01/92224, WO02/04434 and WO02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.
(x) Cell cycle inhibitors including for example CDK inhibitiors (eg flavopiridol) and other inhibitors of cell cycle checkpoints (eg checkpoint kinase); inhibitors of aurora kinase and other kinases involved in mitosis and cytokinesis regulation (eg mitotic kinesins); and histone deacetylase inhibitors

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a pharmaceutical product comprising a quinazoline derivative of the Formula I as defined hereinbefore and an additional anti-tumour agent as defined hereinbefore for the conjoint treatment of cancer.

Although the compounds of the Formula I are primarily of value as therapeutic agents for use in warm-blooded animals (including man), they are also useful whenever it is required to inhibit the effects of the erbB receptor tyrosine protein kinases. Thus, they are useful as pharmacological standards for use in the development of new biological tests and in the search for new pharmacological agents.

The invention will now be illustrated by the following non limiting examples in which, unless stated otherwise:

(i) temperatures are given in degrees Celsius (° C.); operations were carried out at room or ambient temperature, that is, at a temperature in the range of 18-25° C.;
(ii) organic solutions were dried over anhydrous magnesium sulfate; evaporation of solvent was carried out using a rotary evaporator under reduced pressure (6004000 Pascals; 4.5-30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC and/or analytical LCMS, and reaction times are given for illustration only;
(v) final products had satisfactory proton nuclear magnetic resonance (NMR) spectra and/or mass spectral data;
(vi) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 300 MHz or 400 MHz using perdeuterio dimethyl sulfoxide (DMSO-d₆) as solvent unless otherwise indicated; the following abbreviations have been used: s, singlet; d, doublet; t, triplet; q, quartet; m, multiplet; b, broad;
(viii) chemical symbols have their usual meanings; SI units and symbols are used;
(ix) solvent ratios are given in volume:volume (v/v) terms; and
(x) mass spectra (MS) were run using a Waters or Micromass electrospray LC-MS in positive or negative ion mode; values for m/z are given; generally, only ions which indicate the parent mass are reported; and unless otherwise stated, the mass ion quoted is (MH)⁺;
(xi) unless stated otherwise compounds containing an asymmetrically substituted carbon and/or sulfur atom have not been resolved;
(xii) where a synthesis is described as being analogous to that described in a previous example the amounts used are the millimolar ratio equivalents to those used in the previous example;
(xiii) where compounds were purified using Mass-Triggered Preparative LCMS the following conditions were used:

Column: ThermoHypersil Keystone B-Basic 5λ 21 mm×100 mm

Eluant: 7.5 minutes Gradient from 20% to 95% of acetonitrile in water (buffer

2 g/l of (NH₄)₂CO₃, pH 8.9).

Flow rate: 25 ml/min;

(xiv) the following abbreviations have been used:
DMSO dimethylsulfoxide;
DMF N,N-dimethylformamide;
DMA N,N-dimethylacetamide;
;
HATU O-(7-azabenzotriazol-1-yl)-N,N,N′,N′-tetramethyluronium hexafluorophosphate; and
THF Tetrahydrofuran
   xv) where a synthesis is described as leading to an acid addition salt (e.g. HCl salt), the specific stoichiometry of the salt was not confirmed.

### EXAMPLE 1

### 1-({4-[(3-Chloro-2-fluorophenyl)amino]-7-methoxyquinazolin-6-yl}methyl)-L-proilnamide (Compound No 2 in Table 1)

### (Process (a))

Sodium triacetoxy borohydride (624 mg) was added to a stirred suspension of 4-(3-chloro-2-fluoroanilino)-6-carbaldehyde-7-methoxy quinazoline (650 mg) and L-prolinamide (246 mg) in THF (5 mL) at ambient temperature under a nitrogen atmosphere. After 18 hours the reaction mixture was filtered and evaporated under reduced pressure. The residues were partitioned between saturated sodium bicarbonate solution and methylene chloride. The organics were dried (MgSO₄), filtered and evaporated. The crudes were purified by column chromatography eluting with increasingly polar mixtures of methylene chloride/methanol (100/0 to 90/10). Fractions containing the desired product were combined and evaporated under reduced pressure to give a white foam which was triturated with diethyl ether to a white solid. This was collected by filtration and dried to give the title product (224.6 mg, 27%); ¹H NMR Spectrum: (DMSO d₆) 1.60-1.85 (m, 3H), 2.0-2.25 (m, 1H), 2.25-2.45 (m, 1H), 2.90-3.00 (m, 1H), 3.00-3.15 (m, 1H), 3.60 (d, 1H), 3.85-4.05 (m, 4H), 7.17 (bs, 1H), 7.20 (s, 1H), 7.27 (m, 1H), 7.39 (bs, 1H), 7.42-7.61 (m, 2H), 8.37 (s, 1H), 8.42 (s, 1H), 9.78 (s, 1H); Mass Spectrum: (M+H)⁺ 430.08.

The 4-(3-chloro-2-fluoroanilino)-6-carbaldehyde-7-methoxyquinazoline used a starting was prepared as follows:

A suspension of 4-(3-chloro-2-fluoroanilino)-6-hydroxy-7-methoxyquinazoline (800 mg) in methylene chloride (150 ml) was cooled to 0° C. and to it added pyridine (1.5 ml). Triflic anhydride (507 μl) was then added dropwise and the resulting solution left to stir to ambient temperature. After 18 hours the reaction mixture was washed with water and brine, dried over MgSO₄, filtered and evaporated under reduced pressure. The residues were then triturated with methylene chloride to give 4-(3-chloro-2-fluoroanilino)-6-trifluoromethanesulfonyloxy-7-methoxyquinazoline as a white solid which was collected by filtration and dried (880 mg, 79%); ¹H NMR Spectrum: (DMSO d₆) 4.13 (s, 3H), 7.37 (m, 1H), 7.56 (m, 1H), 7.64 (m, 1H), 7.66 (s, 1H), 8.86 (s, 1H), 9.06 (s, 1H), 11.7 (bs, 1H). Mass Spectrum: (M+H)⁺ 452.

A mixture of 4-(3-chloro-2-fluoroanilino)-6-trifluoromethanesulfonyloxy-7-methoxyquinazoline (883 mg), palladium acetate (14 mg), 1,3 bis diphenylphosphinopropane (25 mg) and triethylamine (543 μl) in methanol (120 ml) and DMF (6 ml) was heated at 70° C. under CO (10 Bar) for 2 hours. The reaction mixture was evaporated under reduced pressure and the residues were purified by flash chromatography on silica eluting with methylene chloride/methanol/saturated NH₃ (aq) 100/8/1. The desired product fractions were combined and reduced in vacuo to give 4-(3-chloro-2-fluoroanilino)-7-methoxy-6-quinazoline carboxylic acid methyl ester as a white solid (630 mg, 89%); ¹H NMR Spectrum: (DMSO 46) 3.89 (s, 3H), 3.98 (s, 3H), 7.29 (m, 1H), 7.32 (s, 1H) 7.51 (m, 2M), 8.51 (s, 1H), 8.85 (s, 1H), 10.15 (s, 1H); Mass Spectrum: (M+H)⁺ 362.

Red-Al (65% in hexanes) (245 μl) was added dropwise to a stirred, cooled (−70° C.) solution of 4-(3-chloro-2-fluoroanilino)-7-methoxy-6-quinazoline carboxylic acid methyl ester (200 mg) in THF (5 ml). After 2 hours the mixture was treated with a further (245 μl) Red-Al (65% in hexanes), then allowed to warm to ambient temperature and stir for 18 hours. The reaction mixture was quenched by the dropwise addition of a solution of sodium hydrogen tartarate (1 g) in water (20 ml). The resulting solids were collected by filtration and washed with water and acetone to give 4-(3-chloro-2-fluoroanilino)-7-methoxy-6-quinazoline methanol as a white powder (220 mg); Mass Spectrum: (M+H)⁺ 334.

A mixture of 4-(3-chloro-2-fluoroanilino)-7-methoxy-6-quinazoline methanol (180 mg) and manganese (IV) oxide (405 mg) in methylene chloride (15 ml) was stirred at ambient temperature for 18 hours. The reaction mixture was then applied directly to a silica column and eluted with 5% methanol/methylene chloride. The fractions containing the desired product were combined and reduced in vacuo to give 4-(3-chloro-2-fluoroanilino)-6-carbaldehyde-7-methoxy quinazoline as a white solid (40 mg); ¹H NMR Spectrum: (DMSO d₆) 4.07 (s, 3H), 7.29 (t, 1H), 7.35 (s, 1H), 7.45-7.60 (m, 2H), 8.50 (s, 1H), 8.96 (s, 1H), 10.35 (s, 1H), 10.43 (s, 1H); Mass Spectrum: (M+H)⁺ 332.

The 4-(3-chloro-2-fluoroanilino)-6-hydroxy-7-methoxyquinazoline used as the starting material in the above reaction can be prepared using conventional methods, for example using the an analogous method to that described in WO97/30034 (example 32 therein) for the preparation of 4-(3-chloro-4-fluoroanilino)-6-hydroxy-7-methoxyquinazoline using 3-chloro-2-fluoroaniline in place of 3-chloro-4-fluoroaniline, for example as described below:

6-Acetoxy-4-chloro-7-methoxyquinazoline (prepared as described in Example 25-5 of in WO01/66099, 6.00 g, 23.8 mmol) and 3-chloro-2-fluoroaniline (3.46 g, 23.8 mmol) were suspended in iso-propanol (200 ml). The mixture was heated to 80° C. under reflux for 3 hours. The solvent was evaporated; the residue was crystallised from acetonitrile, giving 6-acetoxy-4-(3-chloro-2-fluoroanilino)-7-methoxyquinazoline hydrochloride as a pale pink crystalline solid (8.16 g, 92%); ¹H NMR: 2.37 (s, 3H), 4.00 (s, 3H), 7.34 (ddd, 1H), 7.48 (s, 1H), 7.52 (ddd, 1H), 7.61 (ddd, 1H), 8.62 (s, 1H), 8.86 (s, 1H); Mass Spectrum: 362.4, 364.4.

6-Acetoxy-4-(3-chloro-2-fluoroanilino)-7-methoxyquinazoline hydrochloride (8.72 g, 21.9 mmol) was dissolved in methanol (200 ml). Concentrated aqueous ammonia (15 ml) was added, and the solution heated to 50° C. with stirring for 2 hours, causing precipitation of a cream coloured solid. The solid was collected by filtration, washed with diethyl ether (3×200 ml), and dried in vacuo at 60° C. over diphosphorous pentoxide, giving 4-(3-chloro-2-fluoroanilino)-6-hydroxy-7-methoxyquinazoline as an off white solid (5.40 g, 77%); ¹H NMR: 3.95 (s, 3H), 7.19 (s, 1H), 7.23 (dd, 1H), 7.42 (dd, 1H), 7.50 (dd, 1H), 7.64 (s, 1H), 8.32 (s, 1H), 9.43 (s, 1H), 9.67 (br.s, 1H); Mass Spectrum: 320.4, 322.4.

### EXAMPLE 2

### 1-({4-[(3-Chloro-2-fluorophenyl)amino]-7-methoxyquinazolin-6-yl}methyl)-D-prolinamide (Compound No 1 in Table 1)

### (Process (a))

Sodium triacetoxy borohydride (480 mg) was added to a stirred suspension of 4-(3-chloro-2-fluoroanilino)-6-carbaldehyde-7-methoxyquinazoline (500 mg) and D-prolinamide (190 mg) in THF (50 ml) at ambient temperature under a nitrogen atmosphere. After 18 hours the reaction mixture was filtered and evaporated under reduced pressure. The residues were partitioned between saturated sodium bicarbonate solution and methylene chloride. The organics were dried (MgSO₄), filtered and evaporated. The crudes were purified by column chromatography eluting with increasingly polar mixtures of methylene chloride/methanol (100/0 to 90/10). Fractions containing the desired product were combined and evaporated under reduced pressure to give a gum. This was then re-purified by column chromatography eluting with increasingly polar mixtures of ethyl acetate/methanol (100/0 to 90/10). Fractions containing the desired product were combined and evaporated under reduced pressure to give a white foam which was triturated with diethyl ether to give the title compound as a white solid. This was collected by filtration and dried to give the title product (214.3 mg, 33%); ¹H NMR Spectrum: (DMSO d₆) 1.60-1.85 (m, 3H), 2.0-2.20 (m, 1H), 2.20-2.41 (m, 1H), 2.90-3.01 (m, 1H), 3.01-3.10 (m, 1H), 3.60 (d, 1H), 3.85-4.05 (m, 4H), 7.15 (m, 1H), 7.21 (s, 1H), 7.29 (m, 1H), 7.39 (m, 1H), 7.42-7.60 (m, 2H), 8.38 (s, 1H), 8.42 (s, 1H), 9.78 (s, 1H); Mass Spectrum: (M+H)⁺ 429.96.

### EXAMPLE 3

### (4R)-1-({4-[(3-Chloro-2-fluorophenyl)amino]-7-methoxyquinazolin-6-yl}methyl)-4-hydroxy-L-prolinamide (Compound No 4 in Table 1)

### Process (a)

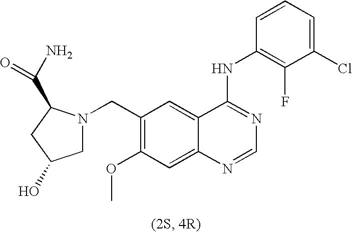

4-[(3-Chloro-2-fluorophenyl)amino]-7-methoxyquinazoline-6-carbaldehyde (50 mg, 0.75 mmol) and (4R)-4-hydroxyl-prolinamide (147 mg, 1.13 mmol) were stirred in 5% acetic acid in dichloromethane (15 ml) and sodium triacetoxyborohydride (240 mg, 1.13 mmol) added portionwise over 0.5 hours. After the final addition the reaction mixture was stirred for 1 hour and then washed with 2N sodium hydroxide. The aqueous layer was adjusted to pH 7-8 and extracted with ethyl acetate and the combined organic layers dried (MgSO₄) and concentrated under reduced pressure. Column chromatography of the residue (5% 7N ammonia in methanol/dichloromethane) gave the title product as a white powder (216 mg, 64%); ¹H NMR Spectrum: (DMSO d₆) 1.88 (m, 1H), 2.01 (m, 1H), 2.33 (dd, 1H), 3.18 (dd, 1H), 3.32 (t, 1H), 3.69 (d, 1H), 3.96 (m, 4H), 4.19 (m, 1H), 4.86 (d, 1H), 7.16 (d, 1H), 7.21 (s, 1H), 7.29 (dt, 1H), 7.36 (d, 1H), 7.50 (dt, 1H), 7.56 (dt, 1H), 8.39 (s, 1H), 8.44 (s, 1H), 9.79 (s, 1H); Mass Spectrum: (M+H)⁺ 446.

The 4-[(3-chloro-2-fluorophenyl)amino]-7-methoxyquinazoline-6-carbaldehyde used as starting material was prepared as follows:

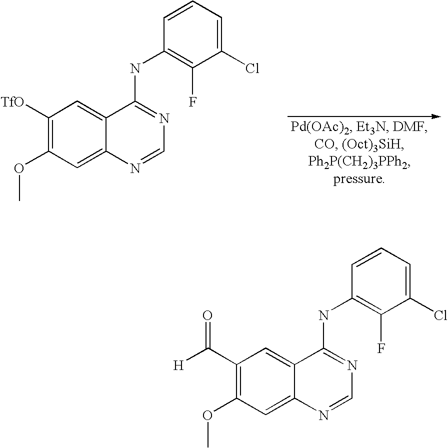

A high pressure vessel was charged with 4-[(3-chloro-2-fluorophenyl)amino]-7-methoxyquinazolin-6-yl trifluoromethanesulfonate (described in Example 1) (10 g, 22.1 mmol), palladium(II)acetate (700 mg, 3.12 mmol), triethylamine (7.6 ml, 54.5 mmol), 1,3-bis diphenylphosphinopropane (1.46 g, 3.54 mmol), trioctylsilane (13.2 ml, 29.4 mmol) and N,N-dimethylformamide (110 ml). The reaction mixture was heated at 70° C. under a carbon monoxide atmosphere (13 Bar) for 3 hours. The mixture was cooled and the lower N,N-dimethylformamide layer was separated, filtered and concentrated under reduced pressure. The residue was suspended in methanol, filtered, washed with isohexane and dried on the filter to give 4-[(3-chloro-2-fluorophenyl)amino]-7-methoxyquinazoline-6-carbaldehyde (3.0 g, 41%) as a pale orange solid; ¹H NMR spectrum: (DMSO d₆) 4.07 (s, 3H), 7.29 (t, 1H), 7.36 (s, 1H), 7.51 (t, 2H), 8.52 (s, 1H), 8.95 (s, 1H), 10.36 (s, 1H), 10.45 (s, 1H); Mass Spectrum: (M+H)⁺ 332.

The (4R)-4-hydroxy-L-prolinamide used as a starting material in Example 3 was prepared as follows:

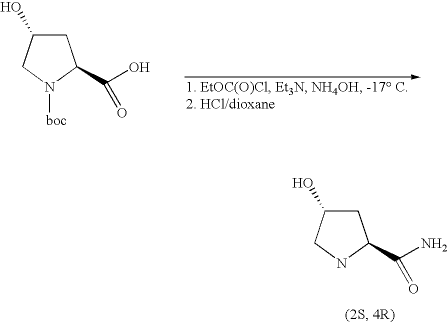

(4R)-1-(tert-Butoxycarbonyl)-4-hydroxy-L-proline (1.0 g, 4.32 mmol) and triethylamine (0.66 ml, 4.76 mmol) in tetrahydrofuran (15 ml) were cooled to −15° C. Ethyl chloroformate (0.45 ml, 4.76 mmol) was added drop wise and then concentrated ammonium hydroxide (1.5 ml). The mixture was stirred at 0 to 5° C. for 2 hours. Saturated ammonium chloride solution was added and the layers separated. The aqueous layer was re-extracted with tetrahydrofuran and the combined organics dried (MgSO₄) and concentrated under reduced pressure. Trituration of the residue with ether gave a white solid (610 mg). The solid was stirred in 4M hydrogen chloride in dioxane (10 ml) for 1 hour and then concentrated under reduced pressure. The residue was dissolved in methanol, absorbed onto an Isolute® SCX column, washed with methanol and eluted with 7N ammonia in methanol to give (4R)-4-hydroxy-L-prolinamide (320 mg, 55%) as a white, crystalline solid; ¹H NMR spectrum: (DMSO d₆) 1.67 (ddd, 1H) 1.90 (qt, 1H), 2.70 (dt, 1H), 2.86 (dd, 1H), 3.63 (t, 1H), 4.16 (m, 1H), 4.63 (brs, 1H), 6.94 (brs, 1H), 7.34 (brs, 1H).

### EXAMPLE 4

### (4S)-1-({4-[(3-Chloro-2-fluorophenyl)amino]-7-methoxyquinazolin-6-yl}methyl)-4-hydroxy-L-prolinamide (Compound 5 in Table 1)

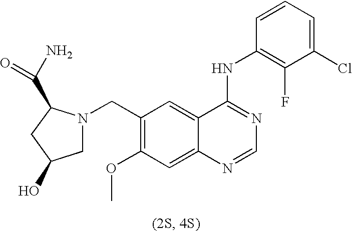

The compound was made by coupling 4-[(3-chloro-2-fluorophenyl)amino]-7-methoxyquinazoline-6-carbaldehyde and (4S)-4-hydroxy-L-prolinamide using an analogous process to that described in for Example 3; ¹H NMR spectrum: (DMSO d₆) 1.69 (ddd, 1H), 2.40 (ddd, 1H), 2.53 (m, 1H), 2.84 (d, 1H), 3.06 (dd, 1H), 3.57 (d, 1H), 3.97 (m, 4H), 4.19 (m, 1H), 4.69 (d, 1H), 7.14 (d, 1H), 7.22 (s, 1H), 7.29 (dt, 1H), 7.44 (d, 1H), 7.50 (dt, 1H), 7.57 (dt, 1H), 8.37 (s, 1H), 8.45 (s, 1H), 9.77 (s, 1H); Mass spectrum: (M+H)⁺ 446.

The (4S)-4-hydroxy-L-prolinamide starting material was prepared as follows:

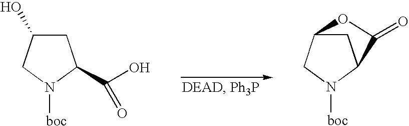

(4R)-1-(tert-Butoxycarbonyl)-4-hydroxy-L-proline (1.00 g, 4.32 mmol) and triphenylphosphine (1.36 g, 5.19 mmol) were stirred in dichloromethane (50 ml) and cooled to 0° C. Diethyl azodicarboxylate (0.8 ml, 5.19 mmol) was slowly added and the mixture allowed to stir at room temperature over night. The mixture was concentrated under reduced pressure. Column chromatography of the residue (20:1 dichloromethane/acetone) gave tert-butyl (1S,4S)-3-oxo-2-oxa-5-azabicyclo[2.2.1]heptane-5-carboxylate (615 mg, 67%) as a white, crystalline solid; ¹H N spectrum: (CDCl₃) 1.48 (s, 9H), 2.01 (d, 1H), 2.20 (m, 1H), 3.46 (d, 1H), 3.53 (dd, 1H), 4.55 (brs, 1H), 5.07 (s, 1H).

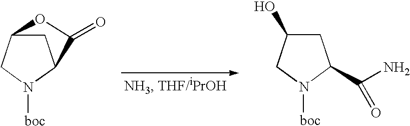

tert-Butyl (1S,4S)-3-oxo-2-oxa-5-azabicyclo[2.2.1]heptane-5-carboxylate (610 mg, 2.86 mmol) was dissolved in tetrahydrofuran (50 ml) and isopropanol (30 ml) and cooled to 0° C. The solution was saturated with ammonia gas, allowed to warm to room temperature and stirred for 48 hours. The mixture was concentrated under reduced pressure to give an oil. Trituration with ether gave (4S)-1-(tert-butoxycarbonyl)-4-hydroxy-L-prolinamide (495 mg, 75%) as a white, crystalline solid; ¹H NMR spectrum: (DMSO d₆ 100° C.) 1.40 (s, 9H), 1.80 (m, 1H), 2.30 (ddd, 1H), 3.24 (m, 1H), 3.50 (dd, 1H), 4.08 (dd, 1H), 4.18 (m, 1H), 4.90 (d, 1H), 6.85 (brs, 2H).

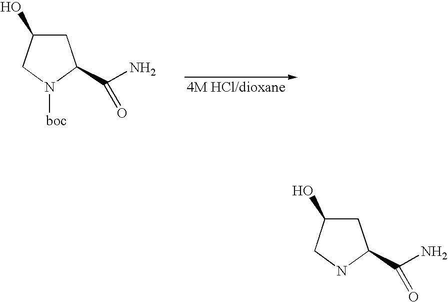

(4S)-1-(tert-Butoxycarbonyl)-4-hydroxy-L-prolinamide (490 mg, 2.13 mmol) was stirred in 4M hydrogen chloride in dioxane (10 ml) for 1 hour and then concentrated under reduced pressure. The residue was dissolved in methanol, absorbed onto an Isolute® SCX column, washed with methanol and eluted with 7N ammonia in methanol to give (4S)-4-hydroxy-L-prolinamide (270 mg, 98%) as a white, crystalline solid; ¹H NMR spectrum: (DMSO d₆) 1.57 (ddd, 1H), 2.12 (ddd, 1H), 2.65 (dd, 1H), 2.83 (dd, 1H), 3.40 (dd, 1H), 4.09 (m, 1H), 4.59 (brs, 1H), 6.92 (brs, 1H), 7.35 (brs, 1H).

### EXAMPLE 5

### (4S)-1-({4-[(3-chloro-2-fluorophenyl)amino]-7-methoxyquinazolin-6-yl}methyl)-4-hydroxy-D-prolinamide (Compound 6 in Table 1)

### (Process (a))

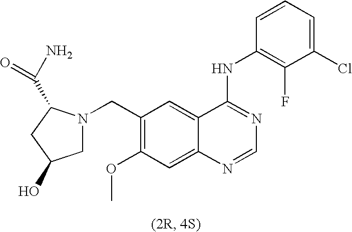

The title compound was made by coupling 4-[(3-chloro-2-fluorophenyl)amino]-7-methoxyquinazoline-6-carbaldehyde and (4S)-4-hydroxy-D-prolinamide using an analogous process to that described in Example 3; ¹H NMR spectrum: (DMSO d₆) 1.88 (m, 1H), 2.01 (m, 1H), 2.33 (dd, 1H), 3.18 (dd, 1H), 3.32 (t, 1H), 3.69 (d, 1H), 3.96 (m, 4H), 4.19 (m, 1H), 4.86 (d, 1H), 7.16 (d, 1H), 7.21 (s, 1H), 7.29 (dt, 1H), 7.36 (d, 1H), 7.50 (dt, 1H), 7.56 (dt, 1H), 8.39 (s, 1H), 8.44 (s, 1H), 9.79 (s, 1H); Mass Spectrum: (M+H)⁺ 446.

The (4S)-4-hydroxy-D-prolinamide used as starting material was prepared using the same methodology as described in the equivalent step in Example 3 using (4S)-1-(tert-butoxycarbonyl)-4-hydroxy-D-proline.

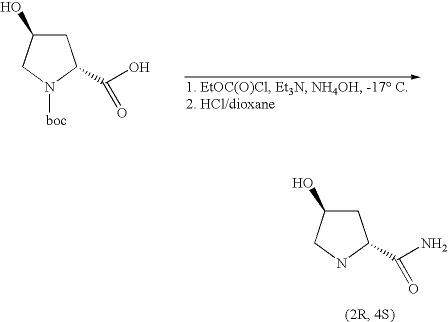

¹H NMR spectrum: (DMSO d₆) 1.67 (ddd, 1H), 1.90 (qt, 1H), 2.70 (dt, 1H), 2.86 (dd, 1H), 3.63 (t, 1H), 4.16 (m, 1H), 4.63 (brs, 1H), 6.94 (brs, 1H), 7.34 (brs, 1H).

### EXAMPLE 6

### (4R)-1-({4-[(3-Chloro-2-fluorophenyl)amino]-7-methoxyquinazolin-6-yl}methyl)-4-hydroxy-D-prolinamide (Compound 7 in Table 1)

### (Process (a))

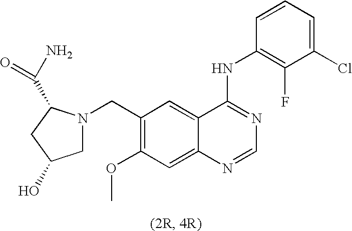

The title compound was made by coupling 4-[(3-chloro-2-fluorophenyl)amino]-7-methoxyquinazoline-6-carbaldehyde and (4R)-4-hydroxy-D-prolinamide using an analogous process to that described in Example 3; ¹H NMR spectrum: (DMSO d₆) 1.69 (ddd, 1H), 2.40 (ddd, 1H), 2.53 (m, 1H), 2.84 (d, 1H), 3.06 (dd, 1H), 3.57 (d, 1H), 3.97 (m, 4H), 4.19 (m, 1H), 4.69 (d, 1H), 7.14 (d, 1H), 7.22 (s, 1H), 7.29 (dt, 1H), 7.44 (d, 1H), 7.50 (dt, 1H), 7.57 (dt, 1H), 8.37 (s, 1H), 8.45 (s, 1H), 9.77 (s, 1H), Mass Spectrum: (M+H)⁺ 446

The (4R)-4-hydroxy-D-prolinamide used as starting material was prepared using the same methodology as described in the equivalent step in Example 5 from (4S)-1-(tert-butoxycarbonyl)-4-hydroxy-D-proline.

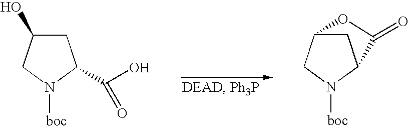

(1R,4R)-3-oxo-2-oxa-5-azabicyclo[2.2.1]heptane-5-carboxylate was prepared using the same methodology as described in the equivalent step in Example 5; ¹H NMR spectrum: (CDCl₃) 1.48 (s, 9H), 2.01 (d, 1H), 2.20 (m, F1H), 3.46 (d, 1H), 3.53 (dd, 1H), 4.55 (brs, 1H), 5.07 (s, 1H).

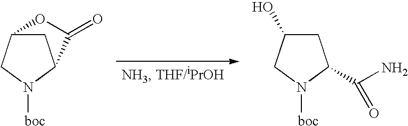

(4R)-1-(tert-butoxycarbonyl)-4-hydroxy-D-prolinamide was prepared using the same methodology as described in the equivalent step in Example 5; ¹H NMR spectrum: (DMSO d₆ 100° C.) 1.40 (s, 9H), 1.80 (m, 1H), 2.30 (ddd, 1H), 3.24 (m, 1H), 3.50 (dd, 1H), 4.08 (dd, 1H), 4.18 (m, 1H), 4.90 (d, 1H), 6.85 (brs, 2H).

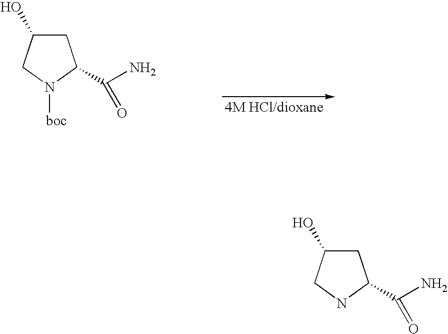

(4R)-4-hydroxy-D-prolinamide was prepared using the same methodology as described in the equivalent step in Example 5; ¹H NMR spectrum: (DMSO d₆) 1.57 (ddd, 1H), 2.12 (ddd, 1H), 2.65 (dd, 1H), 2.83 (dd, 1H), 3.40 (dd, 1H), 4.09 (m, 1H), 4.59 (brs, 1H), 6.92 (brs, 1H), 7.35 (brs, 1H).

### EXAMPLE 7

### 1-({4-[(3-Chloro-4-fluorophenyl)amino]-7-methoxyquinazolin-6-yl}methyl)-L-prolinamide (Compound No 3 in Table 1)

### (Process (a))

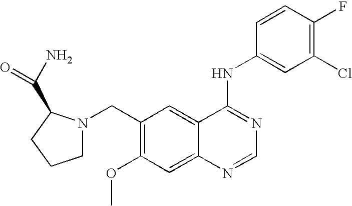

4-[(3-Chloro-4-fluorophenyl)amino]-7-methoxyquinazoline-6-carbaldehyde was coupled with L-prolinamide using an analogous method to that described in Example 1 to give the title product; ¹H NMR Spectrum: (DMSO d₆) 1.55-1.85 (m, 3H), 2.05-2.20 (m, 1H), 2.30-2.50 (m, 1H), 2.85-2.98 (m, 1H), 2.98-3.15 (m, 1H), 3.60 (d, 1H), 3.85-4.10 (m, 4H), 7.14 (bs, 1H), 7.23 (s, 1H), 7.38 (bs, 1H), 7.39 (m, 1H), 7.73-7.88 (m, 1H), 8.05-8.20 (m, 1H), 8.41 (s, 1H), 8.55 (s, 1H), 9.70 (s, 1H); Mass Spectrum: (M+H)⁺ 430.02.

The 4-[(3-chloro-4-fluorophenyl)amino]-7-methoxyquinazoline-6-carbaldehyde used as starting material was prepared as follows:

4-[(3-chloro-4-fluorophenyl)amino]-7-methoxyquinazolin-6-yl trifluoromethanesulfonate was prepared analogously as described in Example 1 (preparation of starting materials) by reacting 4-[(3-chloro-4-fluorophenyl)amino]-7-methoxyquinazolin-6-ol (WO97/30034 Example 32 therein) with triflic anhydride; NMR Spectrum: (DMSO d₆) 4.14 (s, 3H), 7.51 (s, 1H), 7.57 (m, 1H), 7.68 (m, 1H), 8.00 (m, 1H), 8.82 (s, 1H), 8.93 (s, 1H), 11.13 (bs, 1H); Mass Spectrum: (M+H)⁺ 452; (M−H)⁻ 450.

4-[(3-chloro-4-fluorophenyl)amino]-7-methoxyquinazoline-6-carbaldehyde was prepared analogously as described in the equivalent step in Example 3 from 4-[(3-chloro-4-fluorophenyl)amino]-7-methoxyquinazolin-6-yl trifluoromethanesulfonate; NMR Spectrum: (DMSO d₆) 4.03 (s, 3H), 7.30 (s, 1H), 7.43 (m, 1H), 7.73-7.90 (m, 1H), 8.08-8.22 (m, 1H), 8.59 (s, 1H), 8.95 (s, 1H), 10.21 (s, 1H), 10.42 (s, 1H); Mass Spectrum: (M+H)⁺ 332.04; (M−H)⁻ 330.01.

### EXAMPLE 8

### 1-({4-[(3-chloro-4-fluorophenyl)amino]-7-methoxyquinazolin-6-yl}methyl)-D-prolinamide (Compound No 8 in Table 1)

### Process (a)

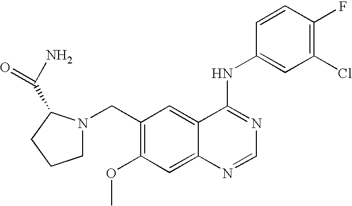

4-[(3-Chloro-4-fluorophenyl)amino]-7-methoxyquinazoline-6-carbaldehyde was coupled with D-prolinamide analogously as for Example 1 to give the title product; ¹H NMR Spectrum: (DMSO 46) 1.55-1.85 (m, 3H), 2.00-2.25 (m, 1H), 2.28-2.50 (m, 1H), 2.82-2.98 (m, 1H), 2.98-3.11 (m, 1H), 3.58 (d, 1H), 3.85-4.10 (m, 4H), 7.15 (bs, 1H), 7.20 (s, 1H), 7.38 (bs, 1H), 7.42 (m, 1H), 7.70-7.90 (m, 1H), 8.05-8.20 (m, 1H), 8.39 (s, 1H), 8.52 (s, 1H), 9.70 (s, 1H); Mass Spectrum: (M+H)⁺ 430.16.

### EXAMPLE 9

### (4R)-1-({4-[(3-chloro-4-fluorophenyl)amino]-7-methoxyquinazolin-6-yl}methyl-4-hydroxy-D-Prolinamide (Compound No 9 in Table 1)

### (Process (a))

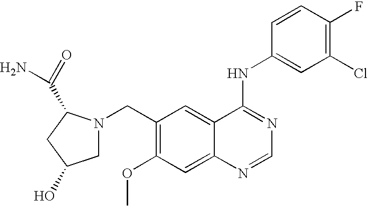

4-[(3-Chloro-4-fluorophenyl)amino]-7-methoxyquinazoline-6-carbaldehyde was coupled with (4R)-4-hydroxy-D-prolinamide (Example 7) analogously as for Example 1 to give the title product; ¹H NMR Spectrum: (DMSO d₆+CD₃COOD) 1.65-1.80 (m, 1H), 2.3-2.55 (m, 1H), 2.60-2.72 (m, 1H), 2.92 (d, 1H), 3.25 (m, 1H), 3.72 (d, 1H), 3.93 (s, 3H), 4.05 (d, 1H), 4.18 (m, 1H), 7.23 (s, 1H), 7.38 (m, 1H), 7.70-7.83 (m, 1H), 8.09 (dd, 1H), 8.39 (s, 1H), 8.55 (s, 1H); Mass Spectrum: (M+H)⁺ 446.02.

### EXAMPLE 10

### (4R)-1-({4-[(3-Chloro-2-fluorophenyl)amino]7-methoxyquinazolin-6-yl}methyl)-4-methoxy-D-prolinamide (Compound 13 in Table 1)

### (Process (a))

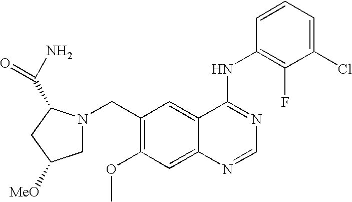

4-[(3-Chloro-2-fluorophenyl)amino]-7-methoxyquinazoline-6-carbaldehyde was coupled with (4R)-4-methoxy-D-prolinamide using an analogous method to that described in Example 3 to give the title product; ¹H NMR spectrum: (DMSO d₆) 1.81 (ddd, 1H), 2.41 (ddd, 1H), 2.47 (m, 1H), 2.98 (d, 1H), 3.09 (m, 4H), 3.58 (d, 1H), 3.88 (m, 1H), 3.97 (m, 4H), 7.14 (d, 1H), 7.23 (s, 1H), 7.30 (t, 1H), 7.37 (d, 1H), 7.50 (t, 1H), 7.57 (t, 1H), 8.38 (s, 1H), 8.45 (s, 1H), 9.81 (s, 1H); Mass Spectrum: (M+H)⁺ 460.0

The (4R)-4-methoxy-D-prolinamide used as the starting material was prepared as follows:

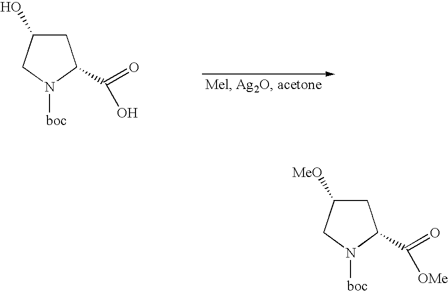

(4R)-1-(tert-Butoxycarbonyl)-4-hydroxy-D-proline (5.0 g, 21.6 mmol) was dissolved in acetone (35 ml) and silver(I) oxide (16.5 g, 71.2 mmol) was added. Further acetone was added (total of 100 ml) in order to allow the solution to stir. Methyl iodide (4.7 ml, 75.7 mmol) was added and the mixture stirred over night. The mixture was filtered and concentrated under reduced pressure. Starting material still remained so the above procedure was repeated. Column chromatography (diethyl ether/acetone, 1:1) gave 1-tert-butyl 2-methyl (2R,4R)-4-methoxypyrrolidine-1,2-dicarboxylate (3.0 g, 54%) as a clear oil; ¹H NMR spectrum: (DMSO d₆ 100° C.) 1.38 (s, 9H), 2.00 (dt, 1H), 2.38 (m, 1H), 3.19 (s, 3H), 3.23 (dd, 1H), 3.57 (dd, 1H), 3.63 (s, 3H), 3.94 (m, 1H), 4.25 (dd, 1H).

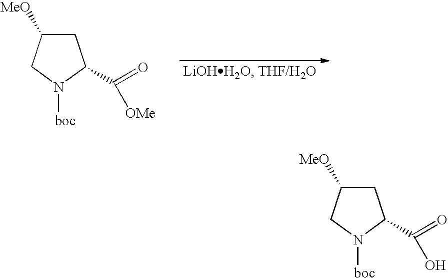

Lithium hydroxide mono-hydrate (1.42 g, 33.7 mmol) was added to 1-tert-butyl 2-methyl (2R,4R)-4-methoxypyrrolidine-1,2-dicarboxylate (1.75 g, 6.75 mmol) in tetrahydrofuran (40 ml) and water (20 ml) and stirred at room temperature for 5 hours. Hydrogen chloride (8.5 ml of a 4M solution in dioxane, 34.0 mmol) was added and the solution concentrated under reduced pressure to remove most of the tetrahydrofuran. The remaining aqueous solution was extracted with dichloromethane which was dried (MgSO₄) and concentrated under reduced pressure to give (4R)-1-(tert-butoxycarbonyl)-4-methoxy-D-proline (1.45 g, 88%) as a white, crystalline solid; ¹H NMR spectrum: (DMSO d₆ 100° C.) 1.39 (s, 9H), 1.99 (dt, 1H), 2.38 (m, 1H), 3.20 (m, 4H), 3.58 (t, 1H), 3.93 (m, 1H), 4.15 (dd, 1H).

(4R)-4-methoxy-D-prolinamide was prepared from (4R)-1-(tert-butoxycarbonyl)-4-methoxy-D-proline by removing the BOC protecting group using an analogous method as for the equivalent step in Example 4; ¹H NMR spectrum: (DMSOd₆) 1.75 (m, 1H), 2.10 (m, 1H), 2.82 (m, 3H), 3.13 (s, 3H), 3.96 (m, 1H), 3.78 (m, 1H), 6.91 (brs, 1H), 7.28 (brs, 1H).

### EXAMPLE 11

### Preparation of (2S)-1-({4-[(3-chloro-2-fluorophenyl)amino]-7-methoxyquinazolin-6-yl}methyl)piperidine-2-carboxamide (Compound No. 15 in Table 2)

### (Process (a))

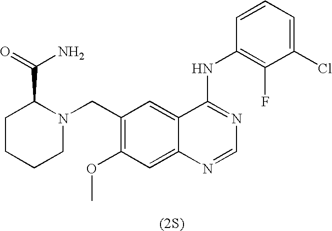

4-[(3-Chloro-2-fluorophenyl)amino]-7-methoxyquinazoline-6-carbaldehyde (250 mg, 0.75 mmol) and (2S)-piperidine-2-carboxamide (145 mg, 1.13 mmol) were stirred in 5% acetic acid in dichloromethane (15 ml) and sodium triacetoxyborohydride (240 mg, 1.13 mmol) added portionwise over 0.5 hours. After the final addition the reaction mixture was stirred for 1 hour and then washed with 2N sodium hydroxide. The aqueous layer was adjusted to pH 7-8 and extracted with ethyl acetate and the combined organic layers dried (MgSO₄) and concentrated under reduced pressure. The residues were purified by flash chromatography eluting with increasingly polar mixtures of methanol/dichloromethane (1-2%) to give the title product as a white powder (106 mg, 32%); ¹H NMR spectrum: (DMSO d₆) 1.31 (m, 1H), 1.47-1.72 (m, 4H), 1.86 (m, 1H), 2.01 (dt, 1H), 2.76 (dd, 1H), 2.88 (m, 1H), 3.42 (d, 1H), 3.77 (d, 1H), 3.95 (s, 3H), 7.12 (s, 1H), 7.20 (m, 2H), 7.30 (dt, 1H), 7.51 (dt, 1H), 7.60 (m, 1H), 8.42 (s, 1H), 8.44 (s, 1H), 9.73 (s, 1H); Mass Spectrum: (M+H)⁺ 444.6.

The (2S)-piperidine-2-carboxamide used as starting material was prepared as follows:

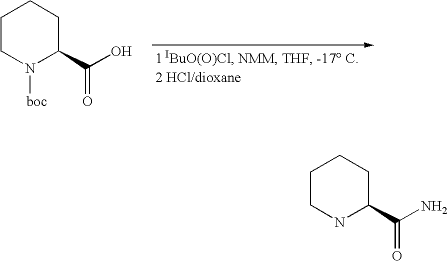

(S)-1-(tert-Butoxycarbonyl)-piperidine-2-carboxylic acid (1.0 g, 4.36 mmol) and N-methylmorpholine (0.53 ml, 4.79 mmol) in tetrahydrofuran (15 ml) were cooled to −15° C. Isobutyl chloroformate (0.44 ml, 4.79 mmol) was added drop wise and then concentrated ammonium hydroxide (1.5 ml). The mixture was stirred at 0 to 5° C. for 2 hours. The mixture was concentrated under reduced pressure and the residue partitioned between ethyl acetate and 10% citric acid. The organic layer was washed with saturated sodium hydrogen carbonate solution, dried (MgSO₄) and concentrated under reduced pressure to give an oil which crystallised on standing (550 mg, 55%). This was used without further purification. The solid was stirred in 4M hydrogen chloride in dioxane (10 ml) for 1 hour and then concentrated under reduced pressure. The residue was dissolved in methanol, absorbed onto an Isolute® SCX column, washed with methanol and eluted with 7N ammonia in methanol to give (2S)-piperidine-2-carboxamide (291 mg, 96%) as a white, crystalline solid; ¹H NMR spectrum: (DMSO d₆) 1.31 (m, 3H), 1.45 (m, 1H), 1.71 (m, 2H), 2.12 (s, 1H), 2.45 (m, 1H), 2.93 (m, 2H), 6.86 (brs, 1H), 7.04 (brs, 1H).

### EXAMPLE 12

### (2R)-1-({4-[(3-Chloro-2-fluorophenyl)amino]-7-methoxyquinazolin-6-yl}methyl)piperidine-2-carboxamide (Compound No 10 in Table II)

### (Process (a))

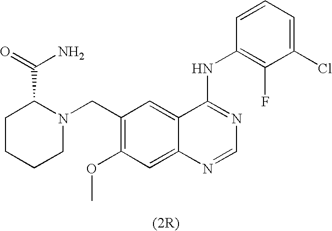

4-[(3-Chloro-2-fluorophenyl)amino]-7-methoxyquinazoline-6-carbaldehyde was coupled with (2R)-piperidine-2-carboxamide analogously as for Example 1 to give the title product; ¹H NMR spectrum: (DMSO d₆) 1.31 (m, 1H), 1.47-1.72 (m, 4H), 1.86 (m, 1H), 2.01 (dt, 1H), 2.76 (dd, 1H), 2.88 (m, 1H), 3.42 (d, 1H), 3.77 (d, 1H), 3.95 (s, 3H), 7.12 (s, 1H), 7.20 (m, 2H), 7.30 (dt, 1H), 7.51 (dt, 1H), 7.60 (m, 1H), 8.42 (s, 1H), 8.44 (s, 1H), 9.73 (s, 1H); Mass Spectrum: (M+H)⁺ 444.6.

The (2R)-piperidine-2-carboxamide used as the starting material was prepared analogously as for the equivalent step in Example 11 starting from tert-butyl (2R)-2-(aminocarbonyl)piperidine-1-carboxylate; ¹H NMR spectrum: (DMSO d₆) 1.31 (m, 3H), 1.45 (m, 1H), 1.71 (m, 2H), 2.12 (s, 1H), 2.45 (m, 1H), 2.93 (m, 2H), 6.86 (brs, 1H), 7.04 (brs, 1H).

### EXAMPLE 13

### 4-({4-[(3-chloro-2-fluorophenyl)amino]-7-methoxyquinazolin-6-yl}methyl)morpholine-3-carboxamide (Compound 11 in Table II)

### (Process (a))

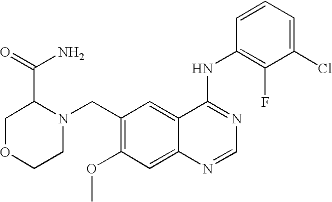

4-[(3-Chloro-2-fluorophenyl)amino]-7-methoxyquinazoline-6-carbaldehyde was coupled with morpholine-3-carboxamide analogously as for Example 1 to give the title product; ¹H NMR spectrum: (DMSO d₆) 2.20 (m, 1H), 2.77 (d, 1H), 2.95 (dd, 1H), 3.48 (m, 3H), 3.72 (d, 1H), 3.86 (m, 2H), 3.95 (s, 3H), 7.21 (s, 1H), 7.31 (m, 3H), 7.52 (m, 2H), 8.39 (s, 1H), 8.43 (s, 1H), 9.75 (s, 1H); Mass Spectrum: (M+H)⁺ 446.5.

The morpholine-3-carboxamide starting material was prepared analogously as for the equivalent step in Example 3 (preparation of starting materials) using from 4-(tert-butoxycarbonyl)morpholine-3-carboxylic acid; ¹H NMR (spectrum): (DMSO d₆) 2.71 (m, 2H), 2.89 (brs, 1H), 3.21 (dd, 1H), 3.35 (m, 2H), 3.58 (dt, 1H), 3.73 (dd, 1H), 7.07 (brs, 1H), 7.21 (brs, 1H).

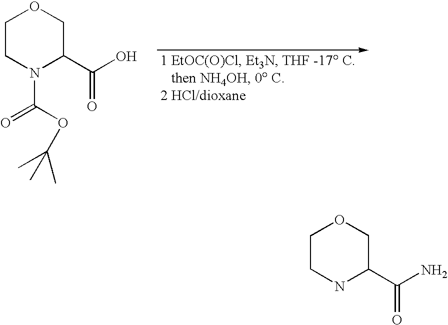

### EXAMPLE 14

### (2R)-1-({4-[(3-Chloro-4-fluorophenyl)amino]-7-methoxyquinazolin-6-yl}methyl)piperidine-2-carboxamide (Compound 12 in Table II)

### (Process (a))

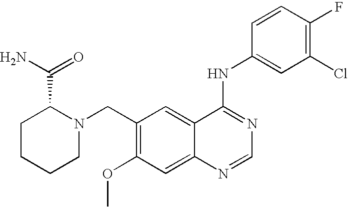

4-[(3-Chloro-4-fluorophenyl)amino]-7-methoxyquinazoline-6-carbaldehyde was coupled with (2R)-piperidine-2-carboxamide (Prepared as described in Example 12) using an analogous process to that described in Example 1 to give the title product; ¹H NMR Spectrum: (DMSO d₆) 1.17-1.75 (m, 4H), 1.75-1.90 (m, 1H), 1.90-2.10 (m, 1H), 2.65-2.80 (m, 1H), 2.80-2.90 (m, 1H), 3.25-3.33 (m, 1H), 3.33-3.42 (m, 1H), 3.85 (d, 1H), 3.95 (s, 3H), 7.12 (s, 1H), 7.20 (s, 2H), 7.45 (m, 1H), 7.70-7.82 (m, 1H), 8.05-8.15 (m, 1H), 8.37 (s, 1H), 8.55 (s, 1H), 9.70 (s, 1H); Mass Spectrum: (M+H)⁺ 444.16, (M−H)⁻ 446.20.

### EXAMPLE 15

### 1-({4-[(3-Chloro-2-fluorophenyl)amino]-7-methoxyquinazolin-6-yl}methyl)-N,N-dimethyl-L-prolinamide (Compound 14 in Table I)

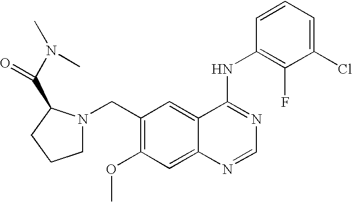

4-[(3-chloro-2-fluorophenyl)amino]-7-methoxyquinazoline-6-carbaldehyde was coupled with N,N-dimethyl-L-prolinamide using an analogous process to that described in Example 1 to give the title product; ¹H NMR Spectrum: (DMSO d₆) 1.60-1.90 (m, 3H), 1.93-2.12 (m, 1H), 2.35-2.60 (m, 1H+DMSO), 2.75 (s, 3H), 2.95 (s, 3H), 3.00-3.15 (m, 1H), 3.53 (dd, 1H), 3.80 (ABq, 2H), 3.92 (s, 3H), 7.15 (s, 1H), 7.25 (m, 1H), 7.40-7.60 (m, 2H), 8.25 (s, 1H), 8.40 (s, 1H), 9.80 (bs, 1H); Mass Spectrum: (M+H)⁺ 458.0, (M−H)⁻ 455.97.

### EXAMPLE 16

### 1-({4-[(3-Chloro-4-fluorophenyl)amino]-7-methoxyquinazolin-6-yl}methyl)-N-ethyl-D-prolinamide

### (Process (c))

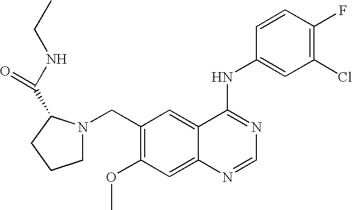

1-Ethyl-3-(3-dimethylaminopropyl)carbodiimide hydrochloride (67 mg) was added to a stirred solution of 1-({-[(3-chloro-4-fluorophenyl)amino]-7-methoxyquinazolin-6-yl}methyl)-D-proline (100 mg), 1-hydroxy benzotriazole, Ethylamine hydrochloride (21.8 mg) and N-methylmorpholine (127 μl) in DMF (5 ml). The reaction mixture was left to stir for 18 hours and evaporated to dryness. The residues were partitioned between saturated aqueous sodium bicarbonate solution (25 ml) and ethyl acetate (2×10 ml). The combined organics were washed with water (10 ml) and brine (10 ml), dried over magnesium sulfate, filtered and evaporated. The crudes were then purified by flash chromatography on silica, eluting with increasingly polar mixtures of methylene chloride/methanol (100/0-90/10). Fractions containing the required product were combined and evaporated to dryness. The resulting foam was triturated with diethyl ether/i-hexane (1/1) to give a white solid which was collected by filtration and dried under vacuum to give the title product (54.2 mg); ¹H NMR Spectrum: (DMSO d₆) 0.93 (t, 3H), 1.50-1.83 (m, 3H), 2.00-2.20 (m, 1H), 2.35-2.60 (m, 1H+DMSO), 2.85-3.00 (m, 1H), 3.00-3.17 (m, 3H), 3.63 (d, 1H), 3.93 (d, 1H), 3.98 (s, 3H), 7.22 (s, 1H), 7.43 (dd, 1H), 7.65-7.75 (m, 1H), 7.75-7.89 (m, 1H), 8.15 (m, 1H), 8.39 (s, 1H), 8.55 (s, 1H), 9.70 (s, 1H); Mass Spectrum: (M+H)⁺ 458.

The 1-({4-[(3-chloro-4-fluorophenyl)amino]-7-methoxyquinazolin-6-yl}methyl)-D-proline starting material was made as follows:

4-[(3-chloro-4-fluorophenyl)amino]-7-methoxyquinazoline-6-carbaldehyde (prepared as described in Example 7-preparation of starting material) was coupled with D-proline using an analogous process to that described in Example 1 to give 1-({4-[(3-chloro-4-fluorophenyl)amino]-7-methoxyquinazolin-6-yl}methyl)-D-proline; ¹H NMR Spectrum: (DMSO d₆) 1.60-2.00 (m, 3H), 2.00-2.25 (m, 1H), 2.60-2.80 (m, 1H), 3.10-3.30 (m, 1H), 3.50 (q, 1H), 3.95 (s, 3H), 4.10 (ABq, 2H), 7.20 (s, 1H), 7.40 (dd, 1H), 7.70-7.90 (m, 1H), 8.15 (dd, 1H), 8.50 (s, 1H), 8.55 (s, 1H), 9.90 (s, 1H); Mass Spectrum: (M+H)⁺ 431.

### EXAMPLE 17

### 1-({4-[(3-Chloro-4-fluorophenyl)amino]-7-methoxyquinazolin-6-yl}methyl)-N-methyl-D-prolinamide

### (Process (c))

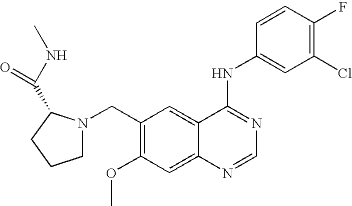

1-({4-[(3-chloro-4-fluorophenyl)amino]-7-methoxyquinazolin-6-yl}methyl)-D-proline was coupled with methylamine hydrochloride using an analogous process to that described in Example 16 to give the title product; ¹H NMR Spectrum: (DMSO d₆) 1.50-1.85 (m, 3H), 2.00-2.20 (m, 1H), 2.35-2.55 (m, 1H+DMSO), 2.63 (d, 3H), 2.82-3.00 (m, 1H), 3.00-3.20 (m, 1H), 3.62 (d, 1H) 3.95 (d, 1H), 3.97 (s, 3H), 7.22 (s, 1H), 7.44 (dd, 1H), 7.65-7.76 (m, 1H), 7.76-7.85 (m, 1H), 8.15 (dd, 1H), 8.39 (s, 1H), 8.57 (s, 1H), 9.68 (s, 1H); Mass Spectrum: (M+H)⁺ 444.

### EXAMPLE 18

### 1-({4-[(3-Chloro-4-fluorophenyl)amino]-7-methoxyquinazolin-6-yl}methyl)-N-cyclopentyl-D-prolinamide

### (Process (c))

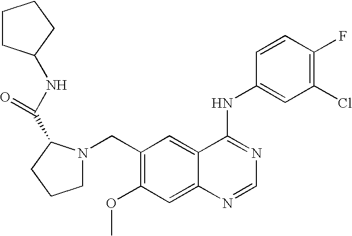

1-({4-[(3-chloro-4-fluorophenyl)amino]-7-methoxyquinazolin-6-yl}methyl)-D-proline was coupled with cyclopentylamine using an analogous process to that described in Example 16 to give the title product; ¹H NMR Spectrum: (DMSO d₆) 1.00-1.90 (m, 7H), 2.00-2.20 (m, 1H), 2.40-2.70 (m, 1H+DMSO), 2.95-3.08 (m, 1H), 3.08-3.20 (m, 1H), 3.65-4.10 (m, 7H), 3.97 (s, 3H), 7.20 (s, 1H), 7.32-7.55 (m, 2H), 7.70-7.90 (m, 1H), 8.05-8.22 (m, 1H), 8.40 (s, 1H), 8.55 (s, 1H), 9.70 (s, 1H); Mass Spectrum: (M+H)⁺ 498.

### EXAMPLE 19

### 1 ({4-[(3-Chloro-4-fluorophenyl)amino]-7-methoxyquinazolin-6-yl}methyl)-N-prop-2-yn-1-yl-D-prolinamide

### (Process (c))

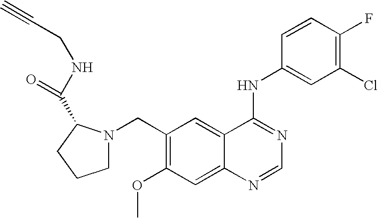

1-({(4-[(3-Chloro-4-fluorophenyl)amino]-7-methoxyquinazolin-6-yl}methyl)-D-proline was coupled with propargylamine using an analogous process to that described in Example 16 to give the title product; ¹H NMR Spectrum: (DMSO d₆) 1.50-1.85 (m, 3H), 2.00-2.20 (m, 1H), 2.35-2.60 (m, 1H+DMSO), 2.80-2.98 (m, 1H), 3.07 (s, 1H), 3.12-3.23 (m, 1H), 3.61 (d, 1H), 3.85-4.1 (m, 3H), 4.02 (s, 3H), 7.23 (s, 1H), 7.44 (dd, 1H), 7.75-7.90 (m, 1H), 8.00-8.11 (m, 1H), 8.11-8.22 (m, 1H), 8.40 (s, 1H), 8.57 (s, 1H), 9.70 (s, 1H); Mass Spectrum: (M+H)⁺ 468.

### EXAMPLE 20

### 1-({4-[(3-Chloro-4-fluorophenyl)amino]-7-methoxyquinazolin-6-yl}methyl)-N-(cyanomethyl)-D-prolinamide

### (Process (c))

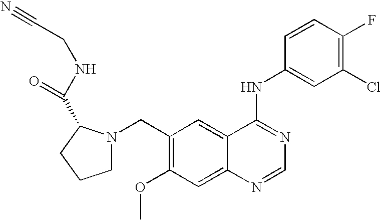

1-({4-[(3-Chloro-4-fluorophenyl)amino]-7-methoxyquinazolin-6-yl}methyl)-D-proline was coupled with aminoacetonitrile using an analogous process to that described in Example 16 to give the title product; ¹H NMR Spectrum: (DMSO d₆) 1.55-1.85 (m, 3H), 2.03-2.25 (m, 1H), 2.35-2.60 (m, 1H+DMSO), 2.85-3.02 (m, 1H), 3.15-3.35 (m, 1H+H₂O), 3.68 (d, 1H), 3.93 (d, 1H), 4.00 (s, 3H), 4.16 (d, 2H), 7.21 (s, 1H), 7.44 (dd, 1H), 7.75-7.90 (m, 1H), 8.10-8.23 (m, 1H), 8.33 (dd, 1H), 8.37 (s, 1H), 8.54 (s, 1H), 9.68 (s, 1H); Mass Spectrum: (M+H)⁺ 469.

### EXAMPLE 21

### 1-({4-[(3-Chloro-4-fluorophenyl)amino]-7-methoxyquinazolin-6-yl}methyl)-N-[2-(dimethylamino)ethyl]-D-prolinamide

### (Process (c))

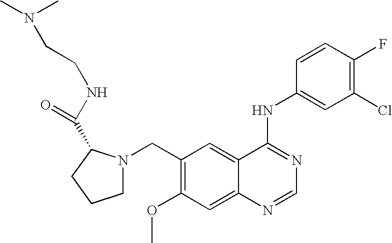

1-({4-[(3-Chloro-4-fluorophenyl)amino]-7-methoxyquinazolin-6-yl}methyl)-D-proline was coupled with N,N-Dimethylethylenediamine using an analogous process to that described in Example 16 to give the title product; ¹H NMR Spectrum: (DMSO d₆+CD₃COOD) 1.60-2.00 (m, 3H+ CHD₂COOD), 2.00-2.20 (m, 1H), 2.30-2.60 (m, 1H+DMSO), 2.65 (s, 6H), 2.85-3.12 (m, 3H), 3.12-3.22 (m, 1H), 3.22-3.35 (m, 1H), 3.35-3.60 (m, 1H), 3.70 (d, 1H), 3.89 (d, 1H), 3.96 (s, 3H), 7.23 (s, 1H), 7.35 (dd, 1H), 7.70-7.90 (m, 1H), 8.10 (dd, 1H), 8.50 (s, 1H), 8.54 (s, 1H); Mass Spectrum: (M+H)⁺ 501.

### EXAMPLE 22

### 1-({4-[(3-Chloro-4-fluorophenyl)amino]-7-methoxyquinazolin-6-yl}methyl)-N-[(5-methylisoxazol-3-yl)methyl]-D-prolinamide

### (Process (c))

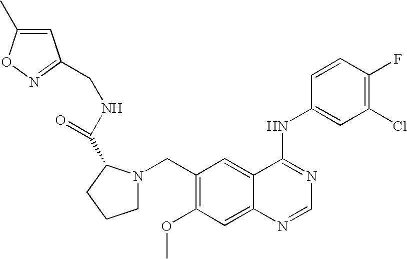

1-({4-[(3-Chloro-4-fluorophenyl)amino]-7-methoxyquinazolin-6-yl}methyl)-D-proline was coupled with N-[(5-methyl-3-isoxazolyl)methyl]amine using an analogous process to that described in Example 16 to give the title product; ¹H NMR Spectrum: (DMSO d₆) 1.55-1.90 (m, 3H), 2.00-2.25 (m, 1H), 2.28 (s, 3H), 2.35-2.65 (m, 1H+DMSO), 2.83-3.02 (m, 1H), 3.10-3.35 (m, 1H+H₂O), 3.66 (d, 1H), 3.90 (s, 3H), 3.96 (d, 1H), 4.30 (d, 2H), 5.91 (s, 1H), 7.17 (s, 1H), 7.43 (dd, 1H), 7.70-7.90 (m, 1H), 8.05-8.20 (m, 1H), 8.20-8.33 (m, 1H), 8.39 (s, 1H), 8.55 (s, 1H), 9.65 (s, 1H); Mass Spectrum: (M+H)⁺ 525.

### EXAMPLE 23

### 2-[(2S)-1-({4-[(3-chloro-2-fluorophenyl)amino]-7-methoxyquinazolin-6-yl}methyl)pyrrolidin-2-yl]acetamide

### (Process (a))

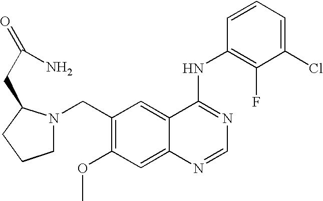

4-[(3-chloro-2-fluorophenyl)amino]-7-methoxyquinazoline-6-carbaldehyde (prepared as described in Example 3) was coupled with 2-[(2S)-pyrrolidin-2-yl]acetamide using an analogous process to that described in the equivalent step in Example 3 to give the title product; ¹H NMR spectrum: (DMSOd₆) 1.53 (m, 1H); 1.67 (m, 2H); 1.95 (m, 1H); 2.21 (m, 2H); 2.47 (m, 1H); 2.82 (m, 1H); 2.95 (m, 1H); 3.42 (d, 1H); 3.95 (s, 3H); 4.10 (d, 1H); 6.79 (brs, 1H); 7.20 (s, 1H); 7.29 (t, 1H); 7.42 (brs, 1H); 7.51 (m, 2H); 8.30 (s, 1H); 8.43 (s, 1H); 9.79 (s, 1H); Mass Spectrum: (M+H)⁺ 444.

The 2-[(2S)-pyrrolidin-2-yl]acetamide starting material was prepared using the same methodology described for the equivalent step in Example 3 from [(2S)-1-(tert-butoxycarbonyl)pyrrolidin-2-yl]acetic acid; ¹H NMR spectrum: (DMSOd₆) 1.22 (m, 1H); 1.62 (m, 2H); 1.76 (m, 1H); 2.13 (dd, 2H); 2.71 (m, 1H); 2.81 (m, 1H); 3.20 (m, 1H); 6.71 (brs, 1H); 7.34 (brs, 1H).

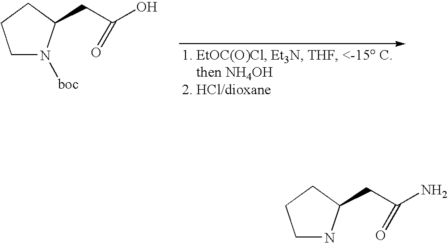

### EXAMPLE 24

### (4R)-3-({4-[(3-Chloro-2-fluorophenyl)amino]-7-methoxyquinazolin-6-yl}methyl)-1,3-thiazolidine-4-carboxamide

### (Process (a))

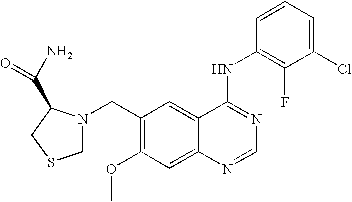

4-[(3-chloro-2-fluorophenyl)amino]-7-methoxyquinazoline-6-carbaldehyde was coupled with (4R)-1,3-thiazolidine-4-carboxamide using an analogous process to that described in the equivalent step in Example 3 to give the title product: ¹H NMR Spectrum: (DMSOd₆) 3.06 (dd, 1H); 3.46 (dd, 1H); 3.79 (d, 1H); 3.84 (d, 1H); 3.97 (s, 3H); 4.08 (m, 3H); 7.24 (s, 1H); 7.30 (t, 1H); 7.40 (brd, 2H); 7.52 (t, 1H); 7.57 (t, 1H); 8.46 (s, 1H); 8.55 (s, 1H); 9.82 (s, 1H). Mass Spectrum: (M+H)⁺ 448.

The (4R)-1,3-thiazolidine-4-carboxamide starting material was prepared using the same methodology as described for the equivalent step in Example 3 from (4R)-3-(tert-butoxycarbonyl)-1,3-thiazolidine-4-carboxylic acid; ¹H NMR Spectrum: (DMSOd₆) 2.85 (dd, 1H); 2.93 (dd, 1H); 3.73 (t, 1H); 4.03 (d, 1H); 4.12 (d, 1H); 7.13 (brs, 1H); 7.44 (brs, 1H).

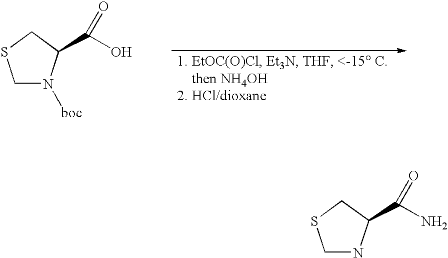

### EXAMPLE 25

### 1-({4-[(3-Chloro-2-fluorophenyl)amino]-7-methoxyquinazolin-6-yl}methyl)-N-methyl-D-prolinamide

### (Process (c))

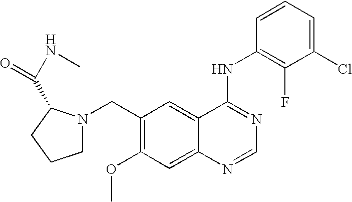

1 ({4-[(3-Chloro-2-fluorophenyl)amino]-7-methoxyquinazolin-6-yl}methyl)-D-proline (150 mg, 0.35 mmol), 1-ethyl-3-(3-dimethylaminopropyl)carbodiimide hydrochloride (100 mg, 0.52 mmol) and 1-hydroxybenztriazole (70 mg, 0.52 mmol) were stirred in N,N-dimethylformamide (5 ml). Triethylamine (170 μl, 1.22 mmol) was added followed by methylamine hydrochloride (28 mg, 0.42 mmol) and the mixture stirred over night at room temperature. The resulting solution was heated to 50° C. and the above quantities of 1-ethyl-3-(3-dimethylaminopropyl)carbodiimide hydrochloride, 1-hydroxybenztriazole, triethylamine and methylamine hydrochloride were again added. After 1 hour the mixture was cooled, diluted with ethyl acetate, washed with brine (×2), dried over magnesium sulfate, filtered and concentrated under reduced pressure. The residues were purified by flash chromatography on SiO₂ eluting with methanol/dichloromethane (2/98) to give the title product as a white foam (100 mg, 65%); ¹H NMR Spectrum: (DMSOd₆) 1.72 (m, 3H); 2.12 (m, 1H); 2.41 (m, 1H); 2.64 (d, 3H); 2.97 (m, 1H); 3.12 (dd, 1H); 3.62 (d, 1H); 3.93 (d, 1H); 4.00 (s, 3H); 7.24 (s, 1H); 7.30 (t, 1H); 7.53 (m, 2H); 7.77 (q, 1H); 8.38 (s, 1H); 8.45 (s, 1H); 9.80 (s, 1H); Mass Spectrum: (M+H)⁺ 444.

The 1-({4-[(3-Chloro-2-fluorophenyl)amino]-7-methoxyquinazolin-6-yl}methyl)-D-proline starting material was prepared as follows:

4-[(3-Chloro-2-fluorophenyl)amino]-7-methoxyquinazoline-6-carbaldehyde was coupled with D-proline using an analogous process to that described in Example 16 to give 1-({4-[(3-chloro-2-fluorophenyl)amino]-7-methoxyquinazolin-6-yl}methyl)-D-proline; ¹H NMR Spectrum: (DMSOd₆) 1.77 (m, 2H); 1.91 (m, 1H); 2.10 (m, 1H); 2.60 (m, 1H); 3.19 (m, 1H); 3.42 (m, 1H); 3.96 (m, 4H); 4.15 (d, 1H); 7.20 (s, 1H); 7.26 (t, 1H); 7.46 (t, 1H); 7.52 (brt, 1H); 8.42 (s, 2H); 10.0 (brs, 1H); Mass Spectrum: (M+H)⁺ 431.

### EXAMPLE 26

### 1-({4-[(3-Chloro-2-fluorophenyl)amino]-7-methoxyquinazolin-6-yl}methyl)-N-ethyl-D-prolinamide

### (Process (c))

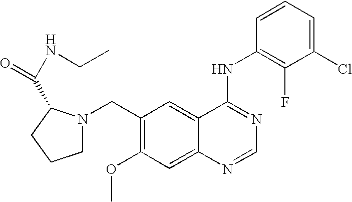

1-({4-[(3-chloro-4-fluorophenyl)amino]-7-methoxyquinazolin-6-yl}methyl)-D-proline was coupled with ethylamine hydrochloride using an analogous process to that described in Example 16 to give the title product; ¹H NMR Spectrum: (DMSOd₆) 0.97 (t, 3H); 1.72 (m, 3H); 2.12 (m, 1H); 2.42 (m, 1H); 2.98 (m, 1H); 3.10 (m, 3H); 3.65 (d, 1H); 3.92 (d, 1H); 3.99 (s, 3H); 7.23 (s, 1H); 7.30 (t, 1H); 7.53 (m, 2H); 7.76 (brt, 1H); 8.38 (s, 1H); 8.45 (s, 1H); 9.81 (s, 1H); Mass Spectrum: (M+H)⁺ 458.

### EXAMPLE 27

### 1-({4-[(3-Chloro-2-fluorophenyl)amino]-7-methoxyquinazolin-6-yl}methyl)-N,N-dimethyl-D-prolinamide

### (Process (c))

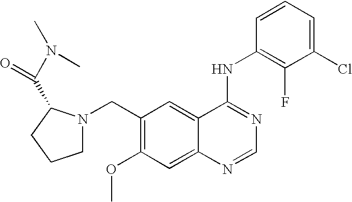

1-({4-[(3-chloro-4-fluorophenyl)amino]-7-methoxyquinazolin-6-yl}methyl)-D-proline was coupled with dimethylamine hydrochloride using an analogous process to that described in Example 16 to give the title product; ¹H NMR Spectrum: (DMSOd₆) 1.75 (m, 3H); 2.09 (m, 1H); 2.45 (q, 1H); 2.77 (s, 3H); 2.97 (s, 3H); 3.07 (m, 1H); 3.55 (m, 1H); 3.77 (d, 1H); 3.86 (d, 1H); 3.93 (s, 3H); 7.19 (s, 1H); 7.28 (t, 1H); 7.51 (m, 2H); 8.28 (s, 1H); 8.43 (s, 1H); 9.86 (s, 1H); Mass Spectrum: (M+H)⁺ 458.

### EXAMPLE 28

### (3S)-1-({4-[(3-Chloro-2-fluorophenyl)amino]-7-methoxyquinazolin-6-yl}methyl)-3-hydroxy-L-prolinamide

### (Process (a))

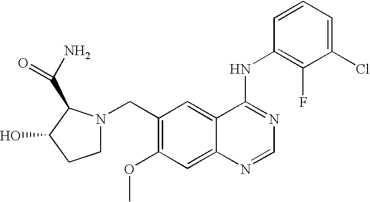

4-[(3-Chloro-2-fluorophenyl)amino]-7-methoxyquinazoline-6-carbaldehyde was coupled with (3S)-3-hydroxy-L-proline using an analogous process to that described in Example 3 to give the title product; ¹H NMR Spectrum: (DMSOd₆) 1.64 (dd, 1H); 1.76 (m, 1H); 2.67 (m, 1H); 2.91 (t, 1H); 3.02 (d, 1H); 3.75 (d, 1H); 3.96 (s, 3H); 4.01 (d, 1H); 4.15 (brs, 1H); 5.11 (d, 1H); 7.19 (d, 1H); 7.22 (s, 1H); 7.30 (t, 1H); 7.46 (d, 1H); 7.50 (t, 1H); 7.56 (t, 1H); 8.40 (s, 1H); 8.45 (s, 1H); 9.77 (s, 1H); Mass Spectrum: (M+H)⁺ 446.

The (3S)-3-hydroxy-L-proline starting material was prepared as follows: (3S)-1-(tert-butoxycarbonyl)-3-hydroxy-1-proline was coupled and deprotected analogously as for the equivalent step in Example 3 to give (3S)-3-hydroxy-L-prolinamide; ¹H NMR Spectrum: (DMSOd₆) 1.57 (m, 2H); 2.90 (m, 3H); 4.14 (m, 1H); 4.84 (brs, 1H); 7.00 (brs, 1H); 7.30 (brs, 1H).

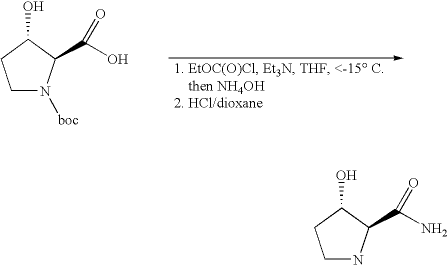

### EXAMPLE 29

### (3R)-1-({4-[(3-chloro-2-fluorophenyl)amino]-7-methoxyquinazolin-6-yl}methyl)pyrrolidine-3-carboxamide

### (Process (a))

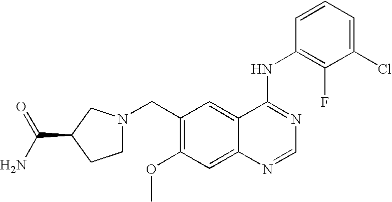

4-[(3-chloro-2-fluorophenyl)amino]-7-methoxyquinazoline-6-carbaldehyde was coupled with (3R)-pyrrolidine-3-carboxamide using an analogous process to that described in Example 3 to give the title product; ¹H NMR Spectrum: (DMSOd₆) 1.93 (q, 2H); 2.50 (m, 2H); 2.75 (m, 1H); 2.87 (m, 2H); 3.71 (d, 1H); 3.76 (d, 1H); 3.96 (s, 3H); 6.74 (brs, 1H); 7.21 (s, 1H); 7.24 (brs, 1H); 7.28 (dt, 1H); 7.51 (m, 2H); 8.35 (s, 1H); 8.43 (s, 1H); 9.83 (s, 1H); Mass Spectrum: (M+H)⁺ 430.

The (3R)-pyrrolidine-3-carboxamide starting material was prepared as follows:

Powdered sodium cyanide (550 mg, 1.3 mmol) was added to a solution of tert-butyl (3S)-3-[(methylsulfonyl)oxy]pyrrolidine-1-carboxylate (2.0 g, 7.54 mmol) in DMSO (10 ml) and the reaction mixture heated at 80° C. for 4 hours. The resulting yellow mixture was cooled and brine (4 ml) and water (4.5 ml) were added. The mixture was extracted with diethyl ether (×3), dried over magnesium sulfate, filtered and concentrated under reduced pressure. The residues were purified by flash chromatography on SiO₂ eluting with diethylether/isohexane (50/50) to give tert-butyl (3R)-3-cyanopyrrolidine-1-carboxylate as a colourless oil (579 mg, 39%); ¹H NMR Spectrum: (DMSOd₆) 1.39 (s, 9H); 2.08 (m, 1H); 2.18 (m, 1H); 3.34 (m, 4H); 3.53 (m, 1H).

tert-Butyl (3R)-3-cyanopyrrolidine-1-carboxylate (575 mg, 2.93 mmol) was dissolved in 4M HCl in dioxane (15 ml) and stirred at room temperature for 2 hours. Water (0.5 ml) was added and the mixture stirred for a further 5 hours, concentrated under reduced pressure and the residue dissolved in methanol. The solution was absorbed onto an Isolute® SCX column, washed with methanol and eluted with 7N ammonia in methanol to give (3R)-pyrrolidine-3-carboxamide as a semi-crystalline solid (285 mg, 85%); ¹H NMR Spectrum: (DMSOd₆ 1.75 (m, 2H); 2.70 (m, 4H); 2.90 (m, 1H); 6.65 (brs, 1H); 7.25 (brs, 1H).

### EXAMPLE 30

### (3S)-1-({4-[(3-Chloro-2-fluorophenyl)amino]-7-methoxyquinazolin-6-yl}methyl)pyrrolidine-3-carboxamide

### (Process (a))

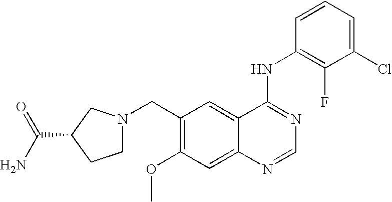

4-[(3-chloro-2-fluorophenyl)amino]-7-methoxyquinazoline-6-carbaldehyde was coupled with (3S)-Pyrrolidine-3-carboxamide using an analogous process to that described in Example 3 to give the title product; ¹H NMR Spectrum: (DMSOd₆) 1.93 (q, 2H); 2.50 (m, 2H); 2.75 (m, 1H); 2.87 (m, 2H); 3.71 (d, 1H); 3.76 (d, 1H); 3.96 (s, 3H); 6.74 (brs, 1H); 7.21 (s, 1H); 7.24 (brs, 1H); 7.28 (dt, 1H); 7.51 (m, 2H); 8.35 (s, 1H); 8.43 (s, 1H); 9.83 (s, 1H); Mass Spectrum: (M+H)⁺ 430.

The (3S)-Pyrrolidine-3-carboxamide starting material was prepared as follows:

tert-butyl (3S)-3-cyanopyrrolidine-1-carboxylate was prepared using the same methodology as described for the equivalent step in the previous example from tert-butyl (3R)-3-[(methylsulfonyl)oxy]pyrrolidine-1-carboxylate. ¹H NMR Spectrum: (DMSOd₆) 1.39 (s, 9H); 2.08 (m, 1H); 2.18 (m, 1H); 3.34 (m, 4H); 3.53 (m, 1H).

(3S)-Pyrrolidine-3-carboxamide was prepared using the same methodology as described for the equivalent step in Example 29 from (3S)-3-cyanopyrrolidine-1-carboxylate. ¹H NMR Spectrum: (DMSOd₆) 1.75 (m, 2H); 2.70 (m, 4H); 2.90 (m, 1H); 6.65 (brs, 1H); 7.25 (brs, 1H).

### EXAMPLE 31

### (4R)-1-({4-[(3-Chloro-2-fluorophenyl)amino]-7-methoxyquinazolin-6-yl}methyl)-4-ethoxy-D-prolinamide

### (Process (a))

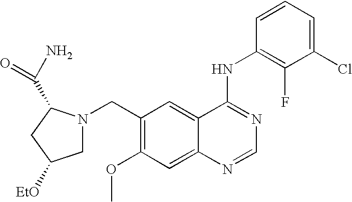

4-[(3-chloro-2-fluorophenyl)amino]-7-methoxyquinazoline-6-carbaldehyde was coupled with (4R)-4-ethoxy-D-prolinamide using an analogous process to that described in Example 3 to give the title product; ¹H NMR Spectrum: (DMSOd₆) 1.03 (t, 3H); 1.79 (m, 1H); 2.41 (m, 1H); 2.54 (d, 1H); 2.96 (d, 1H); 3.08 (t, 1H); 3.31 (m, 2H); 3.58 (d, 1H); 3.96 (m, 5H); 7.14 (d, 1H); 7.23 (s, 1H); 7.30 (t, 1H); 7.36 (d, 1H); 7.50 (t. 1H); 7.57 (t, 1H); 8.38 (s, 1H); 8.45 (s, 1H); 9.80 (s, 1H); Mass Spectrum: (M+H)⁺ 474.

The (4R)-4-ethoxy-D-prolinamide starting material was prepared as follows:

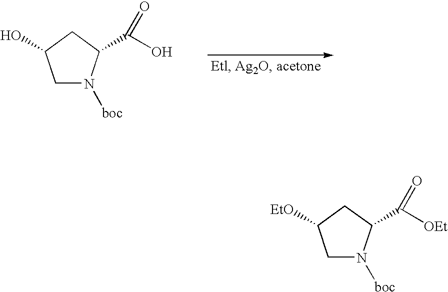

(4R)-1-(tert-butoxycarbonyl)-hydroxy-D-proline was reacted with ethyl iodide under the same conditions described for the equivalent step in Example 10 to give 1-tert-butyl 2-ethyl (2R,4R)-4-ethoxypyrrolidine-1,2-dicarboxylate; ¹H NMR Spectrum: (DMSOd₆, 100° C.) 1.06 (t, 3H); 1.20 (t, 3H); 1.38 (s, 9H); 1.98 (m, 1H); 2.36 (m, 1H); 3.21 (dd, 1H); 3.39 (q, 2H); 3.58 (dd, 1H); 4.09 (m, 3H); 4.21 (dd, 1H).

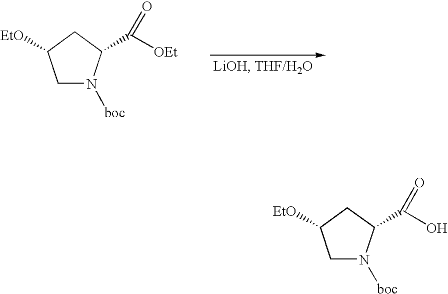

2-ethyl (2R,4R)-4-ethoxypyrrolidine-1,2-dicarboxylate was hydrolysed using the same methodology described for the equivalent step in Example 10 to give (4R)-1-(tert-Butoxycarbonyl)-4-ethoxy-D-proline; ¹H NMR Spectrum: (DMSOd₆, 100° C.) 1.08 (t, 3H); 1.39 (s, 9H); 1.93 (m, 1H); 2.38 (m, 1H); 3.18 (dd, 1H); 3.41 (q, 2H); 3.60 (dd, 1H); 4.04 (dd, 1H); 4.13 (dd, 1H).

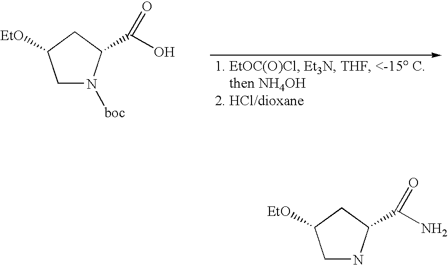

(4R)-1-(tert-Butoxycarbonyl)-4-ethoxy-D-proline was coupled and deprotected using the same methodology as described for the equivalent steps in Example 3 to give (4R)-4-Ethoxy-D-prolinamide; ¹H NMR Spectrum: (DMSOd₆) 1.05 (t, 3H); 1.69 (m, 1H); 2.13 (m, 1H); 2.75 (dd, 1H); 2.86 (m, 2H); 3.33 (q, 2H); 3.39 (dd, 1H); 3.88 (m, 1H); 6.91 (brs, 1H); 7.28 (brs, 1H).

### EXAMPLE 32

### (4S)-1-({4-[(3-Chloro-2-fluorophenyl)amino]-7-methoxyquinazolin-6-yl}methyl)-4-(dimethylamino)-L-prolinamide

### (Process (a))

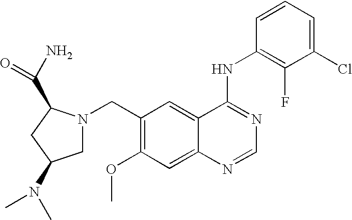

4-[(3-chloro-2-fluorophenyl)amino]-7-methoxyquinazoline-6-carbaldehyde was coupled with (4S)-4-(dimethylamino)-L-prolinamide using an analogous process to that described in Example 3 to give the title product; ¹H NMR Spectrum: (DMSOd₆) 1.66 (m, 1H); 2.06 (s, 6H); 2.26 (m, 1H); 2.54 (m, 1H); 2.81 (m, 1H); 2.92 (dd, 1H); 3.17 (dd, 1H); 3.61 (d, 1H); 3.88 (d, 1H); 3.96 (s, 3H); 7.20 (d, 1H); 7.22 (s, 1H); 7.30 (t, 1H); 7.41 (d, 1H); 7.51 (t, 1H); 7.58 (t, 1H); 8.40 (s, 1H); 8.45 (s, 1H); 9.78 (s, 1H). Mass Spectrum: (M+H)⁺ 473.

The (4S)-4-(dimethylamino)-L-prolinamide starting material was prepared as follows:

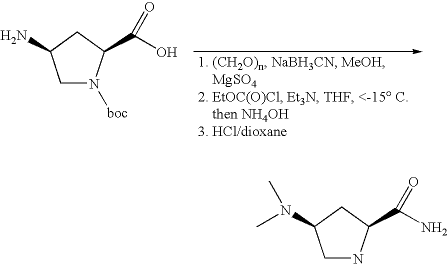

Sodium cyanoborohydride (1.0 g, 17.4 mmol) was added to a stirred suspension of (4S)-4-amino-1-(tert-butoxycarbonyl)-L-proline (1.0 g, 4.34 mmol), magnesium sulfate (1.0 g, 8.69 mmol) and paraformaldehyde (260 mg, 8.68 mmol) in methanol (30 ml). The resulting mixture was heated at 45° C. for 2 hours, cooled, filtered and concentrated under reduced pressure. The crudes were dissolved in methanol, absorbed onto an Isolute® SCX column, washed with methanol and eluted with 7N ammonia in methanol. The filtrates were evaporated to dryness and the residues re-dissolved in tetrahydrofuran (15 ml) and triethylamine (0.59 ml, 4.26 mmol). The resulting mixture was cooled to −15° C. and ethyl chloroformate (0.41 ml, 4.26 mmol) in tetrahydrofuran (3 ml) was slowly added. After 10 minutes, concentrated ammonium hydroxide solution (8 ml) was added and the mixture stirred at 0° C. for 2 hours Saturated ammonium chloride solution was added and the layers partitioned. The aqueous layer was extracted with ethyl acetate and the combined organics dried over magnesium sulfate, filtered and concentrated under reduced pressure. The residues were purified by flash chromatography on SiO₂, eluting with increasingly polar mixtures of methanol/dichloromethane (7.5/92.5-15/85). Fraction containing the desired product were combined and evaporated to give a white solid (200 mg). This was dissolved in 4M HCl in dioxane, stirred for 2 hours and concentrated under reduced pressure. The residue was dissolved in methanol, absorbed onto an Isolute® SCX column, washed with methanol and eluted with 7N ammonia in methanol to give (4S)-4-(dimethylamino)-L-prolinamide as a white solid (93 mg); ¹H NMR Spectrum: (DMSOd₆) 1.41 (m, 1H); 2.09 (s, 6H); 2.16 (m, 1H); 2.44 (m, 2H); 2.96 (m, 1H); 3.49 (t, 1H); 6.93 (brs, 1H); 7.31 (brs, 1H).

### EXAMPLE 33

### (2S,4S)-1-({4-[(3-Chloro-2-fluorophenyl)amino]-7-methoxyquinazolin-6-yl}methyl)-4-hydroxypiperidine-2-carboxamide

### (Process (a))

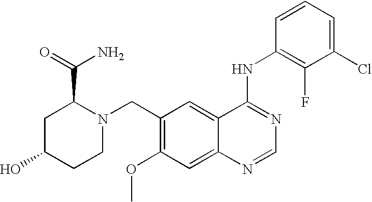

4-[(3-Chloro-2-fluorophenyl)amino]-7-methoxyquinazoline-6-carbaldehyde was coupled with (2S,4S)-4-hydroxypiperidine-2-carboxamide using an analogous process to that described in Example 3 to give the title product; ¹H NMR Spectrum: (DMSOd₆) 1.51 (m, 1H); 1.63 (m, 1H); 1.74 (m, 1H); 1.86 (m, 1H); 2.47 (m, 1H); 2.70 (m, 1H); 3.16 (dd, 1H); 3.54 (d, 1H); 3.79 (m, 2H); 3.96 (s, 3H); 4.61 (d, 1H); 7.14 (d, 1H); 7.21 (s, 1H); 7.27 (d, 1H); 7.30 (t, 1H); 7.51 (t, 1H); 7.58 (t, 1H); 8.42 (s, 1H); 8.44 (s, 1H); 9.77 (s, 1H); Mass Spectrum: M+H)⁺ 460.

The (2S,4S)-4-hydroxypiperidine-2-carboxamide starting material was prepared as follows:

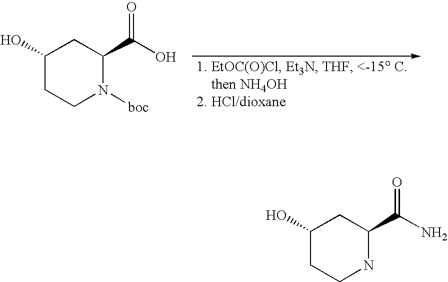

(2S,4S)-1-(tert-butoxycarbonyl)-4-hydroxypiperidine-2-carboxylic acid was coupled and deprotected using the same methodology described for the equivalent steps in Example 3 to give (2S,4S)-4-hydroxypiperidine-2-carboxamide; ¹H NMR Spectrum: (DMSOd₆) 1.32 (m, 1H); 1.52 (m, 2H); 1.67 (m, 1H); 2.61 (m, 1H); 2.75 (brs, 1H); 2.83 (m, 1H); 3.37 (dd, 1H); 3.76 (m, 1H); 4.48 (s, 1H); 6.88 (brs, 1H); 7.13 (brs, 1H).

### EXAMPLE 34

### 1-({4-[(3-Chloro-2-fluorophenyl)amino]-7-methoxyquinazolin-6-yl}methyl)-5-methyl-L-Prolinamide

### (Process (c))

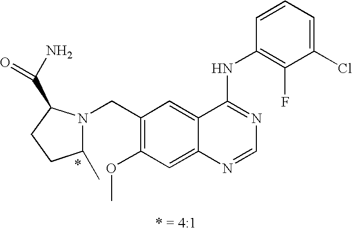

1-({4-[(3-Chloro-2-fluorophenyl)amino]-7-methoxyquinazolin-6-yl}methyl)-5-methyl-L-proline (585 mg, 1.31 mmol as a 4:1 mixture of the 5-methyl isomers) and triethylamine (0.202 ml, 1.45 mmol) in tetrahydrofuran (5 ml) were cooled to −15° C. and ethyl chloroformate (0.138 ml, 1.45 mmol) in tetrahydrofuran (3 ml) was added drop wise. After 10 minutes, concentrated ammonium hydroxide solution (3 ml) was added and the mixture stirred at 0° C. for 2 hours. Saturated ammonium chloride solution was added and the layers partitioned. The aqueous layer was extracted with ethyl acetate and the combined organics were dried over magnesium sulfate, filtered and evaporated. The residues were purified by flash chromatography on SiO₂ eluting with methanol/dichloromethane (3/97) to give title product as a 4:1 mixture of the 5-methyl isomers (340 mg, 58%); ¹H NMR Spectrum: (DMSOd₆) 0.95* (d, 3H); 1.08 (d, 3H); 1.34 (m, 1H); 1.45* (m, 1H); 1.72 (m, 1H); 1.88 (m, 1H); 2.01 (m, 1H); 2.26* (m, 1H); 2.88 (m, 1H); 3.18 (dd, 1H); 3.31* (m, 1H); 3.73 (m, 1H); 3.96 (m, 4H); 6.92 (d, 1H); 7.02* (d, 1H); 7.19-7.36 (m, 3H); 7.49 (m, 1H); 7.57 (m, 1H); 8.36-8.44 (m, 2H); 9.74 (m, 1H) (*=minor isomer peaks); Mass Spectrum: (M+H)⁺ 444.

The 1-({4-[(3-Chloro-2-fluorophenyl)amino]-7-methoxyquinazolin-6-yl}methyl)-5-methyl-L-proline starting material was prepared as follows:

4-[(3-Chloro-2-fluorophenyl)amino]-7-methoxyquinazoline-6-carbaldehyde was coupled with 5-methyl-L-proline using the same methodology described for the equivalent step in Example 3 to give 1-({4-[(3-chloro-2-fluorophenyl)amino]-7-methoxyquinazolin-6-yl}methyl)-5-methyl-L-proline as a 4:1 mixture of the 5-methyl isomers; ¹H NMR Spectrum: (DMSOd₆+D₂O) 1.11* (d, 3H); 1.33 (d, 3H); 1.48 (m, 1H); 1.51* (m, 1H); 1.78* (m, 1H); 2.04 (m, 1H); 2.18 (m, 1H); 3.41 (m, 1H); 3.66 (m, 1H); 3.94-4.17 (m, 4H); 4.49 )d, 1H); 7.26 (m, 1H); 7.52 (m, 2H); 8.42 (m, 2H) (*=minor isomer peaks); Mass Spectrum: (M+H)⁺ 445.

### EXAMPLE 35

### 1-({4-[(3-Chloro-2-fluorophenyl)amino]-7-methoxyquinazolin-6-yl}methyl)piperazine-2-carboxamide

### (Process (a))

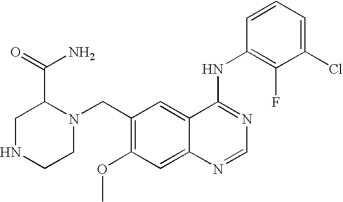

A solution of tert-butyl 3-(aminocarbonyl)-4-({4-[(3-chloro-2-fluorophenyl)amino]-7-methoxyquinazolin-6-yl}methyl)piperazine-1-carboxylate (400 mg, 0.73 mmol) in 4M HCl in dioxane (20 ml) was stirred for 3 hours. The reaction mixture was then evaporated and the residue was re-dissolved in methanol. This was absorbed onto an Isolute® SCX column, washed with methanol and eluted with 7N ammonia in methanol to give the title product as a white solid (325 mg, 100%); ¹H NMR Spectrum: (DMSOd₆) 2.04 (m, 1H); 2.40 (brs, 1H); 2.73 (m, 5H); 3.01 (d, 1H); 3.41 (d, 1H); 3.79 (d, 1H); 3.96 (s, 3H); 7.23 (m, 3H); 7.30 (t, 1H); 7.51 (t, 1H); 7.59 (t, 1H); 8.41 (s, 1H); 8.44 (s, 1H); 9.74 (s, 1H); Mass Spectrum: (M+H)⁺ 445.

The tert-butyl 3-(aminocarbonyl)-4-({4-[(3-chloro-2-fluorophenyl)amino]-7-methoxyquinazolin-6-yl}methyl)piperazine-1-carboxylate starting material was prepared as follows:

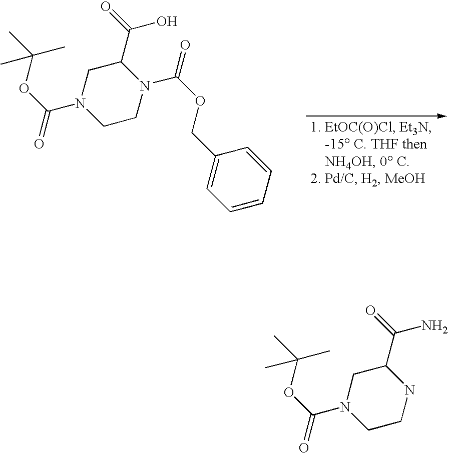

1-[(Benzyloxy)carbonyl]-4-(tert-butoxycarbonyl)piperazine-2-carboxylic acid (2.0 g, 5.49 mmol) and triethylamine (0.842 ml, 6.04 mmol) in tetrahydrofuran (20 ml) were cooled to −15° C. and ethyl chloroformate (0.577 ml, 6.04 mmol) in tetrahydrofuran (5 ml) was added dropwise. After 10 minutes, concentrated ammonium hydroxide solution (10 ml) was added and the mixture stirred at 0° C. for 2 hours. Saturated ammonium chloride solution was then added and the layers partitioned. The aqueous layer was extracted with ethyl acetate and the combined organics dried over magnesium sulfate, filtered and evaporated. The residue was re-dissolved in methanol (50 ml) and the system purged with nitrogen. 10% palladium on carbon (0.18 g, 10% by mass of residue) was added and the mixture stirred under a hydrogen atmosphere for 3 hours. The reaction mixture was filtered and concentrated under reduced pressure to give tert-butyl 3-(aminocarbonyl)piperazine-1-carboxylate as a white solid (700 mg, 56%); ¹H NMR Spectrum: (DMSOd₆) 1.40 (s, 9H); 2.54 (d, 1H); 2.83 (m, 3H); 3.05 (dd, 1H); 3.62 (d, 1H); 3.85 (m, 1H); 7.08 (brs, 1H); 7.24 (brs, 1H).

4-[(3-chloro-2-fluorophenyl)amino]-7-methoxyquinazoline-6-carbaldehyde was coupled with tert-butyl 3-(aminocarbonyl)piperazine-1-carboxylate using the same methodology described for the equivalent step in Example 3 to give tert-butyl 3-(aminocarbonyl)-4-({4-[(3-chloro-2-fluorophenyl)amino]-7-methoxyquinazolin-6-yl}methyl)piperazine-1-carboxylate; ¹H NMR Spectrum: (DMSOd₆) 1.41 (s, 9H); 2.13 (t, 1H); 2.90 (dd, 2H); 3.08 (t, 1H); 3.18 (m, 1H); 3.49 (d, 1H); 3.65 (d, 1H); 3.86 (d, 2H); 3.95 (s, 3H); 7.22 (s, 1H); 7.32 (m, 3H); 7.51 (t, 1H); 7.58 (t, 1H); 8.39 (s, 1H); 8.45 (s, 1H); 9.74 (s, 1H); Mass Spectrum: (M+H)+545.

### EXAMPLE 36

### 1-({4-[(3-Chloro-2-fluorophenyl)amino]-7-methoxyquinazolin-6-yl}methyl)-4-methylipiperazine-2-carboxamide

### (Process (d))

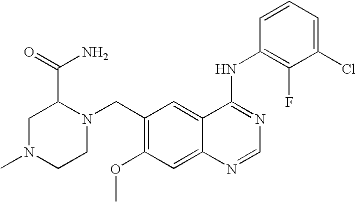

Magnesium sulfate (73 mg, 0.61 mmol), paraformaldehyde (76 mg, 0.61 mmol) and sodium cyanoborohydride (18 mg, 1.21 mmol) were added to a solution of 1-({4-[(3-chloro-2-fluorophenyl)amino]-7-methoxyquinazolin-6-yl}methyl)piperazine-2-carboxamide (135 mg, 0.30 mmol, Example 35) in methanol (5 ml). The mixture was heated at 50° C. for 1.5 hours, cooled, filtered, absorbed onto an Isolute® SCX column, washed with methanol and eluted with 7N ammonia in methanol. Filtrates were combined and evaporated. The residues were purified by flash chromatography on SiO₂, eluting with increasingly polar mixtures of methanol/dichloromethane (5/95-10/90) to give the title product as a white solid (105 mg, 76%); ¹H NMR Spectrum: (DMSOd₆) 2.18 (m, 6H); 2.57 (d, 1H); 2.79 (m, 2H); 2.92 (dd, 1H); 3.44 (d, 1H); 3.84 (d, 1H); 3.96 (s, 3H); 7.22 (s, 1H); 7.30 (m, 3H); 7.51 (t, 1H); 7.58 (t, 1H); 8.40 (s, 1H); 8.44 (s, 1H); 9.75 (s, 1H); Mass Spectrum: (M+Et)⁺ 459.

### EXAMPLE 37

### 1-({4-[(3-Chloro-2-fluorophenyl)amino]-7-methoxyquinazolin-6-yl}methyl)-4-(2-methoxyethyl)piperazine-2-carboxamide

### (Process (d))

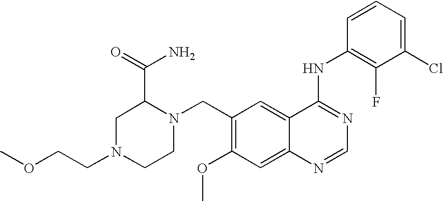

Sodium triacetoxyborohydride (79 mg, 0.37 mmol) was added to a stirred suspension of 1-({4-[(3-chloro-2-fluorophenyl)amino]-7-methoxyquinazolin-6-yl}methyl)piperazine-2-carboxamide (110 mg, 0.25 mmol, Example 35), methoxyacetaldehyde (24 mg of a solution containing 17% water, 0.37 mmol) and 3A molecular sieves (250 mg) in 5% acetic acid/dichloromethane (10 ml). After 1 hour no product was observed so a further 10 equivalents of methoxyacetaldehyde and sodium triacetoxyborohydride were added. After 2 hours the mixture was filtered, concentrated under reduced pressure and re-dissolved in methanol. This was absorbed onto an Isolute® SCX column and eluted with methanol followed by 7N ammonia in methanol. Fractions containing the desired product were combined and evaporated. The residues were purified by flash chromatography on SiO₂, eluting with methanol/dichloromethane (5/95) to give the title product as a white solid (30 mg, 24%); ¹H NMR Spectrum: (DMSOd₆+d₄ AcOH) 2.30-2.41 (m, 3H); 2.66 (m, 2H); 2.82 (d, 2H); 3.04 (m, 2H); 3.24 (s, 3H); 3.49 (m, 3H); 3.84 (d, 1H); 3.95 (s, 3H); 7.22 (s, 1H); 7.28 (t, 1H); 7.48 (t, 1H); 7.56 (t, 1H); 8.38 (s, 1H); 8.43 (s, 1H); Mass Spectrum: (M+H)⁺ 503.

### EXAMPLE 38

### 1-({4-[(3-Chloro-2-fluorophenyl)amino]-7-methoxyquinazolin-6-yl}methyl)-4-(dimethylamino)piperidine-4-carboxamide

### (Process (a))

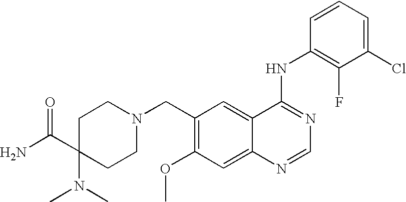

4-[(3-chloro-2-fluorophenyl)amino]-7-methoxyquinazoline-6-carbaldehyde was coupled with 4-(dimethylamino)piperidine-4-carboxamide (commercially available or can be prepared by debenzylation of 1-benzyl-4-(dimethylamino)piperidine-4-carboxamide as described in JP 03188030 Example 2 therein) using the same methodology described for the equivalent step in Example 3 to give the title product; ¹H NMR Spectrum: (DMSOd₆) 1.65 (t, 2H); 2.03 (d, 2H); 2.16 (m, 8H); 2.69 (m, 2H); 3.58 (s, 2H); 3.95 (s, 3H); 6.96 (d, 2H); 7.20 (s, 1H); 7.28 (t, 1H); 7.49 (t, 1H); 7.54 (t, 1H); 8.32 (s, 1H); 8.43 (s, 1H); 9.83 (s, 1H); Mass Spectrum: 487.

### EXAMPLE 39

### 1-({4-[(3-Chloro-2-fluorophenyl)amino]-7-methoxyquinazolin-6-yl}methyl)-2-methylprolinamide

(process (a))

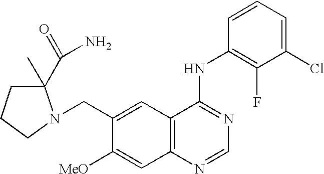

4-[(3-Chloro-2-fluorophenyl)amino]-7-methoxyquinazoline-6-carbaldehyde was coupled with 2-methylprolinamide using the same methodology described for the equivalent step in Example 3 to give the title product; ¹H NMR Spectrum: (DMSOd₆) 1.22 (s, 3H); 1.73 (m, 3H); 2.06 (m, 3H); 2.49 (s, 1H); 2.87 (m, 1H); 3.48 (d, 1H); 3.86 (d, 1H); 3.95 (s, 3H); 7.12 (s, 1H); 7.21 (s, 1H); 7.28 (t, 1H); 7.52 (m, 2H); 7.61 (s, 1H); 8.37 (s, 1H); 8.42 (s, 1H); 9.77 (s, 1H); Mass Spectrum: (M+H)⁺ 444.

The 2-methylprolinamide starting material was prepared as follows:

1-(tert-butoxycarbonyl)-2-methylproline was coupled and deprotected using the same methodology described for the equivalent step in Example 3 to give 2-methylprolinamide; ¹H NMR (spectrum): (DMSOd₆) 1.22 (s, 3H); 1.40 (m, 1H); 1.58 (m, 2H); 2.02 (m, 1H); 2.70 (m, 1H); 2.92 (m, 1H); 6.86 (s, 1H); 7.41 (s, 1H).

### EXAMPLE 40

### (3S)-1-({4-[(3-Chloro-2-fluorophenyl)amino]-7-methoxyquinazolin-6-yl}methyl)-3-methyl-L-prolinamide

### (Process (c)

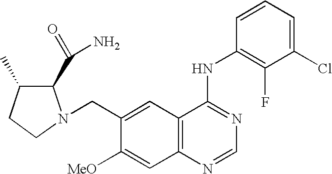

(3S)-1-({4-[(3-chloro-2-fluorophenyl)amino]-7-methoxyquinazolin-6-yl}methyl)-3-methyl-L-proline was coupled using the same methodology described for the equivalent step in Example 34 to give the title product; ¹H NMR Spectrum: (DMSOd₆) 1.09 (d, 3H); 1.93 (m, 1H); 2.12 (m, 1H); 2.40 (m, 1H); 2.49 (m, 1H); 2.59 (d, 1H); 2.96 (m, 1H); 3.56 (d, 1H); 3.92 (m, 4H); 7.15 (s, 1H); 7.19 (s, 1H); 7.28 (m, 2H); 7.52 (m, 2H); 8.37 (s, 1H); 8.42 (s, 1H); 9.76 (s, 1H); Mass Spectrum: (M+H)⁺ 444.

The (3S)-1-({4-[(3-chloro-2-fluorophenyl)amino]-7-methoxyquinazolin-6-yl}methyl)-3-methyl-L-proline starting material was prepared as follows:

4-[(3-chloro-2-fluorophenyl)amino]-7-methoxyquinazoline-6-carbaldehyde was coupled with (3S)-3-methyl-L-proline using the same methodology described for the equivalent step in Example 3 to give (3S)-1-({4-[(3-chloro-2-fluorophenyl)amino]-7-methoxyquinazolin-6-yl}methyl)-3-methyl-L-proline; ¹H NMR Spectrum: (DMSOd₆) 1.08 (d, 3H); 1.41 (m, 1H); 1.99 (m, 1H); 2.25 (m, 1H); 2.63 (q, 1H); 2.92 (d, 1H); 3.14 (m, 1H); 3.91 (m, 4H); 4.06 (d, 1H); 7.18 (s, 1H); 7.25 (m, 1H); 7.48 (m, 2H); 8.39 (m, 2H); Mass Spectrum: (M+H)⁺ 445.

### EXAMPLE 41

### 1-({4-[(3-Chloro-2-fluorophenyl)amino]-7-methoxyquinazolin-6-yl}methyl)azetidine-3-carboxamide

### (Process (c)

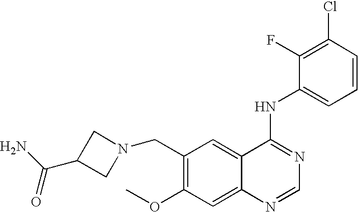

Diisopropylethylamine (0.42 ml, 2.40 mmol) then HATU (274 mg, 0.72 mmol) were added to a solution of 1-({4-[(3-chloro-2-fluorophenyl)amino]-7-methoxyquinazolin-6-yl}methyl)azetidine-3-carboxylic acid (200 mg, 0.48 mmol) in DMF (2 ml). After 10 minutes, ammonium chloride (39 mg, 0.72 mmol) was added and the mixture stirred overnight at room temperature. The crude product was purified using mass triggered preparative HPLC to give the title product as a powder (11 mg, 5%); ¹H NMR Spectrum: (DMSO d₆) 3.16 (m, 1H), 3.22 (brs, 2H), 3.60 (brs, 2H), 3.81 (brs, 2H), 3.96 (s, 3H), 6.94 (brs, 1H), 7.20 (s, 1H), 7.28 (t, 1H), 7.36 (brs, 1H), 7.48-7.53 (m, 2H), 8.28 (s, 1H), 8.43 (s, 1H)+NH; Mass Spectrum: (M+H)⁺ 416.

The 1-({4-[(3-chloro-2-fluorophenyl)amino]-7-methoxyquinazolin-6-yl}methyl)azetidine-3-carboxylic acid starting material was prepared as follows.

4-[(3-chloro-2-fluorophenyl)amino]-7-methoxyquinazoline-6-carbaldehyde was coupled with 3-carboxylazetidine using the same methodology described for the equivalent step in Example 3 to give 1-({4-[(3-chloro-2-fluorophenyl)amino]-7-methoxyquinazolin-6-yl}methyl)azetidine-3-carboxylic acid as a white powder (120 mg, 65%); ¹H NMR Spectrum: (DMSO d₆) 3.17 (m, 1H), 3.26 (m, 2H), 3.52 (m, 2H), 3.67 (s, 2H), 3.94 (s, 3H), 7.17 (s, 1H), 7.25 (t, 1H), 7.46 (m, 2H), 8.24 (s, 1H), 8.41 (s, 1H); Mass Spectrum: (M+H)⁺ 417.

### EXAMPLE 42

### 1-({4-[(3-Chloro-2-fluorophenyl)amino]-7-methoxyquinazolin-6-yl}methyl)azetidine-2-carboxamide

### (Process (c)

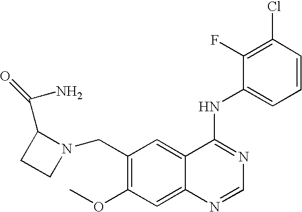

1-({4-[(3-Chloro-2-fluorophenyl)amino]-7-methoxyquinazolin-6-yl}methyl)azetidine-2-carboxylic acid (200 mg, 0.48 mmol) and triethylamine (74 ul, 0.53 mmol) in tetrahydrofuran (9 ml) were cooled to −15° C. Ethyl chloroformate (51 ul, 0.53 mmol) was added dropwise followed after 10 minutes by concentrated ammonium hydroxide (0.84 ml). The mixture was stirred at 0° C. for 2 hours. Saturated ammonium chloride solution was added and the layers separated. The aqueous layer was extracted with ethyl acetate and the combined organics dried over magnesium sulfate, filtered and concentrated under reduced pressure. The crude product was purified by mass triggered preparative HPLC to give the title product as a powder (28 mg, 14%); ¹H NMR Spectrum: (CDCl₃) 2.01 (m, 1H), 2.26 (m, 1H), 2.92 (m, 1H), 3.31 (signal hidden under solvent, 1H), 3.60-3.67 (m, 2H), 3.80 (d, 1H), 3.95 (s, 3H), 7.20-7.30 (m, 4H), 7.48-7.55 (m, 2H), 8.32 (s, 1H), 8.43 (s, 1H), 9.82 (brs, 1H); Mass Spectrum: (M+H)⁺ 416.

The 1-({4-[(3-Chloro-2-fluorophenyl)amino]-7-methoxyquinazolin-6-yl}methyl)azetidine-2-carboxylic acid starting material was prepared as follows:

4-[(3-Chloro-2-fluorophenyl)amino]-7-methoxyquinazoline-6-carbaldehyde was coupled with azetidine-2-carboxylic acid using the same methodology described for the equivalent step in Example 3 to give 1-({4-[(3-chloro-2-fluorophenyl)amino]-7-methoxyquinazolin-6-yl}methyl)azetidine-2-carboxylic acid; ¹H NMR Spectrum: (DMSO d₆) 2.14 (m, 2H), 2.85 (m, 1H), 3.59 (t, 1H), 3.72 (d, 1H), 3.79 (s, 1H), 3.87 (d, 1H), 3.93 (s, 3H), 7.14 (s, 1H), 7.25 (t, 1H), 7.45 (t, 1H), 7.53 (t, 1H), 8.40 (s, 1H), 8.65 (s, 1H); Mass Spectrum: (M+H)⁺ 417.

### EXAMPLE 43

### 1-(1-{4-[(3-Chloro-2-fluorophenyl)amino]-7-methoxyquinazolin-6-yl}ethyl)-L-prolinamide

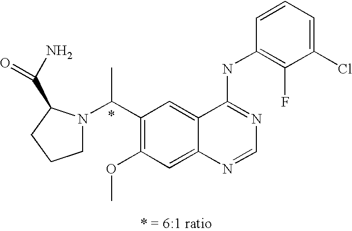

1-{4-[(3-Chloro-2-fluorophenyl)amino]-7-methoxyquinazolin-6-yl}ethanone was coupled with (2S)-prolinamide analogously using an analogous process to that described in Example 3 to give the title product as a 6:1 mixture of isomers; ¹H NMR (spectrum): (DMSOd₆+D₂O) 1.31 (d, 3H); 1.44* (d, 3H); 1.72 (m, 3H); 2.08 (m, 1H); 2.21 m, 1H); 2.79* (m, 1H); 2.98 (m, 1H); 3.21 (m, 1H); 3.95 (s, 3H); 3.96* (s, 3H); 4.20 (m, 1H); 4.39* (m, 1H); 7.19* (s, 1H); 7.20 (s, 1H); 7.31 (dt, 1H); 7.55 (m, 2H); 8.41 (s, 1H); 8.42* (s, 1H); 8.43* (s, 1H); 8.46 (s, 1H) (*=minor isomer peaks); Mass spectrum: (M−H)⁻ 442.

The 1-{4-[(3-chloro-2-fluorophenyl)amino]-7-methoxyquinazolin-6-yl}ethanone used as starting material was prepared as follows:

4-[(3-Chloro-2-fluorophenyl)amino]-7-methoxyquinazolin-6-yl trifluoromethanesulfonate (3 g, 6.64 mmol, Example 1, preparation of starting materials) was dissolved in DMF (21 ml) and n-butyl vinyl ether (4.3 ml, 33.2 mmol), triethylamine (2.3 ml, 16.6 mmol), 1,3-bis(diphenylphosphino)propane (438 mg, 1.06 mmol) and palladium acetate (223 mg, 1 mmol) were added. The mixture was heated at 80° C. for 2 hours, then cooled to room temperature and stirred over night. 2M Hydrochloric acid (24 ml) was added and the mixture stirred for 0.5 hours. The mixture was basified with saturated, aqueous sodium hydrogen carbonate and extracted with ethyl acetate. The organic extracts were washed with brine, dried (MgSO₄) and concentrated under reduced pressure. The resulting solid was suspended in methanol and filtered to give 1-{4-[(3-chloro-2-fluorophenyl)amino]-7-methoxyquinazolin-6-yl}ethanone (1.7 g, 74%) as a pale yellow solid; ¹H NMR (spectrum): (DMSOd₆) 2.62 (s, 3H); 4.02 (s, 3H); 7.27 (m, 2H); 7.49 (t, 2H); 8.48 (s, 1H); 8.72 (s, 1H); 10.19 (s, 1H); Mass Spectrum: (MH)⁺ 346.

### EXAMPLE 44

### (1S,5R)-3-({4-[(3-Chloro-2-fluorophenyl)amino]-7-methoxyquinazolin-6-yl}methyl)-3-azabicyclo[3.1.0]hexane-2-carboxamide

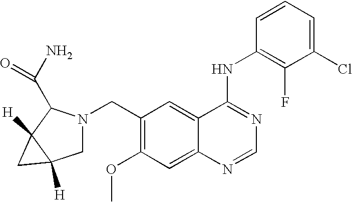

(1S,5R)-3-({4-[(3-Chloro-2-fluorophenyl)amino]-7-methoxyquinazolin-6-yl}methyl)-3-azabicyclo[3.1.0]hexane-2-carboxamide was synthesised using the same methodology as described for the equivalent step in Example 41 from (1S,5R)-3-({4-[(3-chloro-2-fluorophenyl)amino]-7-methoxyquinazolin-6-yl}methyl)-3-azabicyclo[3.1.0]hexane-2-carboxylic acid; ¹H NMR Spectrum: (DMSO d₆) 0.34 (m, 1H); 0.78 (q, 1H); 1.45 (m, 1H); 1.67 (m, 1H); 2.52 (m, 1H); 2.87 (d, 1H); 3.15 (d, 1H); 3.50 (d, 1H); 3.88 (d, 1H); 3.95 (s, 1H); 7.15 (d, 1H); 7.22 (s, 1H); 7.29 (m, 2H); 7.51 (m, 1H); 7.58 (m, 1H); 8.29 (s, 1H); 8.45 (s, 1H); 9.75 (s, 1H); Mass Spectrum: (M+H)⁺ 442.

The (1S,5R)-3-({4-[(3-chloro-2-fluorophenyl)amino]-7-methoxyquinazolin-6-yl}methyl)-3-azabicyclo[3.1.0]hexane-2-carboxylic acid used as the starting material was prepared as follows:

4-[(3-chloro-2-fluorophenyl)amino]-7-methoxyquinazoline-6-carbaldehyde was coupled with cis-3-azabicyclo[3.1.0]hexane-2-carboxylic acid (Aldrich) using the same methodology as described for the equivalent step in Example 3 to give (1S,5R)-3-({4-[(3-chloro-2-fluorophenyl)amino]-7-methoxyquinazolin-6-yl}methyl)-3-azabicyclo[3.1.0]hexane-2-carboxylic acid. ¹H NMR Spectrum: (DMSO d₆) 0.30 (m, 1H); 0.89 (q, 1H); 1.37 (m, 1H); 1.63 (m, 1H); 2.59 (dd, 1H); 3.02 (d, 1H); 3.24 (d, 1H); 3.79 (d, 1H); 3.91 (m, 4H); 7.19 (s, 1H); 7.27 (t, 1H); 7.48 (t, 1H); 7.54 (t, 1H); 8.24 (s, 1H); 8.43 (s, 1H); 9.87 (brs, 1H); Mass Spectrum: (M+H)⁺ 443.

### EXAMPLE 45

(1R,5S,6r)-3-({4-[(3-chloro-2-fluorophenyl)amino]-7-methoxyquinazolin-6-yl}methyl)-3-azabicyclo[3.1.0]hexane-6-carboxamide

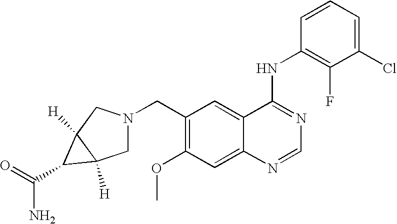

4-[(3-chloro-2-fluorophenyl)amino]-7-methoxyquinazoline-6-carbaldehyde was coupled with (1R,5S,6r)-3-azabicyclo[3.1.0]hexane-6-carboxamide using the same methodology as described for the equivalent step in Example 3 to give (1R,5S)-3-({4-[(3-chloro-2-fluorophenyl)amino]-7-methoxyquinazolin-6-yl}methyl)-3-azabicyclo[3.1.0]hexane-6-carboxamide; ¹H NMR Spectrum: DMSO d₆) 1.72 (m, 2H); 1.91 (m, 1H); 2.52 (m, 2H); 3.00 (d, 2H); 3.75 (s, 2H); 3.96 (s, 3H); 6.66 (s, 1H); 7.21 (s, 1H); 7.29 (t, 1H); 7.37 (s, 1H); 7.52 (m, 2H); 8.24 (s, 1H); 8.43 (s, 1H); 9.79 (s, 1H). Mass Spectrum: (MH)⁺ 442.

The (1R,5S,6r)-3-azabicyclo[3.1.0]hexane-6-carboxamide used as starting material was prepared as follows:

(1R,5S)-3-[(Benzyloxy)carbonyl]-3-azabicyclo[3.1.0]hexane-6-carboxylic acid was coupled and deprotected using the same methodology as described for the equivalent steps in Example 35 to give (1R,5S,6r)-3-azabicyclo[3.1.0]hexane-6-carboxamide; ¹H NMR Spectrum: (DMSO d₆) 1.39 (m, 1H); 1.65 (m, 2H); 2.72 (d, 2H); 2.87 (d, 2H); 6.65 (brs, 1H); 7.25 (brs, 1H).

### EXAMPLE 46

### Pharmaceutical Compositions

The following illustrates representative pharmaceutical dosage forms of the invention as defined herein (the active ingredient being termed “Compound X”) which may be prepared, for therapeutic or prophylactic use in humans:

| (a) | Tablet I | mg/tablet |
|---|---|---|
| | Compound X | 100 |
| | Lactose Ph. Eur | 182.75 |
| | Croscarmellose sodium | 12.0 |
| | Maize starch paste (5% w/v paste) | 2.25 |
| | Magnesium stearate | 3.0 |

| (b) | Injection I | (50 mg/ml) | |
|---|---|---|---|
| | Compound X | 5.0% | w/v |
| | 1M Sodium hydroxide solution | 15.0% | v/v |
| | 0.1M Hydrochloric acid (to adjust pH to 7.6) | 4.5% | w/v |
| | Polyethylene glycol 400 | | |
| | Water for injection to 100%. | | |

The above compositions may be prepared by conventional procedures well known in the pharmaceutical art. For example, Tablet I may be prepared by blending the components together and compressing the mixture into a tablet.

1. A quinazoline derivative of the Formula I: 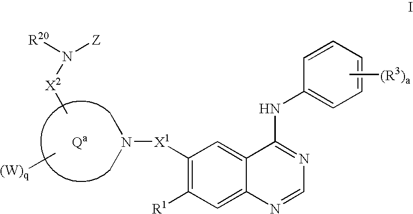
wherein:
R¹ is selected from hydrogen, hydroxy, (1-6C)alkoxy, (2-6C)alkenyloxy, (2-6C)alkynyloxy, or a group of the formula:
Q¹-X³—
wherein X³ is O or S, and Q¹ is (3-7C)cycloalkyl, (3-7C)cycloalkyl-(1-6C)alkyl, (3-7C)cycloalkenyl, (3-7C)cycloalkenyl-(1-6C)alkyl, heterocyclyl or heterocyclyl-(1-6C)alkyl,
and wherein adjacent carbon atoms in any (2-6C)alkylene chain within a R¹ substituent are optionally separated by the insertion into the chain of a group selected from O, S, SO, SO₂, N(R⁴), CO, CH(OR⁴), CON(R⁴), N(R⁴)CO, SO₂N(R⁴), N(R⁴)SO₂, CH═CH and C≡C wherein R⁴ is hydrogen or (1-6C)alkyl,
and wherein any CH₂═CH— or HC≡C— group within a R¹ substituent optionally bears at the terminal CH₂═ or HC≡ position a substituent selected from halogeno, carboxy, carbamoyl, (1-6C)alkoxycarbonyl, N-(1-6C)alkylcarbamoyl, N,N-di-[(1-6C)alkyl]carbamoyl, amino-(1-6C)alkyl, (1-6C)alkylamino-(1-6C)alkyl and di-[(1-6C)alkyl]amino-(1-6C)alkyl or from a group of the formula:
Q²-X⁴—
wherein X⁴ is a direct bond or is selected from CO and N(R⁵)CO, wherein R⁵ is hydrogen or (1-6C)alkyl, and Q² is heterocyclyl or heterocyclyl-(1-6C)alkyl,
and wherein any alkyl or alkylene group within a R¹ substituent optionally bears one or more halogeno, (1-6C)alkyl, hydroxy, cyano, amino, carboxy, carbamoyl, sulfamoyl, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino, or from a group of the formula:
—X⁵-Q³—
wherein X⁵ is a direct bond or is selected from O, S, SO, SO₂, N(R⁶), CO, CH(OR⁶), CON(R⁶), N(R⁶)CO, SO₂N(R⁶), N(R⁶)SO₂, C(R⁶)₂O, C(R⁶)₂S and C(R⁶)₂N(R⁶), wherein R⁶ is hydrogen or (1-6C)alkyl, and Q³ is (3-7C)cycloalkyl, (3-7C)cycloalkyl-(1-6C)alkyl, (3-7C)cycloalkenyl, (3-7C)cycloalkenyl-(1-6C)alkyl, heterocyclyl or heterocyclyl-(1-6C)alkyl,
and wherein any heterocyclyl group within a substituent on R¹ optionally bears 1, 2 or 3 substituents, which may be the same or different, selected from halogeno, trifluoromethyl, cyano, nitro, hydroxy, amino, carboxy, carbamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino, and N-(1-6C)alkyl-(1-6C)alkanesulfonylamino, or from a group of the formula:
—X⁶—R⁷
wherein X⁶ is a direct bond or is selected from O, N(R⁸) and C(O), wherein R⁸ is hydrogen or (1-6C)alkyl, and R⁷ is halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, (2-6C)alkanoyl-(1-6C)alkyl or (1-6C)alkoxycarbonyl-(1-6C)alkyl,
and wherein any heterocyclyl group within a substituent on R¹ optionally bears 1 or 2 oxo or thioxo substituents;
X¹ is (C(R⁹)₂)ₙ, wherein each R⁹, which may be the same or different, is selected from hydrogen, hydroxy, (1-4C)alkoxy, (1-4C)alkyl, halo(1-4C)alkyl, hydroxy (1-4C)alkyl, (1-4C)alkoxy(1-4C)alkyl, (3-7C)cycloalkyl and (3-7C)cycloalkyl-(1-4C)alkyl, or two groups R⁹ together with the carbon atom(s) to which they are attached form a (3-7C)cycloalkyl ring, and n is 1 or 2, provided that when a group R⁹ is hydroxy or (1-4C)alkoxy, n is 2, and the carbon atom to which the hydroxy or (1-4C)alkoxy group is attached is not also attached to another oxygen or a nitrogen atom;
Q^{a} is a non-aromatic saturated or partially unsaturated heterocyclyl group containing 1 nitrogen heteroatom and optionally 1, 2 or 3 additional heteroatoms selected from O, S and N, and which group is linked to X¹ in Formula I by the nitrogen heteroatom in Q^{a};
q is 0, 1, 2, 3 or 4;
each W, which may be the same or different, is selected from halogeno, trifluoromethyl, cyano, nitro, hydroxy, oxo, amino, carboxy, carbamoyl, sulfamoyl, formyl, mercapto, (1-6C)alkyl, (1-6C)alkoxy, (2-6C)alkenyl, (2-6C)alkynyl, (2-6C)alkenyloxy, (2-6C)alkynyloxy, (1-6C)alkoxycarbonyl, (1-6C)alkylthio, (1-6C)alkylsulfinyl, (1-6C)alkylsulfonyl, N-(1-6C)alkylamino, N,N-di-[(1-6C)alkyl]amino, N-(1-6C)alkylcarbamoyl, N,N-di-[(1-6C)alkyl]carbamoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino, and N-(1-6C)alkyl-(1-6C)alkanesulfonylamino, or from a group of the formula:
—X⁷—R¹⁰
wherein X⁷ is a direct bond or is selected from O, CO and N(R¹¹), wherein R¹¹ is hydrogen or (1-6C)alkyl, and R¹⁰ is selected from (1-6C)alkyl optionally substituted by one or more groups selected from halogeno, hydroxy, (1-6C)alkoxy, cyano, amino, N-(1-6C)alkylamino, N,N-di-[(1-6C)alkyl]amino, (2-6C)alkanoylamino, carbamoyl, N-(1-6C)alkylcarbamoyl, N,N-di-[(1-6C)alkyl]carbamoyl, (2-6C)alkanoyl and (2-6C)alkanoyloxy,
or two W groups form a (1-4C)alkylene bridge, which (1-4C)alkylene bridge optionally bears 1, 2 or 3 substituents, which may be the same or different, selected from halogeno, hydroxy, oxo, (1-6C)alkyl, (1-6C)alkoxy, amino, N-(1-6C)alkylamino and N,N-di-[(1-6C)alkyl]amino;
X² is selected from CH₂C(O), CH₂SO₂, C(O) and SO₂;
Z is selected from hydrogen, (1-6C)alkyl, (2-6C)alkenyl, (2-6C)alkynyl, (3-7C)cycloalkyl, (3-7C)cycloalkyl-(1-4C)alkyl, heterocyclyl, heterocyclyl-(1-4C)alkyl, aryl and aryl-(1-4C)alkyl,
and wherein adjacent carbon atoms in any (2-6C)alkylene chain within a Z substituent are optionally separated by the insertion into the chain of a group selected from O, S, SO, SO₂, N(R¹²) and CO, wherein R¹² is selected from hydrogen and (1-6C)alkyl,
and wherein any CH₂═CH— or HC≡C— group within a Z substituent optionally bears at the terminal CH₂═ or HC≡ position a substituent selected from halogeno, carboxy, carbamoyl,
and wherein any alkyl, alkylene or (3-7C)cycloalkyl group within a Z substituent, optionally bears on one or more halogeno or (1-6C)alkyl substituents or a substituent selected from hydroxy, cyano, amino, carboxy, carbamoyl, sulfamoyl, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino, N-(1-6C)alkyl-(1-6C)alkanesulfonylamino, (3-7C)cycloalkyl and heterocyclyl,
and wherein any aryl or heterocyclyl group within a Z substituent optionally bears one or more substituents selected from halogeno, cyano, nitro, hydroxy, amino, carboxy, carbamoyl, sulfamoyl, trifluoromethyl, (1-4C)alkyl, (2-4C)alkenyl, (2-4C)alkynyl, (1-3C)alkoxy, (1-4C)alkylthio, (1-4C)alkylsulfinyl, (1-4C)alkylsulfonyl, (2-6C)alkanoyl, (1-4C)alkylamino, di-[(1-4C)alkyl]amino, (1-4C)alkoxycarbonyl, N-(1-6C)alkylcarbamoyl and N,N-di-[(1-6C)alkyl]carbamoyl,
and wherein any heterocyclyl group within a Z substituent optionally bears 1 or 2 oxo or thioxo substituents, provided that any of said oxo substituents are not on a carbon atom adjacent to a ring oxygen in the heterocyclyl group;
R²⁰ is hydrogen, (1-6C)alkyl, hydroxy-(2-6C)alkyl or (1-6C)alkoxy(2-6C)alkyl;
a is 1, 2, 3, 4 or 5;
each R³, which may be the same or different, is selected from halogeno, cyano, nitro, hydroxy, amino, carboxy, carbamoyl, sulfamoyl, trifluoromethyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, N-(1-6C)alkylsulfamoyl, and N,N-di-[(1-6C)alkyl]sulfamoyl;
or a pharmaceutically acceptable salt thereof.

2. A quinazoline derivative of the Formula I according to claim 1 of the Formula IA, or a pharmaceutically acceptable salt thereof: 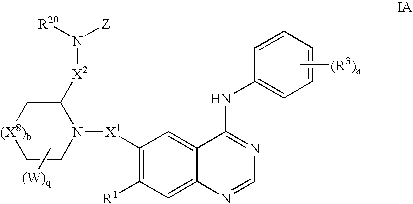
wherein:
R¹ is selected from hydrogen, hydroxy, (1-6C)alkoxy, (2-6C)alkenyloxy, (2-6C)alkynyloxy, or a group of the formula:
Q¹-X³—
wherein X³ is O or S, and Q¹ is (3-7C)cycloalkyl, (3-7C)cycloalkyl-(1-6C)alkyl, (3-7C)cycloalkenyl, (3-7C)cycloalkenyl-(1-6C)alkyl, heterocyclyl or heterocyclyl-(1-6C)alkyl,
and wherein adjacent carbon atoms in any (2-6C)alkylene chain within a R¹ substituent are optionally separated by the insertion into the chain of a group selected from O, S, SO, SO₂, N(R⁴), CO, CH(OR⁴), CON(R⁴), N(R⁴)CO, SO₂N(R⁴), N(R⁴)SO₂, CH═CH and C≡C wherein R⁴ is hydrogen or (1-6C)alkyl,
and wherein any CH₂═CH— or HC≡C— group within a R¹ substituent optionally bears at the terminal CH₂═ or HC≡ position a substituent selected from halogeno, carboxy, carbamoyl, (1-6C)alkoxycarbonyl, N-(1-6C)alkylcarbamoyl, N,N-di-[(1-6C)alkyl]carbamoyl, amino-(1-6C)alkyl, (1-6C)alkylamino-(1-6C)alkyl and di-[(1-6C)alkyl]amino-(1-6C)alkyl or from a group of the formula:
Q²-X⁴—
wherein X⁴ is a direct bond or is selected from CO and N(R⁵)CO, wherein R⁵ is hydrogen or (1-6C)alkyl, and Q² is heterocyclyl or heterocyclyl-(1-6C)alkyl,
and wherein any alkyl or alkylene group within a R¹ substituent optionally bears one or more halogeno, (1-6C)alkyl, hydroxy, cyano, amino, carboxy, carbamoyl, sulfamoyl, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino, or from a group of the formula:
—X⁵-Q³
wherein X⁵ is a direct bond or is selected from O, S, SO, SO₂, N(R⁶), CO, CH(OR⁶), CON(R⁶), N(R⁶)CO, SO₂N(R⁶), N(R⁶)SO₂, C(R⁶)₂O, C(R⁶)₂S and C(R⁶)₂N(R⁶), wherein R⁶ is hydrogen or (1-6C)alkyl, and Q³ is (3-7C)cycloalkyl, (3-7C)cycloalkyl-(1-6C)alkyl, (3-7C)cycloalkenyl, (3-7C)cycloalkenyl-(1-6C)alkyl, heterocyclyl or heterocyclyl-(1-6C)alkyl,
and wherein any heterocyclyl group within a substituent on R¹ optionally bears 1, 2 or 3 substituents, which may be the same or different, selected from halogeno, trifluoromethyl, cyano, nitro, hydroxy, amino, carboxy, carbamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino, and N-(1-6C)alkyl-(1-6C)alkanesulfonylamino, or from a group of the formula:
—X⁶—R⁷
wherein X⁶ is a direct bond or is selected from O, N(R⁸) and C(O), wherein R⁸ is hydrogen or (1-6C)alkyl, and R⁷ is halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, (2-6C)alkanoyl-(1-6C)alkyl or (1-6C)alkoxycarbonyl-(1-6C)alkyl,
and wherein any heterocyclyl group within a substituent on R¹ optionally bears 1 or 2 oxo or thioxo substituents;
X¹ is (C(R⁹)₂)ₙ, wherein each R⁹, which may be the same or different, is selected from hydrogen, hydroxy, (1-4C)alkyl, halo(1-4C)alkyl, hydroxy (1-4C)alkyl, (1-4C)alkoxy(1-4C)alkyl, and n is 1 or 2, or two groups R⁹ together with the carbon atom(s) to which they are attached form a (3-7C)cycloalkyl ring, provided that when a group R⁹ is hydroxy, n is 2, and the carbon atom to which the hydroxy or (1-4C)alkoxy group is attached is not also attached to another oxygen or a nitrogen atom;
each W, which may be the same or different, is selected from halogeno, trifluoromethyl, cyano, nitro, hydroxy, oxo, amino, carboxy, carbamoyl, sulfamoyl, formyl, mercapto, (1-6C)alkyl, (2-6C)alkenyl, (2-6C)alkynyl, (2-6C)alkenyloxy, (2-6C)alkynyloxy, (1-6C)alkoxycarbonyl, (1-6C)alkylthio, (1-6C)alkylsulfinyl, (1-6C)alkylsulfonyl, N-(1-6C)alkylcarbamoyl, N,N-di-[(1-6C)alkyl]carbamoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino, and N-(1-6C)alkyl-(1-6C)alkanesulfonylamino, or from a group of the formula:
—X⁷—R¹⁰
wherein X⁷ is a direct bond or is selected from O, CO and N(R¹¹), wherein R¹¹ is hydrogen or (1-6C)alkyl, and R¹⁰ is (1-6C)alkyl optionally substituted by one or more groups selected from halogeno, hydroxy, (1-6C)alkoxy, cyano, amino, N-(1-6C)alkylamino, N,N-di-[(1-6C)alkyl]amino, (2-6C)alkanoylamino, carbamoyl, N-(1-6C)alkylcarbamoyl, N,N-di-[(1-6C)alkyl]carbamoyl, (2-6C)alkanoyl and (2-6C)alkanoyloxy,
X² is selected from C(O) and SO₂;
Z is selected from hydrogen, (1-6C)alkyl, (2-6C)alkenyl, (2-6C)alkynyl,
and wherein adjacent carbon atoms in any (2-6C)alkylene chain within a Z substituent are optionally separated by the insertion into the chain of a group selected from O, S, SO, SO₂, N(R¹²) and CO, wherein R¹² is selected from hydrogen and (1-6C)alkyl,
and wherein any CH₂═CH— or HC≡C— group within a Z substituent optionally bears at the terminal CH₂═ or HC≡ position a substituent selected from halogeno, carboxy, carbamoyl,
and wherein any alkyl or alkylene group within a Z substituent, optionally bears on one or more halogeno or (1-6C)alkyl substituents or a substituent selected from hydroxy, cyano, amino, carboxy, carbamoyl, sulfamoyl, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino or (3-8)cycloalkyl or heterocyclyl, either of which may be optionally substituted by one or more groups selected from halogeno, cyano, nitro, hydroxy, amino, carboxy, carbamoyl, sulfamoyl, trifluoromethyl, (1-4C)alkyl, (2-4C)alkenyl, (2-4C)alkynyl, (1-3C)alkoxy, (2-4C)alkenyloxy, (2-4C)alkynyloxy, (1-4C)alkylthio, (1-4C)alkylsulfinyl, (1-4C)alkylsulfonyl, (1-4C)alkylamino, di-[(1-4C)alkyl]amino, (1-4C)alkoxycarbonyl;
each R³, which may be the same or different, is selected from halogeno, cyano, nitro, hydroxy, amino, carboxy, carbamoyl, sulfamoyl, trifluoromethyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, N-(1-6C)alkylsulfamoyl, and N,N-di-[(1-6C)alkyl]sulfamoyl
X⁸ is selected from CH₂, O or NR¹³, where R¹³ is hydrogen, halogeno, trifluoromethyl, carboxy, carbamoyl, sulfamoyl, formyl, mercapto, (1-6C)alkyl, (2-6C)alkenyl, (2-6C)alkynyl, (2-6C)alkenyloxy, (2-6C)alkynyloxy, (1-6C)alkoxycarbonyl, (1-6C)alkylthio, (1-6C)alkylsulfinyl, (1-6C)alkylsulfonyl, N-(1-6C)alkylcarbamoyl, N,N-di-[(1-6C)alkyl]carbamoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino, and N-(1-6C)alkyl-(1-6C)alkanesulfonylamino, or from a group of the formula:
—X⁷—R¹⁰
where X⁷ and R¹⁰ are as defined above;
a is 1, 2, 3, 4 or 5;
b is 0 or 1;
q is 0, 1, 2, 3 or 4; and
R²⁰ is hydrogen, (1-6C)alkyl, or (1-6C)alkoxy(2-6C)alkyl.

3. A quinazoline derivative according to claim 1, or a pharmaceutically acceptable salt thereof, wherein Q^{a} is selected from azetidin-1-yl, pyrrolidin-1-yl, piperidino 1,3-thiazolidin-3-yl, morpholino and piperazin-1-yl.

4. A quinazoline derivative according to claim 1 or claim 3, or a pharmaceutically acceptable salt thereof, wherein the group —X²NZR²⁰ is in the ortho (2-) position relative to the ring nitrogen atom in Q^{a} that is attached to X¹.

5. A quinazoline derivative according to claim 2, or a pharmaceutically acceptable salt thereof, wherein b is 0.

6. A quinazoline derivative according to any one of the preceding claims, or a pharmaceutically acceptable salt thereof, wherein R¹ is selected from hydrogen, (1-6C)alkoxy and (1-6C)alkoxy(1-6C)alkoxy.

7. A quinazoline derivative according to any one of the preceding claims, or a pharmaceutically acceptable salt thereof, wherein R¹ is (1-3C)alkoxy.

8. A quinazoline derivative according to any one of the preceding claims, or a pharmaceutically acceptable salt thereof, wherein X¹ is CHR⁹, wherein R⁹ is selected from hydrogen and (1-4C) alkyl.

9. A quinazoline derivative according to any one of the preceding claims, or a pharmaceutically acceptable salt thereof, wherein X¹ is CH₂.

10. A quinazoline derivative according to any one of the preceding claims, or a pharmaceutically acceptable salt thereof, wherein q is 0, 1 or 2 and each W, which may be the same or different, is selected from hydroxy, amino, (1-4C)alkyl, (1-4C)alkoxy, (1-4C)alkylamino, di-[(1-4C)alkyl]amino, hydroxy-(1-4C)alkyl and (1-4C)alkoxy-(1-4C)alkyl.

11. A quinazoline derivative according to any one of the preceding claims, or a pharmaceutically acceptable salt thereof, wherein X² is C(O).

12. A quinazoline derivative according to any one of the preceding claims, or a pharmaceutically acceptable salt thereof, wherein R²⁰ is hydrogen.

13. A quinazoline derivative according to any one of the preceding claims, or a pharmaceutically acceptable salt thereof, wherein Z is selected from hydrogen, (1-3C)alkyl, (2-3C)alkenyl (2-3C)alkynyl, hydroxy-(2-3C)alkyl, (1-3C)alkoxy-(2-3C)alkyl and cyano-(1-3C)alkyl.

14. A quinazoline derivative according to any one of the preceding claims, or a pharmaceutically acceptable salt thereof, wherein Z and R²⁰ are both hydrogen.

15. A quinazoline derivative according to any one of the preceding claims, or a pharmaceutically acceptable salt thereof, wherein the anilino group at the 4-position on the quinazoline ring in Formula I is selected from 3-chloro-4-fluoroanilino, 3-bromo-2-fluoroanilino, 3-chloro-2-fluoroanilino, 2-fluoro-5-chloroanilino, 3-bromoanilino and 3-ethynylanilino.

16. A quinazoline derivative according to any one of the preceding claims, or a pharmaceutically acceptable salt thereof, wherein the anilino group at the 4-position on the quinazoline ring in Formula I is 3-chloro-2-fluoroanilino.

17. A quinazoline derivative of the Formula I according to claim 1 of the Formula IB, or a pharmaceutically acceptable salt thereof: 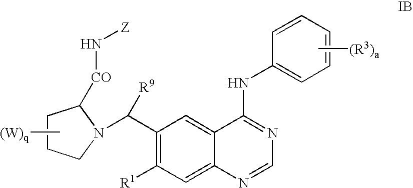
R¹ is (1-4C)alkoxy;
R⁹ is hydrogen or methyl;
q is 0, 1 or 2;
each W, which may be the same or different, is as defined in claim 1;
Z is selected from hydrogen and (1-3C)alkyl;
a is 1 or 2; and
each R³, which may be the same or different is selected from fluoro, chloro, bromo and ethynyl.

18. A quinazoline derivative of the Formula I according to claim 17, or a pharmaceutically acceptable salt thereof wherein the anilino group at the 4-position on the quinazoline ring is selected from 3-chloro-4-fluoroanilino and 3-bromo-2-fluoroanilino.

19. A quinazoline derivative according to claim 1 which is selected from:
1-({4-[(3-chloro-2-fluorophenyl)amino]-7-methoxyquinazolin-6-yl}methyl)-L-prolinamide;
1-({4-[(3-chloro-2-fluorophenyl)amino]-7-methoxyquinazolin-6-yl}methyl)-D-prolinamide;
(4R)-1-({4-[(3-chloro-2-fluorophenyl)amino]-7-methoxyquinazolin-6-yl}methyl)-4-hydroxy-L-prolinamide;
(4S)-1-({4-[(3-chloro-2-fluorophenyl)amino]-7-methoxyquinazolin-6-yl}methyl)-4-hydroxy-L-prolinamide;
(4S)-1-({4-[(3-chloro-2-fluorophenyl)amino]-7-methoxyquinazolin-6-yl}methyl)-4-hydroxy-D-prolinamide;
(4R)-1-({4-[(3-chloro-2-fluorophenyl)amino]-7-methoxyquinazolin-6-yl}methyl)-4-hydroxy-D-prolinamide;
1-({4-[(3-chloro-4-fluorophenyl)amino]-7-methoxyquinazolin-6-yl}methyl)-L-prolinamide;
1-({4-[(3-chloro-4-fluorophenyl)amino]-7-methoxyquinazolin-6-yl}methyl)-D-prolinamide;
(4R)-1-({4-[(3-chloro-4-fluorophenyl)amino]-7-methoxyquinazolin-6-yl}methyl)-4-hydroxy-D-prolinamide;
(4R)-1-({4-[(3-Chloro-2-fluorophenyl)amino]-7-methoxyquinazolin-6-yl}methyl)-4-hydroperoxy-D-prolinamide;
1-({4-[(3-chloro-2-fluorophenyl)amino]-7-methoxyquinazolin-6-yl}methyl)-D-proline; and
1-({4-[(3-chloro-2-fluorophenyl)amino]-7-methoxyquinazolin-6-yl}methyl)-N,N-dimethyl-L-prolinamide;
or a pharmaceutically acceptable sale thereof.

20. A quinazoline derivative according to claim 1 which is selected from:
(4R)-3-({4-[(3-chloro-2-fluorophenyl)amino]-7-methoxyquinazolin-6-yl}methyl)-1,3-thiazolidine-4-carboxamide;
(3S)-1-({4-[(3-chloro-2-fluorophenyl)amino]-7-methoxyquinazolin-6-yl}methyl)-3-hydroxy-L-prolinamide;
(4R)-1-({4-[(3-chloro-2-fluorophenyl)amino]-7-methoxyquinazolin-6-yl}methyl)-4-ethoxy-D-prolinamide;
1-({4-[(3-chloro-2-fluorophenyl)amino]-7-methoxyquinazolin-6-yl}methyl)-2-methylprolinamide; and
(1S,5R)-3-({4-[(3-Chloro-2-fluorophenyl)amino]-7-methoxyquinazolin-6-yl}methyl)-3-azabicyclo[3.1.0]hexane-2-carboxamide
or a pharmaceutically acceptable salt thereof.

21. A pharmaceutical composition which comprises a quinazoline derivative of the Formula I, or a pharmaceutically acceptable salt thereof, according to any one of the preceding claims, in association with a pharmaceutically-acceptable diluent or carrier.

22. A quinazoline derivative of the Formula I, or a pharmaceutically acceptable salt thereof, according to any one of claims 1 to 20, for use as a medicament.

23. Use of a quinazoline derivative of the Formula I, or a pharmaceutically acceptable salt thereof, as defined in any one of claims 1 to 20 in the manufacture of a medicament for use in the production of an anti-proliferative effect in a warm-blooded animal such as a human.

24. Use of a quinazoline derivative of the Formula I, or a pharmaceutically acceptable salt thereof, as defined in any one of claims 1 to 20 in the manufacture of a medicament for use in the treatment of a cancer in a warm-blooded animal such as a human.

25. A method for producing an anti-proliferative effect in a warm-blooded animal, such as a human, in need of such treatment which comprises administering to said animal an effective amount of a quinazoline derivative of the Formula I, or a pharmaceutically acceptable salt thereof, as defined in any one of claims 1 to 20.

26. A method for treating a cancer in a warm-blooded animal, such as a human, in need of such treatment, which comprises administering to said animal an effective amount of a quinazoline derivative of the Formula I, or a pharmaceutically acceptable salt thereof, as defined in any one of claims 1 to 20.

27. A process for the preparation of a quinazoline derivative of the Formula I as defined in claim 1 which comprises:
Process (a):
the reaction of a compound of formula (II): 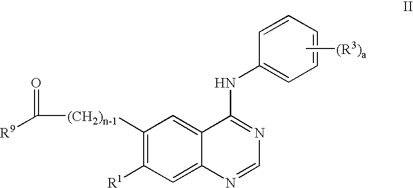
wherein n, a, R¹, R³ and R⁹ are as defined in claim 1, except that any functional group is protected if necessary, with a compound of formula (III): 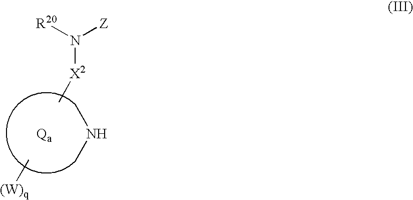
wherein X², W, Z, R²⁰ b and Q^{a} are as defined in claim 1, except that any functional group is protected if necessary; or
Process (b):
the reaction of a compound of formula (XX): 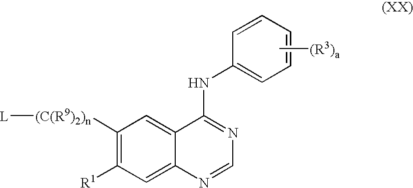
wherein R¹, R³, R⁹, n and a are as defined in claim 1, except that any functional group is protected if necessary, and L is a leaving group, with a compound of formula (III) as defined above in relation to Process (a); or
Process (c)
for the preparation of quinazoline derivatives of the Formula I wherein X² is C(O), the coupling, conveniently in the presence of a suitable base, of a quinazoline of the formula (XXI) or a reactive derivative thereof: 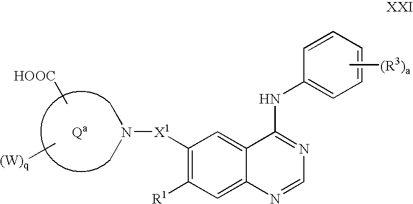
wherein R¹, R³, W, a, q, X¹ and Q^{a} are as defined in claim 1, except that any functional group is protected if necessary, with a compound of the formula XXII, or a salt thereof:
HN(R²⁰)Z XXII
wherein R²⁰ and Z are as defined in claim 1 except that any functional group is protected if necessary; or
Process (d)
the reductive amination of the corresponding quinazoline derivative of the Formula I which contains an NH group with an appropriate aldehyde; or
Process (e)
for the production of those quinazoline derivatives of the Formula I wherein R¹ is hydroxy, the cleavage of a quinazoline derivative of the Formula I wherein R¹ is a (1-6C)alkoxy group; or
Process (f)
for the production of those quinazoline derivatives of the Formula I wherein R¹ is linked to the quinazoline ring by an oxygen atom, by coupling a compound of the formula (XXIII): 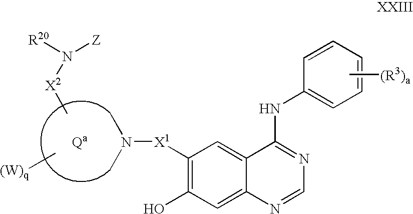
wherein R³, R²⁰, Z, W, a, q, X¹, X² and Q^{a} are as defined in claim 1, except that any functional group is protected if necessary, with a compound of the formula R^{1′} OH wherein R^{1′} is one of the oxygen linked groups as hereinbefore defined for R¹ in claim 1, except that any functional group is protected if necessary;
and thereafter, if necessary (in any order):
(i) converting a quinazoline derivative of the Formula I into another quinazoline derivative of the Formula I;
(ii) removing any protecting group that is present by conventional means; and
(iii) forming a pharmaceutically acceptable salt.
